(19) United States
(12) Patent Application Publication
(10) Pub. No.: US 2024/0041301 A1
(43) Pub. Date: Feb. 8, 2024

(54) BODY CAVITY NAVIGATION DEVICES AND METHODS OF USE

(71) Applicant: Drive Medical, Inc., Claremont, CA (US)

(72) Inventors: Yan Shi ZHAO, Claremont, CA (US); Corbett W. STONE, Buckeye, AZ (US); Matthew DITROLIO, Claremont, CA (US); Jamil Nader ELBANNA, Claremont, CA (US)

(73) Assignee: Drive Medical, Inc., Claremont, CA (US)

(21) Appl. No.: 18/268,967

(22) PCT Filed: Dec. 21, 2021
(86) PCT No.: PCT/US21/64730

§ 371 Date: Jun. 21, 2023

Related U.S. Application Data
(60) Provisional application No. 63/129,454, filed on Dec. 22, 2020.

(51) Int. Cl. A61B 1/00

(52) U.S. Cl. CPC A61B 1/00082; A61B 1/0016; A61B 2017/00818

(57) ABSTRACT

The present disclosure relates to a device configured to move within a body cavity, such as the gastrointestinal tract, in particular, the small intestine, and methods of using the device. The presently disclosed device may be self-driving, e.g., through the use of one or more traction-motion element, and the articulation of a tip of the device may be controlled and fine tuned. The presently disclosed device may be used in a variety of body cavities such as a vascular body lumen, a digestive body lumen, a respiratory body lumen, or a urinary body lumen, for example, for endoscopic purposes, for delivering a substance into the body cavity, for removing a substance or tissue from the body cavity, for capturing an image of the body cavity, and/or for performing an operation of a tissue or organ using the device.

### CROSS-REFERENCE TO RELATED APPLICATION

This application claims priority from U.S. provisional application No. 63/129,454 filed Dec. 22, 2020, entitled “Devices and Systems for Body Cavities and Methods of Use,” which is incorporated herein by reference in its entirety for all purposes.

### FIELD

The present disclosure relates to a device configured to move within a body cavity, such as the gastrointestinal tract, in particular, the small intestine, and methods of using the device for endoscopic purposes, for delivering a substance into the body cavity, for removing a substance or tissue from the body cavity, for capturing an image of the body cavity, and/or for performing an operation of a tissue or organ using the device. The presently disclosed device may be self-driving, and the articulation of a tip of the device may be controlled and fine tuned. The presently disclosed device may be used in a variety of body cavities such as a vascular body lumen, a digestive body lumen, a respiratory body lumen, or a urinary body lumen.

### BACKGROUND

The current endoscopic procedures, such as esophagogastroduodenoscopy (EGD), colonoscopy, enteroscopy, etc., involve intensive human operation of the systems. For instance, it is generally known that a gastrointestinal examination uses an endoscope having a flexible insertion section. In inserting the above-mentioned endoscope into deep part of the digestive tract, e.g., the small intestine, when the insertion section is inserted thereinto while being pushed, a force is hardly transmitted to the distal end of the insertion section because the intestine is complicatedly curved. It is, therefore, difficult to insert the insertion section into deep part. Oftentimes, even when it is possible to insert an endoscope into deep part, it takes a long time, causes discomfort and pain, and requires sedation. There is need for a device that is easy to use and causes less discomfort. The present disclosure addresses these and other needs.

### SUMMARY

In some aspects, provided herein is a device configured to move within a body cavity, the device comprising: a) a support (e.g., an elongated support, such as a tubular structure such as a tether); b) a proximal radially expandable element and a distal radially expandable element positioned along the length of the support, optionally wherein the radially expandable elements are independently controllably expandable, wherein the radially expandable elements comprise outer surfaces configured to frictionally engage a wall of a body cavity, and wherein the distal radially expandable element is fixed relative to the support; and c) a locomotion system comprising: i) a proximal locomotion element having a part that is fixed relative to the proximal radially expandable element and slidable along the length of the support, such that the proximal radially expandable element is slidable along the length of the support, and ii) a distal locomotion element having a part fixed relative to the support, wherein the locomotion system effects sliding movement of the proximal radially expandable element along the length of the support, thereby effecting relative movement between the radially expandable elements and movement of the device within the body cavity. In any of the embodiments herein, the distal locomotion element can comprise a part fixed relative to the distal radially expandable element, e.g., the distal locomotion element or part thereof can be directly or indirectly fixed to the distal radially expandable element or a part thereof.

In some aspects, provided herein is a device configured to move within a body cavity, comprising: a support (e.g., a tubular structure such as a tether); controllably expandable elements positioned along the length of the support, from proximal to distal: a proximal locomotion element (e.g., a first longitudinally expandable element), a proximal radially expandable element (e.g., a first radially expandable element), a distal locomotion element (e.g., a second longitudinally expandable element), a distal radially expandable element (e.g., a second radially expandable element), wherein the radially expandable elements comprise outer surfaces configured to frictionally engage a wall of a body cavity, wherein the first radially expandable element or the second radially expandable element is slidable along the support relative to the support, and wherein the proximal and the distal locomotion elements are configured to effect relative movement between the radially expandable elements (e.g., relative movement between the outer surfaces configured to frictionally engage the body cavity wall), thereby effecting movement of the device within the body cavity. In any of the embodiments herein, the proximal end of the proximal locomotion element (e.g., the first longitudinally expandable element) and the distal end of the distal locomotion element (e.g., the second longitudinally expandable element) may have a fixed distance between each other. In any of the embodiments herein, the positions of the proximal end of the proximal locomotion element (e.g., the first longitudinally expandable element) and the distal end of the distal locomotion element (e.g., the second longitudinally expandable element) may be fixed relative to each other. In any of the embodiments herein, the positions of the proximal end of the proximal locomotion element (e.g., the first longitudinally expandable element) and the distal end of the distal locomotion element (e.g., the second longitudinally expandable element) may be fixed relative to the support.

In any of the embodiments herein, the proximal locomotion element may be moveable relative to the distal locomotion element. In any of the embodiments herein, the proximal locomotion element may be moveable relative to the support. In any of the embodiments herein, the distal locomotion element may be fixed relative to the support. In any of the embodiments herein, the proximal locomotion element may be moveable relative to the support, whereas the distal locomotion element may be fixed relative to the support, or vice versa. In any of the embodiments herein, the proximal locomotion element may comprise a floating element that is moveable relative to the support (e.g., capable of being pulled by a cable and/or pushed by a rod along the length of the support), whereas the distal locomotion element may comprise a wheel that is fixed relative to the support. In any of the embodiments herein, a cable may be fixed to the floating element and engage the wheel, such that the cable may be guided by the wheel (e.g., the cable may engage a groove such as a V-groove in the wheel) and pulled in the proximal direction, thereby pulling the floating element (and the proximal radially expandable element attached thereto) in the distal direction.

In any of the embodiments herein, the proximal end of the proximal locomotion element may be moveable relative to the distal end of the distal locomotion element. In any of the embodiments herein, the proximal end of the proximal locomotion element may be moveable relative to the support. In any of the embodiments herein, the distal end of the distal locomotion element may be fixed relative to the support. In any of the embodiments herein, the proximal end of the proximal locomotion element may be moveable relative to the support, whereas the distal end of the distal locomotion element may be fixed relative to the support.

In any of the embodiments herein, the distal locomotion element may comprise a part fixed directly or indirectly to the distal expandable element, for instance, a distal end of the distal locomotion element may be directly or indirectly to a proximal end of the distal expandable element. In any of the embodiments herein, the distal locomotion element may comprise a part fixed directly or indirectly to the promixal expandable element, for instance, a proximal end of the distal locomotion element may be directly or indirectly to a distal end of the proximal expandable element.

In any of the embodiments herein, the proximal locomotion element may comprise a part fixed directly or indirectly to the proximal expandable element, for instance, a distal end of the proximal locomotion element may be directly or indirectly to a proximal end of the proximal expandable element.

In any of the embodiments herein, the support may be an elongated support comprising a tubular wall and a lumen, optionally wherein the lumen is a central lumen.

In any of the embodiments herein, one or both of the expandable elements and/or one or both of the locomotion elements can be in fluid or gas communication with one or more chambers, one or more channels, one or more tubes, and/or one or more wires in the central lumen. In any of the embodiments herein, any one or more of the expandable elements and locomotion elements can be independently controlled.

In any of the embodiments herein, the locomotion elements may be configured to expand or collapse along the length of the elongated support, optionally wherein the locomotion elements are configured to expand or collapse only along the length of the elongated support and/or are not radially expandable.

In any of the embodiments herein, the proximal and the distal radially expandable elements may be capable of expanding radially outwardly to engage a wall of a body cavity, optionally wherein friction augmenting features are molded into the proximal and/or distal radially expanding elements.

In any of the embodiments herein, alternating extensions and retractions of a distance between the outer surfaces of the proximal and the distal radially expandable elements may effect movement of the device within the body cavity.

In any of the embodiments herein, the device may further comprise an articulation element capable of effecting articulation of a distal end of the device, optionally wherein the distal end of the device is the distal end of the elongated support or the distal end of the device directly or indirectly engages the distal end of the elongated support. In any of the embodiments herein, the articulation element may enable steering of the device, optionally wherein the device comprises a machine vision element that digitally recognizes structures assisting in navigation and identifying anomalies such as lesions and polyps, optionally wherein the machine vision assists in navigation and/or transmitting location of structures such as when moving from the large intestine to the small intestine. In any of the embodiments herein, the articulation element may comprise one or more motors and/or one or more cables. In any of the embodiments herein, the articulation element may comprise one or more closed loop cables configured to effect articulation, e.g., by pulling a distal end of the device.

In any of the embodiments herein, the device may further comprise one or more channels not in connection with the expandable elements, e.g., fluid and/or gas connection with an inside space of the expandable element(s).

In any of the embodiments herein, the proximal radially expandable element can comprise or be a proximal balloon. In any of the embodiments herein, the distal radially expandable element can comprise or be a distal balloon. In any of the embodiments herein, the proximal radially expandable element may directly or indirectly engage one or more floating elements configured to slide along the length of the elongated support, thereby sliding the proximal radially expandable element along the length of the elongated support.

In any of the embodiments herein, the locomotion system may comprise two longitudinally expandable elements. In any of the embodiments herein, the locomotion system may comprise a proximal longitudinally expandable element and a distal longitudinally expandable element, optionally wherein the longitudinally expandable elements are independently controllably expandable, and optionally wherein the longitudinally expandable elements each comprises a structure independently selected from the group consisting of a compliant balloon, a semi-compliant balloon, a pressure balloon, and a bellow.

In any of the embodiments herein, the locomotion system can comprise a pulley system. In any of the embodiments herein, the pulley system may comprise a proximal floating element, a distal wheel, and a cable connected to the proximal floating element and engaging the distal wheel.

In some aspects, provided herein is a device configured to move within a body cavity, the device comprising: a) an elongated support; b) a proximal radially expandable element and a distal radially expandable element positioned along the length of the elongated support, wherein the radially expandable elements comprise outer surfaces configured to frictionally engage a wall of a body cavity, and wherein the distal radially expandable element is fixed relative to the elongated support; c) a locomotion system comprising a proximal longitudinally expandable element and a distal longitudinally expandable element connected by a floating seal, wherein: i) the proximal longitudinally expandable element is proximal to the proximal radially expandable element, and the floating seal is fixed relative to the proximal radially expandable element and slidable along the length of the elongated support, such that the proximal radially expandable element is slidable along the length of the elongated support, and ii) the distal longitudinally expandable element is proximal to the distal radially expandable element, and the distal end of the distal longitudinally expandable element is fixed relative to the elongated support, wherein the locomotion system effects sliding movement of the proximal radially expandable element along the length of the elongated support, thereby effecting relative movement between the radially expandable elements and movement of the device within the body cavity.

In any of the embodiments herein, the proximal and the distal longitudinally expandable elements can be configured to expand or collapse along the length of the elongated support, optionally wherein the proximal and the distal longitudinally expandable elements configured to expand or collapse only along the length of the elongated support and/or are not radially expandable. In any of the embodiments herein, alternating expansion and collapsing of the proximal and the distal longitudinally expandable elements may change the distance between the proximal end of the proximal longitudinally expandable element and the distal end of the distal longitudinally expandable element.

Alternatively, in any of the embodiments herein, alternating expansion and collapsing of the proximal and the distal longitudinally expandable elements do not need to change the distance between the proximal end of the proximal longitudinally expandable element and the distal end of the distal longitudinally expandable element. In any of the embodiments herein, alternating expansion and collapsing of the proximal and the distal longitudinally expandable elements do not need to change the distance between the distal end of the proximal longitudinally expandable element and the proximal end of the distal longitudinally expandable element. For instance, the distal end of the proximal longitudinally expandable element and the proximal end of the distal longitudinally expandable element can be separated by a floating seal (e.g., attached to a proximal radially expandable element) and the dimension of the floating seal along the length of the support is fixed.

In any of the embodiments herein, the distance between the proximal end of the proximal longitudinally expandable element and the distal end of the distal longitudinally expandable element can be pre-determined. In any of the embodiments herein, the distance can be more than about 1 cm, more than about 2 cm, more than about 3 cm, more than about 4 cm, more than about 5 cm, more than about 6 cm, more than about 7 cm, more than about 8 cm, more than about 9 cm, or more than about 10 cm. In any of the embodiments herein, the distance can be from about 3 cm to about 6 cm, from about 6 cm to about 9 cm, or from about 9 cm to about 12 cm.

In any of the embodiments herein, the maximum distance between the radially expandable elements along the length of the support during the movement of the device (e.g., driven by the alternating expansion and collapsing of the proximal and the distal longitudinally expandable elements) within a body cavity can be pre-determined. In any of the embodiments herein, the maximum distance can be more than about 1 cm, more than about 2 cm, more than about 3 cm, more than about 4 cm, more than about 5 cm, more than about 6 cm, more than about 7 cm, more than about 8 cm, more than about 9 cm, or more than about 10 cm. In any of the embodiments herein, the maximum distance can be from about 3 cm to about 6 cm, from about 6 cm to about 9 cm, or from about 9 cm to about 12 cm.

In any of the embodiments herein, the distance and/or the maximum distance between the radially expandable elements along the length of the support during the movement of the device may be adjusted according to the curvature of the body cavity.

In any of the embodiments herein, the expansion of the proximal and/or the distal longitudinally expandable elements can be effected by positive pressure, optionally wherein negative pressure is proactively and alternatively applied to the longitudinally expandable elements in order to evacuate previously applied positive pressure, and optionally wherein the proximal and/or the distal longitudinally expandable elements do not passively deflate. In any of the embodiments herein, the expansion of the proximal longitudinally expandable element and the collapsing of the distal longitudinally expandable element can effect sliding movement of the proximal radially expandable element along the length of the elongated support. In any of the preceding embodiments, the collapsing of the proximal longitudinally expandable element and the expansion of the distal longitudinally expandable element can effect movement of the distal radially expandable element, e.g., when the distal radially expandable element is not expanded to engage a wall of the body cavity.

In some aspect, provided herein is a method for locomotion of the device of any of the embodiments herein through a body cavity, the method comprising: i. expanding the proximal radially expandable element radially outwardly to engage a wall of the body cavity, thereby fixing the proximal radially expandable element to a first position in the body cavity; ii. expanding (e.g., using positive pressure) the distal longitudinally expandable element along the elongated support to increase the distance between the proximal and the distal radially expandable elements; iii. expanding the distal radially expandable element radially outwardly to engage a wall of the body cavity; iv. retracting the proximal radially expandable element radially inwardly; v. retracting (e.g., using negative pressure) the distal longitudinally expandable element, and/or expanding (e.g., using positive pressure) the proximal longitudinally expandable element along the elongated support, thus effecting sliding movement of the proximal radially expandable element along the elongated support and decreasing the distance between the proximal and the distal radially expandable elements; and vi. optionally expanding the proximal radially expandable element radially outwardly to engage a wall of the body cavity, thereby fixing the proximal radially expandable element to a second position in the body cavity, optionally the second position is distal to the first position. In any of the embodiments herein, the method can further comprise step vii. expanding (e.g., using positive pressure) the distal longitudinally expandable element along the elongated support to increase the distance between the proximal and the distal radially expandable elements. In any of the embodiments herein, the method can further comprise repeating steps i-vi.

In some aspects, provided herein is a method for locomotion of the device of any of the embodiments herein through a body cavity, the method comprising: i. expanding the distal radially expandable element radially outwardly to engage a wall of the body cavity, thereby fixing the distal radially expandable element to a first position in the body cavity; ii. expanding (e.g., using positive pressure) the proximal longitudinally expandable element along the elongated support, thus effecting sliding movement of the proximal radially expandable element along the elongated support and decreasing the distance between the proximal and the distal radially expandable elements; iii. expanding the proximal radially expandable element radially outwardly to engage a wall of the body cavity; iv. retracting the distal radially expandable element radially inwardly; v. retracting (e.g., using negative pressure) the proximal longitudinally expandable element, and/or expanding (e.g., using positive pressure) the distal longitudinally expandable element along the elongated support; and vi. optionally expanding the distal radially expandable element radially outwardly to engage a wall of the body cavity, thereby fixing the distal radially expandable element to a second position in the body cavity, optionally the second position is distal to the first position. In any of the embodiments herein, the method can further comprise step vii. expanding (e.g., using positive pressure) the proximal longitudinally expandable element along the elongated support to decrease the distance between the proximal and the distal radially expandable elements. In any of the embodiments herein, the method can further comprise repeating steps i-vi.

In some aspects, provided herein is a device configured to move within a body cavity, the device comprising: a) an elongated support; b) a proximal radially expandable element and a distal radially expandable element positioned along the length of the elongated support, wherein the radially expandable elements comprise outer surfaces configured to frictionally engage a wall of a body cavity, and wherein the distal radially expandable element is fixed relative to the elongated support; c) a pulley system comprising: i) a proximal floating element that is fixed relative to the proximal radially expandable element and slidable along the length of the elongated support, such that the proximal radially expandable element is slidable along the length of the elongated support, ii) a distal wheel fixed relative to the elongated support, and iii) a cable connected to the proximal floating element and engaging the distal wheel, such that the cable is configured to pull the proximal floating element in the distal or proximal direction, wherein the pulley system effects sliding movement of the proximal radially expandable element along the length of the elongated support, thereby effecting relative movement between the radially expandable elements and movement of the device within the body cavity. In any of the embodiments herein, the cable can comprise or be a closed loop cable.

In any of the embodiments herein, movement of the cable can effect movement of the proximal radially expandable element along the elongated support, thereby effecting alternative extensions and retractions of a distance between the outer surfaces of the proximal and the distal radially expandable elements along the length of the elongated support. In any of the embodiments herein, the radially expandable elements are independently controllably expandably, and, optionally wherein the elongated support further comprises one or more medium channels separately connected to the radially expandable elements, optionally wherein the medium comprises gas, liquid or a mixture thereof and optionally wherein the medium comprises a vapor, and/or wherein the elongated support further comprises one or more wires or channels for a camera or sensor, optionally wherein the sensor is a pressure sensor and/or an image sensor, and/or wherein the elongated support houses or engages an endoscope assembly.

In any of the preceding embodiments, pulling the proximal floating element in the proximal direction while the proximal radially expandable element is collapsed and while the distal radially expandable element is expanded to engage a body cavity results in the proximal radially expandable element moving proximally within the body cavity, thereby increasing the distance between the proximal and the distal radially expandable elements. In any of the embodiments herein, pulling the proximal floating element in the proximal direction while the proximal radially expandable element is expanded to engage a body cavity and while the distal radially expandable element is collapsed may result in the distal radially expandable element moving distally within the body cavity, thereby increasing the distance between the proximal and the distal radially expandable elements. In any of the embodiments herein, pulling the proximal floating element in the distal direction while the proximal radially expandable element is collapsed and while the distal radially expandable element is expanded to engage a body cavity may result in the proximal radially expandable element moving distally within the body cavity, thereby decreasing the distance between the proximal and the distal radially expandable elements. In any of the embodiments herein, pulling the proximal floating element in the distal direction while the proximal radially expandable element is expanded to engage a body cavity and while the distal radially expandable element is collapsed may result in the distal radially expandable element moving proximally within the body cavity, thereby decreasing the distance between the proximal and the distal radially expandable elements.

In some aspects, provided herein is a method for locomotion of the device of any of the embodiments herein, through a body cavity, the method comprising: i. expanding the proximal radially expandable element radially outwardly to engage a wall of the body cavity, thereby fixing the proximal radially expandable element to a first position in the body cavity; ii. pulling the proximal floating element in the proximal direction along the elongated support while the distal radially expandable element is collapsed, thereby increasing the distance between the proximal and the distal radially expandable elements; iii. expanding the distal radially expandable element radially outwardly to engage a wall of the body cavity; iv. retracting the proximal radially expandable element radially inwardly; v. pulling the proximal floating element in the distal direction, thus effecting sliding movement of the proximal radially expandable element along the elongated support and decreasing the distance between the proximal and the distal radially expandable elements; and vi. expanding the proximal radially expandable element radially outwardly to engage a wall of the body cavity, thereby fixing the proximal radially expandable element to a second position in the body cavity, optionally wherein the second position is distal to the first position. In any of the embodiments herein, the method can further comprise step vii. pulling the proximal floating element in the proximal direction along the elongated support while the distal radially expandable element is collapsed, thereby increasing the distance between the proximal and the distal radially expandable elements. In any of the embodiments herein, the method can further comprise repeating steps i-vi.

In some aspects, provided herein is a method for locomotion of the device of any of the embodiments herein through a body cavity, the method comprising: i. expanding the distal radially expandable element radially outwardly to engage a wall of the body cavity, thereby fixing the distal radially expandable element to a first position in the body cavity; ii. pulling the proximal floating element in the distal direction along the elongated support while the proximal radially expandable element is collapsed, thereby decreasing the distance between the proximal and the distal radially expandable elements; iii. expanding the proximal radially expandable element radially outwardly to engage a wall of the body cavity; iv. retracting the distal radially expandable element radially inwardly; v. pulling the proximal floating element in the proximal direction, thus effecting sliding movement of the distal radially expandable element forward along the elongated support and increasing the distance between the proximal and the distal radially expandable elements; and vi. expanding the distal radially expandable element radially outwardly to engage a wall of the body cavity, thereby fixing the distal radially expandable element to a second position in the body cavity, optionally wherein the second position is distal to the first position. In any of the embodiments herein, the method can further comprise step vii. pulling the proximal floating element in the distal direction along the elongated support while the proximal radially expandable element is collapsed, thereby decreasing the distance between the proximal and the distal radially expandable elements. In any of the embodiments herein, the method can further comprise repeating steps i-vi.

In any of the embodiments herein, the device provided herein can further comprise an articulation element capable of effecting articulation of a distal end of the device, optionally wherein the distal end of the device is the distal end of the elongated support or the distal end of the device directly or indirectly engages the distal end of the elongated support. In any of the embodiments herein, the articulation element can enable camera visualization and/or steering of the device (e.g., one comprising an endoscope assembly), optionally wherein the device comprises machine vision elements that digitally recognize structures assisting in navigation and identifying anomalies such as lesions and polyps, optionally wherein the machine vision assists in navigation and/or transmitting location of structures such as when moving from the large to small intestine. In any of the embodiments herein, the articulation element can comprise one o more motors and/or one or more cables. In any of the embodiments herein, the articulation element may comprises one or more closed loop cables configured to effect articulation.

In any of the embodiments herein, the method provided herein can further comprise capturing an image of the body cavity through a channel of the device. In any of the embodiments herein, the method can further comprise delivering a substance into the body cavity through a channel of the device. In any of the embodiments herein, the method can further comprise removing a substance into the body cavity through a channel of the device. In any of the embodiments herein, the method can further comprise performing an operation on a tissue within the body cavity through a channel of the device.

In any of the embodiments herein, the body cavity can be a vascular body lumen, a digestive body lumen, a respiratory body lumen, or a urinary body lumen. In any of the embodiments herein, the digestive body lumen can be a gastrointestinal tract. In any of the embodiments herein, the gastrointestinal tract can comprise small intestine. In any of the embodiments herein, the gastrointestinal tract can comprise duodenum, jejunum, and/or ileum. In any of the embodiments herein, the gastrointestinal tract can comprise large intestine. In any of the embodiments herein, the gastrointestinal tract can comprise colon. In any of the embodiments herein, a device disclosed herein can move in the gastrointestinal tract, e.g., from one part of large intestine to another part of large intestine, from large intestine to small intestine, and/or from one part of small intestine to another part of small intestine. In any of the embodiments herein, the gastrointestinal tract can comprise esophagus. In any of the embodiments herein, the gastrointestinal tract can comprise stomach.

In any of the embodiments herein, the expandable elements can be connected to the elongated support (e.g., tubular structure such as tether) using an elastic O-ring that mechanically holds the expandable elements; using adhesive only securing the edges of the expandable elements; mechanically securing the edges of an expandable element by a deformable material such as a metal by swaging or radially compressing it around the expandable element; or by a combination thereof.

In any of the embodiments herein, the device does not require a locking mechanism for directly or indirectly interlocking the first and second radially expandable elements to prevent relative movement between these radially expandable elements. In any of the embodiments herein, a method of using the device for moving inside a body cavity does not require a step of interlocking the first and second radially expandable elements to prevent relative movement between the radially expandable elements. In some embodiments, provided herein are devices and methods comprising one or more mechanisms and/or steps of effectuating and/or controlling relative movement between the radially expandable elements, in addition to mechanisms and/or steps of articulating a tip of the device.

### BRIEF DESCRIPTION OF THE DRAWINGS

FIGS. 1A-1D show the structure and application of an exemplary device comprising two controllably expandable elements, an outer tube, an inner tube, a connector, and an actuator.

FIG. 2A shows an exemplary device comprising an inner tube, an outer tube, two controllably expandable elements (e.g., balloons), an actuating mechanism (e.g., a stepper motor), and an articulation mechanism. FIGS. 2B-2C show an exemplary device comprising a screw/nut connector.

FIG. 3 shows an exemplary process of using a device disclosed herein, including the placement and movement of the device inside a body cavity such as a gastrointestinal tract.

FIGS. 4A-4D show various exemplary configurations of the medium channels controlling the inflation and/or deflation of the balloons.

FIG. 5 shows exemplary mechanisms for the rotation and tilting of the tip portion of the inner tube, in order to guide the inner tube to move in various directions, e.g., following the curves of the body cavity.

FIG. 6 shows exemplary mechanisms for a chamber in the base of the tip portion of the inner tube to effect articulation of the distal end portion of the inner tube.

FIG. 7 shows exemplary mechanisms involving an additional chamber on top of the base to effect articulation of the distal end portion of the inner tube.

FIG. 8 shows an exemplary chamber within the base.

FIG. 9 shows an exemplary air channel going through the inner tube body and connecting to the base.

FIG. 10 shows an exemplary mechanism involving the rotation of a servo motor placed proximal to the first stepper motor for the screw/nut, in order for the rotation be to transmitted to the base to effect articulation.

FIG. 11 shows an exemplary water/air/suction channel traversing the inner tube.

FIG. 12 shows an exemplary optical fiber or wire of a camera traversing the inner tube.

FIG. 13 shows an exemplary camera channel and an exemplary water/air/suction channel passing the round base through an air-sealed tunnel.

FIG. 14 shows an exemplary guidewire attached to the outer tube and the inner tube.

FIG. 15A shows an exemplary actuating mechanism comprising a controllably expandable telescoping structure to effect longitudinal movement of the device.

FIG. 15B shows an exemplary shape memory alloy actuating mechanism to effect longitudinal movement of the device.

FIG. 15C shows an exemplary mechanism to effect longitudinal movement of the device.

FIG. 16 shows exemplary controllably expandable structures configured to expand or contract longitudinally, thereby effecting longitudinal movement of the device.

FIG. 17A-17D show exemplary controllably expandable structures configured to expand or contract, thereby effecting longitudinal movement of the device and/or articulation of the device, e.g., articulation of the distal portion of the inner tube in a direction transverse to the longitudinal axis of the body portion of the inner tube.

FIG. 18 shows four exemplary pressure balloons configured to expand or contract, thereby effecting longitudinal movement of the device and/or articulation of the device, e.g., articulation of the distal portion of the inner tube in a direction transverse to the longitudinal axis of the body portion of the inner tube.

FIG. 19 shows an exemplary helical mechanism to effect longitudinal movement of the device and/or articulation of the device, e.g., articulation of the distal portion of the inner tube in a direction transverse to the longitudinal axis of the body portion of the inner tube.

FIG. 20A-20F show exemplary bellows designs, which may be used to effect longitudinal movement of the device and/or articulation of the device, e.g., articulation of the distal portion of the inner tube in a direction transverse to the longitudinal axis of the body portion of the inner tube.

FIG. 21 and FIG. 22 show exemplary bellows designs comprising one or more supporting structures.

FIGS. 23A-23H show exemplary quarter bellows designs.

FIG. 24 shows an exemplary device comprising a propulsion mechanism (e.g., hydraulic propulsion) and an articulation mechanism.

FIGS. 25A-25C show exemplary devices comprising a hydraulic articulation and/or propulsion mechanism.

FIG. 26A shows an exemplary device comprising a cable articulation and/or propulsion mechanism. FIG. 26B shows an exemplary device comprising a motor/pulley articulation mechanism. FIG. 26C shows an exemplary device comprising a linear servo motor propulsion mechanism.

FIGS. 27-28 show an exemplary device disclosed herein configured to move within a body cavity and comprising one or more traction-motion element.

FIG. 29 shows crosssections of an exemplary device as a controllably expandable element of the device expands and contracts.

FIG. 30 shows the crosssection of an exemplary device comprising one or more aperture connecting an controllably expandable element and the central lumen of a support (e.g., tubular structure such as tether).

FIG. 31 shows the crosssection of an exemplary device comprising a slit connecting an controllably expandable element and the central lumen of a support (e.g., tubular structure such as tether).

FIG. 32 shows an example where the controllably expandable elements of a device work in a controlled and coordinated fashion to provide both traction and motor functions, driving the device within the body cavity.

FIG. 33 shows an exemplary device comprising a plurality of controllably expandable elements (e.g., traction-motion balloons) disposed on the support (e.g., tubular structure such as tether), where the controlled and coordinated expansion and/or contraction (e.g., sequential inflation) of the controllably expandable elements provide both traction and motor functions, driving the device within the body cavity.

FIG. 34 shows an exemplary step-wise method for locomotion of a device relative to a tube. In panel a, the proximal/first radially expandable element expands radially outwardly to engage a wall of the tube and fixing itself to the tube, while the distal/second radially expandable element is not radially outwardly expanded. In panel b, the distal/second longitudinally expandable element (propulsion balloon on the left) is expanded while the proximal/first radially expandable element is expanded, thereby pushing the distal end of the device forward. In panel c, the distal/second radially expandable element is expanded radially outwardly to engage a wall of the tube. In panel d, the proximal/first radially expandable element is retracted (e.g., deflated). In panel e, the distal/second longitudinally expandable element is retracted while the proximal/first longitudinally expandable element (propulsion balloon on the right) is expanded, thereby bringing the device forward. Steps in panels a and b are repeated in a′ and b′ to propel the distal end of the device further forward.

FIG. 35 shows an exemplary device comprising a proximal propulsion balloon, a proximal traction balloon, a distal propulsion balloon, and a distal traction balloon. The proximal end of the proximal propulsion balloon and the distal end of the distal propulsion balloon can be fixed relative to the tether, while the proximal traction balloon can be floating. The distal traction balloon can be fixed relative to the tether.

FIG. 36 shows an exemplary device where a floating proximal traction balloon comprises a dynamic separator seal (e.g., a floating seal).

FIG. 37 shows an exemplary device comprising a distal tip that can articulate, e.g., while the device is in a stationary mode, for steering and/or camera positioning.

FIG. 38 shows an exemplary device comprising a proximal propulsion balloon, a proximal traction balloon, a distal propulsion balloon, and a distal traction balloon. The proximal end of the proximal propulsion balloon and the distal end of the distal propulsion balloon can be fixed relative to the tether, while the proximal traction balloon can be floating. The distal traction balloon can be fixed relative to the tether.

FIG. 39 shows an exemplary device where a floating proximal traction balloon comprises a dynamic separator seal (e.g., a floating seal) and a distal tip that can articulate. The articulation of the tip can be controlled by articulation cable(s).

FIGS. 40A-40C show an articulation of the tip which is controlled by articulation cable.

FIG. 41 shows an exemplary step-wise method for locomotion of a device relative to a tube. Panel a shows the overall design of the device, wherein the proximal radially expandable element (labeled “proximal traction body”) is connected to the cable in a pulley system, thus dividing the pulley system into a proximal part and a distal part (proximal and distal propulsion bodies). In panel b, the proximal part of the pulley system (propulsion body) is expanded by movement of the cable while the distal radially expandable element (traction body) is expanded, thereby pushing the proximal radially expandable element (traction body) forward. In panel c, the proximal radially expandable element is pushed forward, and expanded radially outwardly to engage a wall of the tube. Panels d and e show a method for reverse locomotion, wherein the proximal radially expandable element is retracted (e.g., deflated), the distal radially expandable element is expanded, and the distal part of the pulley system (propulsion body) is expanded, thereby bringing the device backward. Steps in panels d and e may be repeated while the proximal radially expandable element is expanded and the distal radially expandable element is retracted, thereby propel the distal end of the device further forward.

FIG. 42 shows an exemplary device propelling a proximal radially expandable element forward in a distal direction in a curved tract.

FIG. 43A shows a portion of a split tube along which the radially expandable elements (traction bodies) in FIGS. 40 and 41 may slide, wherein the split tube comprises a plurality of longitudinal slits.

FIG. 43B shows a follower (43C) with cable attachment parts (43D) that may be installed on the cable of the exemplary device shown in FIGS. 40 and 41 to facilitate movement of the cable and locomotion of the device. FIG. 43C shows the cross section of the follower. For example, the follower may be attached to the proximal radially expandable element (traction body) as shown in FIG. 43D, wherein a longitudinal section of the follower is shown.

FIGS. 44A-44B and FIGS. 45A-45B show an exemplary device comprising a flexible region between two radially expandable structures, where the flexible region may comprise one or more compression springs and/or one or more cables.

FIG. 46 shows an exemplary method of using the device.

FIGS. 47A-47B and FIGS. 48A-48C show an exemplary device comprising a flexible region between two radially expandable structures, where the flexible region may comprise a plurality of sets of cables.

FIG. 49 shows an exemplary method of using the device.

### DETAILED DESCRIPTION

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. All publications, including patent documents, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

The small intestine is the longest and most important section of the intestine, where 90% of the digestion and absorption of nutrients and minerals occurs. Despite providing such vital functions for the human body, the small intestine remains difficult in accessibility, a ‘black box’ for physicians due to its length and location. The current understanding of small bowel physiology is limited, making diagnosis and treatment of small bowel diseases challenging to physicians. This has significant repercussions for patients. One common example is the management of Crohn's disease (CD), a disease primarily affecting the small bowel. In recent years, the prevalence of CD increased about 31% with a significant increase in Crohn's morbidity such as associated neoplastic transformation, social, psychological, financial repercussions, and impaired patient health-related quality of life (HRQoL). However, as the dearth of effective endoscopic tools makes successful performance of the procedure difficult and prohibitively expensive, diagnosis of CD is often hard to achieve, as direct mucosal examination and tissue sampling are required to make a definitive diagnosis but extremely difficult to perform. Irritable bowel syndrome (IBS) is another example in which lack of effective endoscopic tools harms patients. IBS is the most commonly diagnosed GI condition, accounting for approximately 30 percent of all referrals to gastroenterologists. IBS is associated with increased health care costs and is the second highest cause of work absenteeism. Though GI dysmotility is traditionally believed to be one of the etiologies for IBS, knowledge on small bowel motility remains nearly nil due to the lack of effective endoscopic tools for exploration into this research area.

Prevention of small bowel cancer associated with Peutz-Jeghers syndrome is a significant unmet health need. Peutz-Jeghers syndrome (PJS) is an autosomal dominant syndrome characterized by multiple hamartomatous polyps in the gastrointestinal tract, mucocutaneous pigmentation, and an increased risk of gastrointestinal and non-gastrointestinal cancer. Peutz-Jeghers syndrome (PJS) is rare with an estimated prevalence of 1:8000 to 1:200,000 births. Males and females are equally affected. Gastrointestinal hamartomatous polyps are present in most patients with PJS, and the polyps most commonly occur in the small bowel (60 to 90 percent). Gastrointestinal polyps develop in the first decade of life and most patients become symptomatic between the ages of 10 and 30. The distribution of gastrointestinal cancers in PJS is similar to that of the hamartomatous polyps, and carcinoma arising in hamartomas has been clearly documented. The average age of developing a malignancy in PJS is 42 years, and the lifetime cancer risk in the small bowel is 13%. Due to the increased risk for GI cancer in individuals with PJS, baseline endoscopic screening of the gastrointestinal tract includes upper gastrointestinal endoscopy (esophagogastroduodenoscopy), video capsule endoscopy (VCE), and colonoscopy, beginning at age eight years. The subsequent screening interval is based on the findings at baseline examination. Magnetic resonance enterography (MRE) is an alternative imaging modality for individuals with PJS for whom capsule endoscopy cannot be performed. Endoscopic polypectomy should be performed for small bowel polyps>1 cm in size in order to reduce the risk of polyp-related complications such as malignancy. Currently, a multitude of technologies need to be employed to manage small bowel polyps in PJS (e.g., capsule endoscopy and MRE) to identify and localize small bowel polyps/cancer, followed by deep enteroscopy to remove polyps. Peutz-Jeghers syndrome (PJS) and associated small bowel polyps and cancers represent a clear unmet need. The ability to diagnose and treat PJS patients to prevent cancer would be a boon, particularly in the small bowel, where few effective tools currently exist to diagnose and remove polyps.

The inherent problems in understanding the small bowel and its associated diseases indicate that exploration of small intestinal pathophysiology is crucial to advancing the GI field in evaluation and treating small bowel diseases, and improving patient care outcomes. There is a need for cost-effective endoscopic tools, and the present disclosure addresses this and other needs.

In some aspects, provided herein is an endoscopic tool that is 1) of a miniature size to minimize patient's discomfort in order to eliminate sedation; 2) capable of traveling the long intestinal tract; and/or 3) capable of providing diagnosis and therapy if needed. In some aspects, provided herein is an endoscopic tool that meets all of the three criteria above.

In some aspects, provided herein is a device, including use of a double balloon mechanism, an extended tether, and advances in camera technology. For example, double balloon enteroscopes have shown the effectiveness of the double balloon mechanism, and current colonoscopes have tethers, and high-resolution cameras. In addition, advances in soft robotics enable a compact and flexible motor system that is suitable to navigate through a tortuous path such as the GI tract. The present invention uses these previous innovations as a springboard to build a new and better tool to conduct gastrointestinal polypectomies for PJS patients.

In some aspects, the present disclosure provides advantages over existing technologies. For example, wireless capsule endoscopy (WCE) avoids sedation but is limited by its inability to intervene, quality of visualization, and random passage through the intestines; deep enteroscopy (DE) is capable of endoscopic interventions but is limited by lengthy, often incomplete procedures, general anesthesia requirements, special physician training, and significant risks, costs, and time associated with the procedures. In some aspects, the present disclosure provides devices and methods that address one or more of these disadvantages.

Much research has also been conducted on robotic endoscopic capsules to address these problems, but appears to have reached a bottle neck. The three ‘ground challenges’ in designing such a robot are 1) active locomotion, and 2) enabling diagnostic and 3) therapeutic functionalities due to limitations on size and power supply. External and internal locomotion are being developed to achieve active locomotion. Magnetic field mechanisms for external locomotion have been researched. Significant drawbacks are cost and difficulty in obtaining effective visualization and locomotion. Internal locomotion has significant advantages over magnetic fields, but the excessive internal encumbrance needed to suit the size of a miniature robot (e.g., the presence of motors/actuators, transmission mechanisms, and high-capacity power modules) limits its success. Other challenges to effective locomotion are intrinsic to the intestine: slippery surface and accordion effect from intestinal deformation when the robot advances. Diagnostic and therapeutic functionalities are unable to be addressed at the same time. Again, development is constrained by size and power supply.

In some aspects, provided herein are devices and methods that address all three challenges. In some aspects, provided herein are devices and methods based on at least a double balloon mechanism (DBM), utilized in double balloon enteroscopy (DBE), and soft robotics. DBE utilizes two alternating balloons to propel the scope. Its successful clinical application has confirmed its safety feature on tissue and intestine surface holding validity. These provide strong evidence for its feasibility to overcome the slippery environment. To overcome the accordion effect, generally a long linear stroke is desired; however, a long and rigid stroke will produce discomfort. Design of soft robotics will allow us to provide a flexible motion. In some embodiments herein, a 3D printed bellow and/or manually assembled multiple longitudinally aligned balloons are used to achieve motor function, respectively. In some embodiments, the device is pneumatically powered externally, and the freedom of external power supply eliminates the large payload in current robotic capsules. Because of the extra space saved, there is room to carry accessories needed for diagnostic and therapeutic functionalities.

In some embodiments, provided herein is a flexible self-driven endoscopic robotic capsule with interventional capacity. In some embodiments, the device is small enough to avoid requiring anesthesia. In some embodiments, the device is a disposable device that reduces or eliminates infection risks associated with cleaning of re-usable scopes in current practice.

In some embodiments, using the device disclosed herein makes it possible to perform controlled examination and intervention in the entire small bowel without sedation. In some embodiments, the space for accessories in the device allows physicians to continue using existing accessory tools, which allows smooth transition from traditional endoscopy to a device disclosed herein, while avoiding the cost associated with training new users or developing specific accessories. In some embodiments, the device comprises a bellow motor design that provides both articulation and actuation motor functions and maintains small motor size at the same time, which is essential to the development for both surgical device and soft robotics.

In some embodiments, the device disclosed herein may be used to examine the colon, optionally combining evaluation of small bowel and colon into a one-step test. In some embodiments, the device disclosed herein reduces eliminates needs for anesthesia and eliminates risks of infection associated with cleaning of re-usable scopes. In some embodiments, the device disclosed herein enables delivery of other diagnostic means such as motility catheter, one or more sensor (such as ultrasound sensors), tissue sampling for research purpose. In some embodiments, the device disclosed herein enables delivery drugs to the target area more easily (without sedation) and more precisely.

In some embodiments, the device disclosed herein may be used to conduct examination of and perform intervention in the small bowel at the same time. It is small enough to avoid requiring anesthesia and is a disposable device that eliminates the possibility of cross-infection. In some embodiments, the device disclosed herein provides effective locomotion all while enabling diagnostic and therapeutic functionalities. In some embodiments, the device disclosed herein comprises a bellow motor and/or a balloon motor. In some embodiments, the device disclosed herein comprises a bellow motor is a de novo motor that provides both articulation and actuation motor functions while maintaining a small motor size at the same time.

In some embodiments, the device disclosed herein reduces or eliminates needs for anesthesia, reduces or eliminates risks of infection associated with cleaning of re-usable scopes, and enables delivery of other diagnostic means such as motility catheters, tissue sampling for research purpose, one or more sensor (such as ultrasound sensors), and delivery drugs to the target area more easily (without sedation) and more precisely.

In some embodiments, the device disclosed herein comprises double balloons such as the traction balloons disclosed herein. In some embodiments, the device disclosed herein comprises a soft robotic member comprising a 3D printed bellow and/or multiple longitudinally aligned balloons to achieve motor function.

In some embodiments, the device disclosed herein comprises a soft robotic motor comprising bellows, e.g., a bellow motor, and the bellow motor is configured to elongate more than about 1 cm, more than about 2 cm, more than about 3 cm, more than about 4 cm, more than about 5 cm, more than about 6 cm, more than about 7 cm, more than about 8 cm, more than about 9 cm, or more than about 10 cm. In some embodiments, the bellow motor is configured to elongate from about 3 cm to about 6 cm, from about 6 cm to about 9 cm, or from about 9 cm to about 12 cm. In some aspects, the longer elongation increases effectiveness and reduces procedure time.

In some embodiments, the device disclosed herein comprises a soft robotic motor comprising bellows, e.g., a bellow motor. In some embodiments, the bellow motor is configured to elongate from 3 cm to 6 cm, and can be powered by externally supplied air, which will eliminates the large payload in the current robotic capsules. In some embodiments, the bellow motor comprises a central open space, e.g., to carry accessories that include cameras, power supply, working channel, etc. In some embodiments, the bellow motor is entirely 3D printed and satisfies the requirements for flexibility, strength, and elasticity. In some embodiments, the bellow is approximately 5 cm at rest and able to expand approximately 3 cm.

In some embodiments, the device disclosed herein comprises a soft robotic motor comprising multiple balloons such as balloons enclosed in an expandable sheath, e.g., a balloon motor, and the balloon motor is configured to elongate more than about 1 cm, more than about 2 cm, more than about 3 cm, more than about 4 cm, more than about 5 cm, more than about 6 cm, more than about 7 cm, more than about 8 cm, more than about 9 cm, or more than about 10 cm. In some embodiments, the bellow motor is configured to elongate from about 3 cm to about 6 cm, from about 6 cm to about 9 cm, or from about 9 cm to about 12 cm. In some aspects, the longer elongation increases effectiveness and reduces procedure time.

In some embodiments, the device disclosed herein comprises a soft robotic motor comprising multiple balloons such as balloons enclosed in an expandable sheath, e.g., a balloon motor. In some embodiments, the balloon motor is configured to elongate from 3 cm to 6 cm, and can be powered by externally supplied medium (e.g., air, a gas, or a liquid), which will eliminates the large payload in the current robotic capsules. In some embodiments, the balloon motor comprises a central open space, e.g., to carry accessories that include cameras, power supply, working channel, etc. In some embodiments, the balloon motor combines four longitudinally aligned catheter balloons housed in a custom-made expendable sheath. In some embodiments, the balloon motor is approximately 2.5 cm at rest, expandable up to 6 cm.

In some embodiments, the device disclosed herein comprises a linear stroke of at least 3 cm, for example, to overcome the accordion effect from intestinal deformation during robotic advancements.

Provided herein is a device configured to move within a body cavity. In some embodiments, the device comprises a double-balloon system comprising an actuating or driving mechanism, as well as an articulation mechanism to navigate the complicated curves of a body cavity such as the GI tract. The device may be used but is not made exclusively for enteroscopy. It can be used in any part of the gastrointestinal tract. For example, the device may be used as a colonoscope for technically difficult cases. The device may be used for endoscopic retrograde cholangiopancreatography, e.g., in patients with Roux-en-Y anastomosis in which an endoscopic approach to the papilla of Vater is impossible with regular endoscopic insertion. In some embodiments, the present device provides not only improved accessibility to a distal portion of the GI tract, e.g., the small intestine.

In some embodiments, the present device provides not only improved accessibility to the deep small intestine, but also the ability to control the device tip in any part of the intestine. Precise control of the device tip is possible at any point in the intestine because the movement of the device is controlled from the gripped point by the balloon on the inner tube and/or the balloon on the outer tube, which can be set at any point.

In some embodiments, the present device may be used in place of or in conjunction with a traditional capsule endoscopy and/or balloon-based. Capsule endoscopy is suitable for the initial work-up of nonobstructive small intestinal disorders because it is discomfort-free and does not require the patient to be confined to a medical facility. Abnormal findings detected by a capsule can be confirmed by the presently disclosed device with biopsy examination, and endoscopic treatment can be performed using the device disclosed herein. In particular, small intestinal strictures, which are a contraindication for capsule endoscopy, can be explored by the device disclosed herein. In some embodiments, the device disclosed herein may be used to perform endoscopic balloon dilation. Moreover, in cases of capsule retention at a stricture, the capsule can be retrieved by the device disclosed herein and the stricture can be dilated endoscopically using the device.

In some embodiments, provided herein is a gastrointestinal (GI) navigation and delivery device. In some aspects, the device is designed to navigate through the gastrointestinal system with no or much less human manipulation during the navigation, as compared to conventional endoscopy. In some embodiments, the device is a self-driving device. In some embodiments, the device minimizes or ends the need for sedation. In some embodiments, the device also cuts the procedural cost that is associated with supporting staff, medical supplies, medications, and hospital stay.

In some embodiments, provided herein is a gastrointestinal navigation and delivery device capable of delivering medication to one or more target region within a body cavity. In some embodiments, provided herein is a device configured to deliver an endoscope, a diagnostic capsule, a diagnostic catheter such as a manometry catheter, a therapeutic device such as a stent, a tube, and other device or composition to one or more desired region within a body cavity.

In some embodiments, provided herein is a device configured to drive a capsule endoscopy for the small bowel and colon through the GI tract with a controlled speed and direction. In some embodiments, the device disclosed herein is configured to carry the task for bowel preparation, which is a very unpleasant process and the huge obstacle for people to adhere to colon cancer screening recommendation.

In one aspect, provided herein is a lumen navigation and delivery device comprising a first body section with a proximal end and a distal end, a second body section with a proximal end and a distal end, and a tip section with a proximal end and a distal end wherein the proximal end of the tip section is attached to the distal end of the second body section and the first and second body sections are attached to and capable of sliding within one another in a telescopic fashion. In some embodiments, the first body section, the second body section, and the tip section each comprise an elongated support (e.g., tubular structure such as tether).

In some embodiments, the first body section is larger in size than the second body section such that the second body section is capable of sliding within the first body section. In other embodiments, the second body section is larger in size than the first body section such that the first body section is capable of sliding within the second body section.

In certain embodiments, an inflatable balloon is fixed to the outer wall of the proximal end of the outer tube. In some embodiments, one or more annular inflatable balloons are fixed to the outer wall of the proximal end of the outer tube. In particular embodiments, two spherical inflatable balloons are fixed opposite one another to the outer wall of the proximal end of the outer tube. In certain embodiments, a plurality of spherical inflatable balloons are attached, fixed in position relative to one another, to the outer wall of the proximal end of the outer tube and are arranged substantially evenly in a circular pattern to form an annular-like configuration around the outer tube.

In certain embodiments, an inflatable balloon is fixed to the outer wall of the distal end of the tip section of the inner tube. In some embodiments, one or more annular inflatable balloons are fixed to the outer wall of the distal end of the tip section of the inner tube. In particular embodiments, two spherical inflatable balloons are fixed opposite one another to the outer wall of the distal end of the tip section of the inner tube. In certain embodiments, a plurality of spherical inflatable balloons are attached, fixed in position relative to one another, to the outer wall of the distal end of the tip section and are arranged substantially evenly in a circular pattern to form an annular-like configuration around the tip section.

In certain embodiments, the inflation and deflation of the balloons are controlled by the injection of fluid. In some embodiments, fluid to each balloon is delivered via one or more channels fixed along the outer and inner tubes. In particular embodiments, the one or more channels delivering fluid to the balloons attached to the first tube are fixed to the outer wall of the first tube. In some embodiments, the one or more channels delivering fluid to the balloons attached to the first tube are fixed to the inner wall of the first tube. In some embodiments, the one or more channels delivering fluid to the balloons attached to the inner tube is fixed to the outer wall of the inner tube. In some embodiments, the one or more channels delivering fluid to the balloons attached to the inner tube is fixed to the inner wall of the inner tube.

In certain embodiments, the balloons are made of a material with memory of desired shapes. In some embodiments, the balloons will have a pre-set maximum pressure. In particular embodiments, the balloons incorporates certain adhesive properties. In certain embodiments, the balloons incorporate microfibrillar adhesives from polydimethylsiloxane.

In some embodiments, the gastrointestinal navigation and delivery device disclosed herein comprises an inner tube and an outer tube. In some embodiments, the inner tube moves forward to reach its distance, and may be anchored on the bowel wall by inflating the balloon at the distal end of the inner tube. Then, the outer tube follows by moving forward over the inner tube. Once the outer tube is in place, it is anchored on the bowel wall by inflating the balloon at the proximal end of the outer tube. At this time, the balloon on the inner tube is deflated and moves forward. Once the inner tube reaches its distance, the balloon on the inner tube advances to a more distal position within the body cavity such as the GI tract. Then, the inner tube is anchored onto the bowel wall by inflating its associated balloon, and the outer tube deflates its associated balloon to move forward over the inner tube. The process continues until it reaches a destination, such as a more distal destination in the GI tract, e.g., the small intestine. In any of the embodiments disclosed herein, the tubes can be made of vinyl or polyurethane materials.

In any of the embodiments disclosed herein, the balloons can be made of a material that has memory of the desired shape. In any of the embodiments disclosed herein, the balloons can incorporate certain adhesive properties such as microfibrillar adhesives (e.g., from polydimethylsiloxane (PDMS)), UV-activated adhesives, epoxy or cyanoacrylates to generate traction. In any of the embodiments disclosed herein, the balloons may have treads molded onto the surface to increase friction. In any of the embodiments disclosed herein, an over molding process can be used to increase the thickness of the balloons and add treads to the surface of the balloons. In any of the embodiments disclosed herein, the balloons can be made of custom molded polyurethane, latex, polyisoprene or Pbax. In any of the embodiments disclosed herein, the balloons can be made of polyisoprene for its biocompatibility and desired rigidness. In any of the embodiments disclosed herein, the balloons can be circumferentially wrapping around the inner and/or outer tubes. In any of the embodiments disclosed herein, the device can comprise multiple balloons at the same longitudinal position.

In any of the embodiments disclosed herein, the balloons can have a pre-set maximum pressure (thus maximum inflation) and memory to prevent trauma to bowel wall or cause bowel perforation. For example, in some cases, the maximum allowable pressure directly applied to the bowel wall is 1.55-4.37 PSI. In some embodiments, the pressure limit is measured during use by measuring the bowel wall pressure using a sensing array. In some embodiments, the sensing array comprises MEMS pressure sensors placed inside the traction balloons.

In any of the embodiments disclosed herein, the inflation and/or deflation of the balloons may be controlled, for example, by injecting and/or drawing a gas (such as air) or a fluid via thin tubing along the inner and outer tubes, respectively. In some embodiments, there is provided a tube or an air channel that is along the outside of the outer or inner tube or inside the outer or inner tube, for each balloon, respectively. In some embodiments, there is provided a tube or an air channel that is along the outside of the outer or inner tube for each balloon, respectively. In some embodiments, there is provided a tube or an air channel that is inside the outer or inner tube, for each balloon, respectively. In some embodiments, a part of the tube or air channel is along the outside of the outer or inner tube, while another part of the tube or air channel is inside the outer or inner tube, for each balloon, respectively.

In some embodiments, the device comprises a structure similar to screw and nut, for the inner and outer tubes to move relative to each other. In some embodiments, the screw is inside the inner tube but connected to the outer tube via a stepper motor. An exemplary stepper motor is the commercially available SM3.4-20 from Minebea or vendors. In some embodiments, the stepper motor connects to the outer tube via two arms. In some embodiments, the inner tube connects to a nut which is fixed onto the inner tube. In some embodiments, the nut moves along the screw. In some embodiments, the rotation of the screw enables the nut and the inner tube to move along the outer tube. In some embodiments, with the nut and/or screw moving in one direction and the outer tube being kept stationary by its balloon, the inner tube moves forward; with the nut and/or screw moving in the other direction, and when the inner tube is kept stationary by its balloon, the outer balloon moves forward. Using the same mechanism, both tubes can also move backwards. In some embodiments, the stepper motor connects to a proximal end of the screw to provide the movements. In some embodiments, the stepper motor are connected to the proximal portion of the outer tube via two arms that are fixed onto the outer tube.

In some embodiments, a plurality of longitudinal slits are located on the walls of the inner tube. For example, two longitudinal slits may be provided on the opposite walls of the inner tube. In some embodiments, two arms extend from stepper motor for the screw through the slits and are fixed onto the outer tube. In some embodiments, the plurality of longitudinal slits provide space for the inner tube and outer tube to slide forward and backward, while slidably connecting the inner tube and outer tube during the movements, e.g., in order to prevent the two tubes from disengaging each other (e.g., the distal portion of the inner tube may slide completely into the outer tube or the inner tube may slide completely outside the outer tube) and/or control the maximum/minimum distance between the two balloons, during alternating extensions and retractions of the distance between the two balloons.

In some embodiments, the moving mechanism is advantageous over the current endoscopy in that the device drives itself forward instead of an operator pushing it forward from outside of the body a long distance away. In some embodiments, the mechanism avoids the stretching of the bowel, bowel wall and mesentery, thereby decreasing pain and consequently requiring less sedation and operation time.

In some embodiments, the distal end of the inner tube has an opening for camera. In some embodiments, the device comprises a camera at least part of which is in the inner tube. In some embodiments, the device comprises a light source, e.g., a light source for the camera. In some embodiments, the distal end of the inner tube has an opening for air and/or water. In some embodiments, the distal end of the inner tube has an opening for an irrigation and/or suction channel. In some embodiments, the inner tube can be tapered down in diameter if needed toward the distal end, especially when only an opening for camera and an opening or an irrigation and/or suction channel are needed. In some embodiments, the camera can be a fiber optic camera such as a miniature CMOS image sensor (e.g., NanEye by AMS AG), a camera used in capsule endoscopy, or a wireless camera that is often used in mini drones. In some embodiments, the very distal end of the inner tube is oval or round in shape to minimize trauma to the bowel wall.

In some embodiments, the inner tube comprises two portions, a distal tip portion and a proximal body portion. In some embodiments, the proximal end section of the inner tube tip is connected to the body of inner tube via a motor. In some embodiments, the motor is at a proximal end of the tip portion (and/or at a distal end of the body portion) and connected to a round base that can be inflated and/or deflated to form an asymmetrical shape. In some embodiments, the asymmetrically inflated base enables the tilting of the tip. In some embodiments, the base can rotate in 360 degree fashion that is controlled by another motor, for example, a servo motor or a stepper motor. In some embodiments, by combining the base rotation and tilting the tip, the tip portion of the inner tube is capable of guiding the inner tube to move in various directions. This feature is advantageous for navigating the GI tract, particularly the small intestine.

In some embodiments, the round base is a flexible conduit-like structure, except it is asymmetrical and has a hinge at one side. The hinge can be an actual hinge, such as a mechanical hinge with two parts that pivot relative to each other. In some embodiments, the hinge can be an extension from the distal section of the inner tube that is made of a material that is strong enough and yet can be bent repeatedly.

In some embodiments, the round base is a chamber that comprises a relatively rigid material (e.g., plastic) on both top and bottom surfaces and an elastic material with shape memories on the side. In some embodiments, the top surface (distal) of the round base is the base of the inner tube base. In some embodiments, the bottom surface (proximal) of the round base is separated from the distal surface of the inner tube body and connected with a motor, such as the servo or stepper motor which is connected with the body portion of the inner tube.

In some embodiments, the space between the round base and the distal surface of the inner tube body is small enough to allow the free rotation of the round base. In some embodiments, the chamber of the round base can maintain an angle from 0 degree to 180 degree at the hinge by inflating the base chamber. In some embodiments, if more than 90 degree at the hinge is needed, another chamber that is on top of the first one can be provided to share the same hinge with the first chamber. In some embodiments, in order to maintain an angle between 90 degree and 180 degree at the hinge, another chamber can be provided on top of the first one. In some embodiments, when the chamber returns to its original position with 0 degree at the hinge, there is still some room maintained between the top surface and the lower surface of the chamber. In some embodiments, the distance between the two surfaces depends on the thickness of the folded flexible conduit. In some embodiments, when the angle is at or at about 0 degree, the intra-chamber pressure can be maintained close to zero or even slightly negative to keep the tip of the inner tube and body of the inner tube as one unit.

In some embodiments, the inflation is achieved by a gas (such as sterilized air), a liquid or fluid, or a mixture thereof (such as vapor). In some embodiments, inside the round base, there is a thin cuboid shaped chamber that can be inflated asymmetrically to a triangular shape, thereby inflating the round base to a desired angle. In some embodiments, the cuboid shaped chamber extends across a diameter of the round base but leaves space for one or more flexible tube, e.g., for the air/water/suction channel and the camera cable to pass through the round base. In some embodiments, there is an air channel going through the inner tube body and connect to the round base via a flexible conduit. In some embodiments, regulation of the inflation and rotation of the round base is achieved by a computer program that receives feedback from device, such as from the camera or a sensor, such as a pressure sensor at the tip of the inner tube. Therefore, in coordination with the camera or sensor at the tip of the inner tube, the inner tube recognizes the direction of the bowel lumen and guides the direction of the tube movements.

In some embodiments, the motor on the round base is a servo motor that has a sufficiently small size. In some embodiments, a stepper motor is used, or the servo motor can be placed proximal to the first stepper motor for the screw/nut and connected to the round base with a stiff thin wire that can accurately transmit servo motor's rotation to a pin on the round base via one or more gear.

In any of the preceding embodiments, the device further comprises a controller system. In some embodiments, the controller system comprises one or more pumps, such as the NEMA 17 stepper motor driven pumps. In some embodiments, the controller system comprises one or more valves. In some embodiments, the controller system further comprises a user interface, a computer, and a custom software.

In some embodiments, the water/air/suction channel is a channel traversing the whole inner tube from the proximal inner tube, round base to the distal inner tube. In some embodiments, there is a flexible tube that is fixed to the proximal end of the channel at the distal (tip) section of the inner tube and end freely in the air channel of the inner tube body but fits tightly in the air channel of the inner tube body to maintain a seal. In some embodiments, the flexible tube traverses the round base down to the inner tube body at a length that is long enough to still remain in the tube body's air channel when the round base is inflated to its largest angle at the hinge and when the round base rotates up to 180 degree to both directions (clockwise and counter-clockwise). In some embodiments, the flexible tube is made from a flexible material but does not collapse during operation or is capable to withstand a threshold pressure. In some embodiments, the air channel remains open when the round base is collapsed. In some embodiments, a fiber optic camera such as NanEye is used, the optical fiber can traverse the entire inner tube and/or traverse the round base in the closed relationship to the pin of the servo or stepper motor on the side of the hinge. In some embodiments, this configuration ensures the length of the cable that moves when the round base is rotating is minimum. In some embodiments, the camera cable is secured at the proximal end of the distal inner tube for the same reason as the tube inside the air channel. In some embodiments, a wireless camera is used, and the length of the camera cable that moves when the round base is rotating is not a concern. In some aspects, the air/water irrigation/suction channel and the fiber optic camera traverse the round base through an air-sealed tunnel, for example, to ensure that the round base is air sealed.

In some embodiments, the inner tube body and the outer tube are relatively larger in diameter, while the rest of the inner tube distally has a smaller diameter, for example, carrying only the air/water/suction channel and/or wires (e.g., electric wires for the camera and/or one or more motor). In some embodiments, the electric wires connect the camera and/or motor to a control mechanism outside of the body of a subject.

In some embodiments, the device further comprises a guidewire attached to the outer tube distally and to the inner tube proximally, for example, as a carrier system that allows other mechanisms, such as sample collection, imaging collection, data analysis, delivery of one or more scope and/or catheters etc., to feed over the guidewire and be delivered to a desired location.

In any of the preceding embodiments, the device described herein is configured to move and/or navigate inside a body cavity, such as for intra-vascular or intra-luminal use in other organ systems, e.g., in the respiratory system or the urinary tract.

In some embodiments, provided herein is a controllably expandable structure for use in the device described herein. In some embodiments, the controllably expandable structure comprises a flexible elastomeric hollow double walled part that has a hub at the center and a traction surface at the outside radial surface like a tire. Like a tire the part is inflated with a gas, air, or fluid coming from the center hub. The outside radial surface expands with increasing radius as the hollow part is filled with pressure. It is intended to touch and stick to a lumen (such as the bowel) that is like a tube and have frictional contact with it. Then as the pressure inside the part increases the hub is moved axially due to force applied by the section between the hub and the outside radial surface. The hub is then supported axially by a drive balloon assembly. While the hub is axially supported by the drive balloon assembly, pressure is decreased within the traction-motion balloon and the overall diameter decreases back to the original uninflated shape. This process is repeated in sequence and the assembly is advanced through the body cavity such as a lumen.

In some embodiments, the controllably expandable structure (e.g., an inflatable element such as a balloon-type element) is configured to expand from a collapsed configuration to an expanded configuration, wherein, when in the collapsed configuration, the controllably expandable structure includes one or more fold or ridge extending substantially transverse to a longitudinal axis thereof so that, when a medium (e.g., a gas, a liquid, or a mixture thereof such as a vapor) is supplied thereto, e.g., for inflation, the controllably expandable structure expands substantially along the longitudinal axis. In some embodiments, the controllably expandable structure is connected to an actuator (e.g., an actuator for surgical or endoscopic applications), e.g., via a medium conduit or channel (e.g., an inflation gas/fluid/vapor conduit) or via mechanical structures (such as rods or gears).

In some embodiments, the actuator forms an integral part of the device and remains inside a patient's body during operation of the device. Exemplary actuators include miniaturized motors coupled to the controllably expandable structure, e.g., via the medium conduit or channel and/or mechanical structures.

In some embodiments, the actuator remains outside a patient's body, and the medium conduit or channel extends from the actuator to a proximal end of the controllably expandable structure, thereby coupling the actuator and the controllably expandable structure. In some embodiments, when the actuator is in a first operative configuration, a medium such as an inflation gas, fluid, or vapor is supplied to the controllably expandable structure via the medium conduit or channel. In some embodiments, when the actuator is in a second operative configuration, a medium such as an inflation gas, fluid, or vapor is withdrawn from the controllably expandable structure via the medium conduit or channel. In some embodiments, when the actuator is in a third operative configuration, a certain amount of a medium such as an inflation gas, fluid, or vapor is maintained in the controllably expandable structure, thereby maintaining the state and/or degree of expansion of the controllably expandable structure. In some embodiment, there is no net change of the amount of the medium inside the controllably expandable structure when its degree of expansion is maintained.

In any of the preceding embodiments, the controllably expandable structure may comprise a compliant balloon, a non-compliant balloon, and/or a semi-compliant balloon. The term “compliance” as it relates to balloons describes the degree to which the size of a balloon changes as a function of pressure. Compliant balloons exhibit substantially uniform expansion in response to increasing levels of pressure. A compliant balloon may be “axially compliant” and have a length that exhibits uniform axial expansion during inflation of the balloon; “radially compliant” and have a radius that exhibits uniform radial expansion during inflation of the balloon; or both. Compliant balloons are made of materials that are highly elastic and expand substantially elastically when pressurized. These materials may also have substantial elastic recoil such that upon deflation, compliant balloons return substantially to their original pre-inflation size. Compliant balloon materials include thermosetting and thermoplastic polymers that exhibit substantial stretching upon the application of tensile force. These materials include, but are not limited to, elastomeric materials such as elastomeric varieties of latex, silicone, polyurethane, and polyolefin elastomers. See for example U.S. Pat. No. 7,892,469, which is incorporated herein by reference in its entirety and for all purposes. Compliant balloon materials may be cross-linked or uncross-linked.

Non-compliant balloons, on the other hand, exhibit little expansion in response to increasing levels of pressure. A non-compliant balloon may be “axially non-compliant” and have a length that exhibits little or no axial growth during inflation of the balloon; “radially non-compliant” and have a radius that exhibits little or no radial growth during inflation of the balloon; or both. In the case of a radially non-compliant balloon, the walls of the balloon when uninflated may collapse into folded pleats, allowing the balloon to adopt an axially compressed state. Upon inflation, these pleats unfold, and the axial length of the balloon grows as the radius of the balloon remains substantially unchanged. Non-compliant balloon materials include, but are not limited to, nylon, polyethyleneterephthalate (PET), or various types of polyurethane block copolymers. See Lim et al. Non-compliant balloons can be used to open or expand a body lumen, and due to their predetermined size, they are less likely than compliant balloons either to burst or to rupture or damage lumen walls when highly pressurized. See for example U.S. Pat. No. 8,469,926, which is incorporated herein by reference in its entirety and for all purposes.

In some embodiments, semi-compliant balloons exhibit moderate expansion in response to increasing levels of pressure. In some embodiments, in response to increasing inflation pressure, a semi-compliant balloon expands less than a compliant balloon, but more than a non-compliant balloon. A non-compliant balloon may be “axially semi-compliant,” “radially semi-compliant,” or both. Thus, in some embodiments, with the same pressure, different parts of a semi-compliant balloon may exhibit different degrees of expansion. In other words, a semi-compliant balloon may be designed to expand in more than one direction, but with different degrees of expansion in different directions.

As with non-compliant balloons, semi-compliant balloons may be made of materials that include, but are not limited to, nylon, polyethyleneterephthalate (PET), or polyurethane block copolymers. Semi-compliant balloons maintain in part at least some of the advantages of non-compliant balloons detailed above, but also preserve at least some of the elasticity and flexibility of compliant balloons.

Depending upon the nature of the operation, it may be desirable to further adjust the positioning of an end portion of the inner member and/or an end portion of the outer member. In some embodiments, it is desirable to orient a distal end portion of the inner member at an axis transverse to the longitudinal axis of a body portion of the device, such as a body portion of the inner tube. The transverse movement of the end portion relative to the body portion of the device may be referred to as “articulation.” In some embodiments, articulation is accomplished by a pivot (or articulation) joint being placed between the end portion and the body portion. This articulated positioning permits an operator of the presently disclosed device to more easily engage tissue in some instances and/or navigate the device through a complicatedly curved body cavity, such as the GI tract. In combination of the self-driving mechanisms disclosed herein, the device may be used to gain access to deep parts that are complicatedly curved, such as the small intestine. In some embodiments, articulated positioning advantageously allows the end portion of the device to be positioned in the body cavity without being blocked by tissue inside the body cavity.

In some embodiments provided herein, the device comprises a hydraulic actuator in between the proximal/first controllably expandable element and the distal/second controllably expandable element of the device, and engagement of the hydraulic actuator effects the sliding movement between the outer member and the inner member of the device. In other embodiments, mechanical actuators like lead screws or cable assemblies can be used instead. In some embodiments, the device further comprises a plurality of soft, compliant fluid channels running longitudinally through the device, and individual inflation and deflation of said channels with liquid or air effects the bending of the tip of the device.

In some embodiments provided herein, the device comprises a hydraulic articulation and propulsion mechanism. In some embodiments, the device may be driven by an articulation movement powered by hydraulic actuated flexible cylinders and/or rods to bend the tip of the device. For example, three hydraulically powered flex rods may enable the instrument to bend when individually extended/retracted with non-compressible fluid. The proximal/first controllably expandable element and the distal/second controllably expandable element, e.g., balloons, may independently inflate and deflate to fasten the device to the interior walls of the GI tract while a propulsion mechanism utilizing a hydraulic or mechanically powered actuator in between the elements pushes and pulls the device through the intestines. Mechanisms including hydraulic actuators, lead screws, cable assemblies can be used for the propulsion movement.

In some embodiments provided herein, the device comprises a hydraulic actuator in between the proximal/first controllably expandable element and the distal/second controllably expandable element of the device, and engagement of the hydraulic actuator effects the sliding movement between the outer member and the inner member of the device. In other embodiments, mechanical actuators like lead screws or cable assemblies can be used instead. In some embodiments, the device further comprises a plurality of flexible rods running longitudinally through the device, and individual extension and retraction of said rods with non-compressible fluid effects the bending of the tip of the device.

In some embodiments provided herein, the device comprises a cable-driven actuator in between the proximal/first controllably expandable element and the distal/second controllably expandable element of the device, and engagement of the cable-driven actuator effects the sliding movement between the outer member and the inner member of the device. In other embodiments, hydraulic actuators or lead screws can be used instead. In some embodiments, the device further comprises additional cables running longitudinally through the device, the distal ends of said cables fixed in the tip of the device. The cables are coupled with a plurality of motor-pulley systems, and individual pulling and pushing of said cables by the motor-pulley systems effects the bending of the tip of the device.

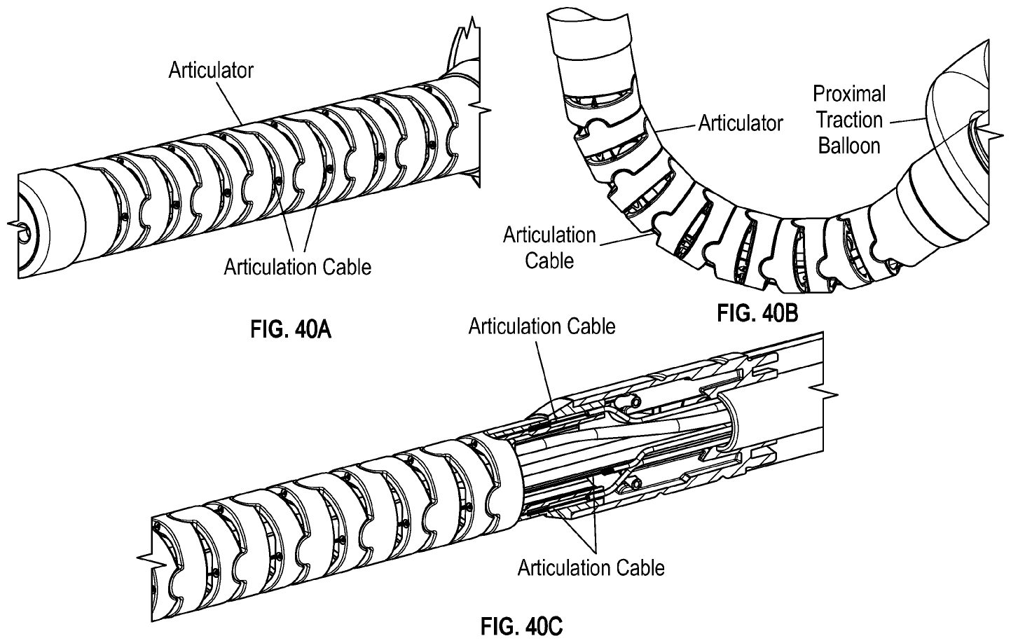

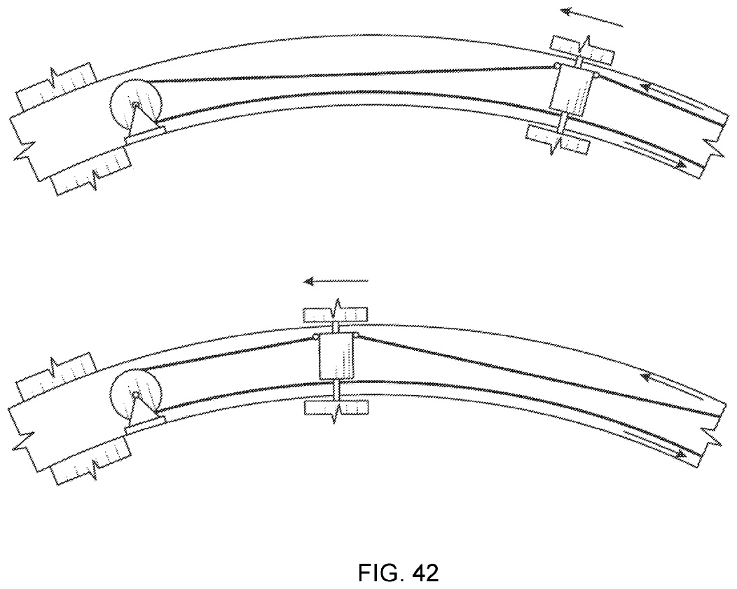

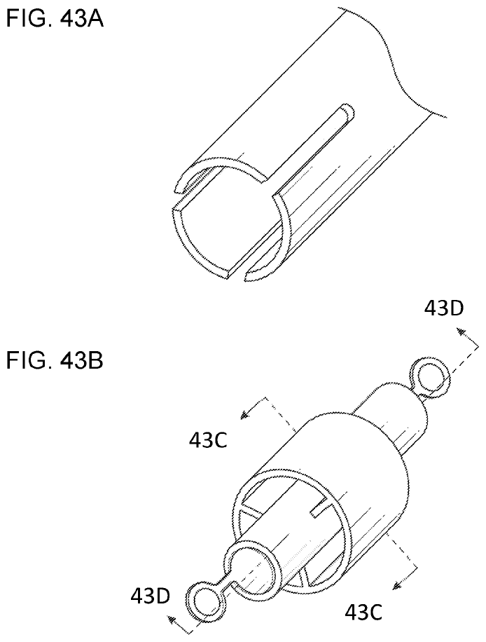

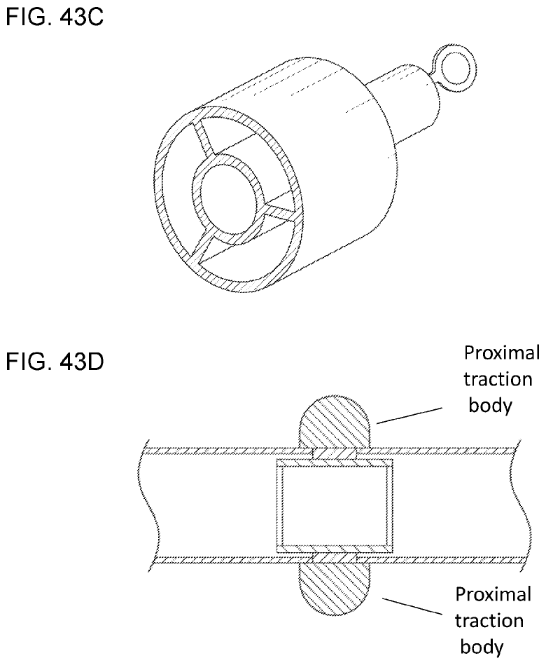

In some embodiments provided herein, the device further comprises a plurality of closed loop cables running longitudinally through the device, the distal ends of said cables fixed in the tip of the device. The cables are coupled with a plurality of motor-pulley systems, and individual pulling and pushing of said cables by the motor-pulley systems effects the bending of the tip of the device for articulation, as shown in FIG. 39. A flexible housing unit surrounds the cable assembly to contain the articulation mechanism, as shown in FIGS. 40A-40C. A pulley system can also be used to articulate the tip of the device and/or to effect the relative movement of the radially controllably expandable elements (e.g., the traction balloons), for instance, as shown in FIGS. 40-42.

In some embodiments provided herein, the device further comprises one or more closed loop cables running longitudinally through the device, the distal ends of said cables fixed between the distal and the proximal radially expandable elements. In some embodiments, the proximal radially expandable element is connected to the close loop cable. The cables can be coupled with a plurality of motor-pulley systems, and individual pulling and pushing of said cables by the motor-pulley systems effects the movement of the radially expandable elements.

In some embodiments provided herein, the device comprises a three phase servo motor actuator. In this embodiment, linearly oriented coils are energized in sequence to propel the balloon mechanism forwards and backwards. The device further comprises a bidirectional magnet installed on the balloon mechanism in order to integrate with the magnetic linear actuator.

In some aspects, provided herein is a device configured to move within a body cavity, said device comprising: an outer member comprising a distal end, a proximal end, a lumen between the distal end and the proximal end, and a proximal/first controllably expandable element; an inner member slidably disposed in the lumen of the outer member, wherein the inner member comprises a distal end, a proximal end, and a distal/second controllably expandable element; a connector that connects the outer member and the inner member; and an actuating member comprising a plurality of balloons (e.g., pressure balloons, or axially compliant balloons), a plurality of bellows or unit bellows, and/or a plurality of pressure chambers, wherein the actuating member is capable of effecting sliding movement between the outer member and the inner member, thereby alternating extensions and retractions of a distance between the proximal/first and distal/second controllably expandable elements, wherein the proximal/first and distal/second controllably expandable elements are capable of expanding radially outwardly to engage a wall of a body cavity. In any of the preceding embodiments, the actuating member is also capable of effecting articulation of the distal portion of the inner tube in a direction transverse to the longitudinal axis of the body portion of the inner tube, for example, via selective or preferential inflation and/or deflation of one or more of the plurality of balloons, the plurality of bellows or unit bellows, and/or the plurality of pressure chambers.

In some embodiments, provided herein is a device configured to move within a body cavity, the device comprising: a) an elongated support; b) a proximal radially expandable element and a distal radially expandable element positioned along the length of the elongated support, optionally wherein the radially expandable elements are independently controllably expandable, wherein the radially expandable elements comprise outer surfaces configured to frictionally engage a wall of a body cavity, and wherein the distal radially expandable element is fixed relative to the elongated support; and c) a locomotion system comprising: i) a proximal locomotion element having a part that is fixed relative to the proximal radially expandable element and slidable along the length of the elongated support, such that the proximal radially expandable element is slidable along the length of the elongated support, and ii) a distal locomotion element having a part fixed relative to the elongated support, wherein the locomotion system effects sliding movement of the proximal radially expandable element along the length of the elongated support, thereby effecting relative movement between the radially expandable elements and movement of the device within the body cavity.

In some embodiments, the locomotion system comprises a controllably expandable structure configured to expand or contract longitudinally. In some aspects, the controllably expandable structure is distal to the proximal/first controllably expandable element, wherein longitudinal expansion and/or contraction of the controllably expandable structure effects a longitudinal movement of the proximal/first controllably expandable element relative to the distal/second controllably expandable element. In some aspects, the controllably expandable structure is proximal to the proximal/first controllably expandable element, wherein longitudinal expansion and/or contraction of the controllably expandable structure effects a longitudinal movement of the proximal/first controllably expandable element relative to the distal/second controllably expandable element. In some aspects, the device comprises two controllably expandable structures, one of which is distal to the proximal/first controllably expandable element while the other one is proximal to the proximal/first controllably expandable element, wherein coordinated longitudinal expansion and/or contraction of the two controllably expandable structures effects a longitudinal movement of the proximal/first controllably expandable element relative to the distal/second controllably expandable element. In some aspects, the controllably expandable structure is distal to the distal/second controllably expandable element, wherein longitudinal expansion and/or contraction of the controllably expandable structure effects a longitudinal movement of the proximal/first controllably expandable element relative to the distal/second controllably expandable element. In some aspects, the controllably expandable structure is proximal to the distal/second controllably expandable element, wherein longitudinal expansion and/or contraction of the controllably expandable structure effects a longitudinal movement of the proximal/first controllably expandable element relative to the distal/second controllably expandable element. In some aspects, the device comprises two controllably expandable structures, one of which is distal to the distal/second controllably expandable element while the other one is proximal to the distal/second controllably expandable element, wherein coordinated longitudinal expansion and/or contraction of the two controllably expandable structures effects a longitudinal movement of the proximal/first controllably expandable element relative to the distal/second controllably expandable element. In some aspects, the controllably expandable structure is between the proximal/first controllably expandable element and the distal/second controllably expandable element, wherein longitudinal expansion and/or contraction of the controllably expandable structure effects a longitudinal movement of the proximal/first controllably expandable element relative to the distal/second controllably expandable element.

In any of the preceding embodiments, the device can further comprise a plurality of controllably expandable structures between the proximal/first controllably expandable element and the distal/second controllably expandable element, wherein expansion and/or contraction of the plurality of controllably expandable structures effects a longitudinal movement of the proximal/first controllably expandable element relative to the distal/second controllably expandable element. In some embodiments, the plurality of controllably expandable structures form a helix. In any of the preceding embodiments, expansion and/or contraction of the plurality of controllably expandable structures effects a rotational movement of the proximal/first or distal/second controllably expandable element relative to each other. In some aspects, the proximal/first or distal/second controllably expandable element is in a contracted or deflated state during the rotational movement. In any of the preceding embodiments, the device can further comprise two, three or more controllably expandable structures. In any of the preceding embodiments, expansion and/or contraction of the plurality of controllably expandable structures effects articulation of a distal portion of the device in a direction transverse to the longitudinal axis of the elongated support.

In any of the preceding embodiments, the controllably expandable structures can comprise one or more compliant balloon and/or one or more semi-compliant balloon. In any of the preceding embodiments, the controllably expandable structures can comprise one or more bellows, e.g., a compliant bellows. In some aspects, the plurality of controllably expandable structures comprise two or more pressure balloons. In some aspects, the plurality of controllably expandable structures comprise a pressure balloon, a pressure chamber, or combinations thereof. In some aspects, the plurality of controllably expandable structures comprise three or four pressure balloons. In some embodiments, the plurality of controllably expandable structures comprise three or four pressure chambers. In any of the preceding embodiments, a subset of the plurality of controllably expandable structures can be configured to selectively inflate and/or deflate, thereby effecting articulation of the distal/second controllably expandable element in a direction transverse to the longitudinal axis of the elongated support.

In any of the preceding embodiments, the device can further comprise a plurality of controllably expandable structures distal to the distal/second controllably expandable element, wherein a subset of the plurality of controllably expandable structures are configured to selectively inflate and/or deflate, thereby effecting articulation of the distal end of the device in a direction transverse to the longitudinal axis of the elongated support.

One or more traction-motion element can be used, in place of or in addition to one or more controllably expandable element of any of the embodiments of the device or method disclosed herein. For example, the proximal/first controllably expandable element may be a traction-motion element disclosed herein. In other examples, the distal/second controllably expandable element may be a traction-motion element disclosed herein. In yet other examples, both the proximal/first controllably expandable element and the distal/second controllably expandable element may be a traction-motion element disclosed herein. The traction-motion element(s) may provide an actuating/motion mechanism in addition to the actuating/motion mechanism(s) of any of the embodiments of the device or method disclosed herein.

In some embodiments, the locomotion system comprises two longitudinally expandable elements. In some embodiments, the locomotion system comprises a proximal longitudinally expandable element and a distal longitudinally expandable element. In some embodiments, the longitudinally expandable elements are independently controllably expandable. In some embodiments, the longitudinally expandable elements each comprises a structure independently selected from the group consisting of a compliant balloon, a semi-compliant balloon, a pressure balloon, and a bellow.

In some embodiments, the locomotion system comprises a pulley system. The pulley system effects sliding movement of the proximal radially expandable element along the length of the elongated support, thereby effecting relative movement between the radially expandable elements and movement of the device within the body cavity. In some embodiments, the pulley system comprises a proximal floating element. In some embodiments, the proximal floating element is fixed relative to the proximal radially expandable element and slidable along the length of the elongated support, such that the proximal radially expandable element is slidable along the length of the elongated support. In some embodiments, the pulley system comprises a distal wheel fixed relative to the elongated support. In some embodiments, the pulley system comprises a cable connected to the proximal floating element and engaging the distal wheel, such that the cable is configured to pull the proximal floating element in the distal or proximal direction. In some embodiments, the pulley system comprises a closed loop cable.

Exemplary body cavity navigation devices are described US 2021/0345862 entitled “Devices and Systems for Body Cavities and Methods of Use,” and WO 2021/173818 entitled “Devices and Systems for Body Cavities and Methods of Use,” the disclosures of which are incorporated herein by reference in their entireties.

Reference is now made to the figures, which describe certain elements or aspects of multiple embodiments of the present disclosure. The drawings are provided for illustrative purposes only and are not meant to be limiting.

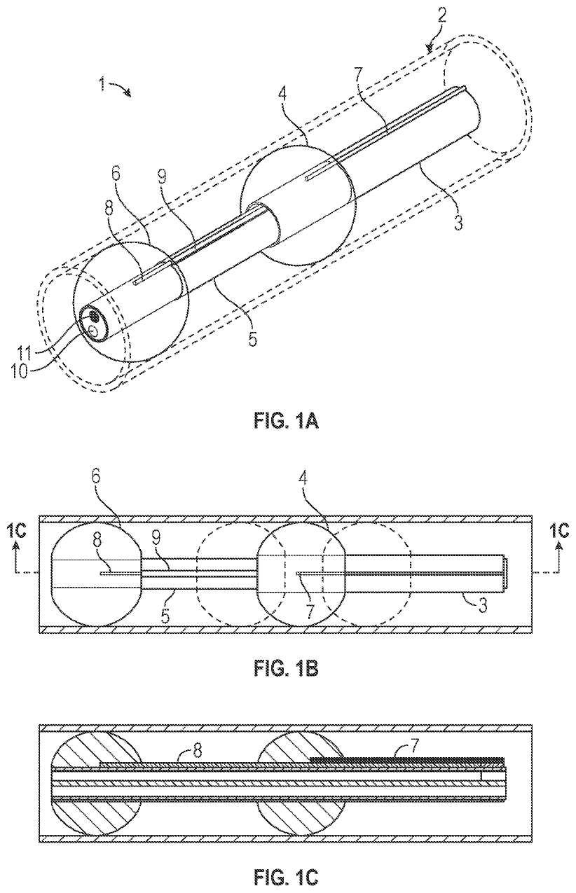

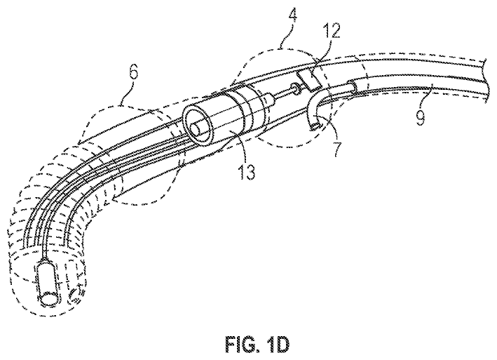

FIGS. 1A-1D show an exemplary device disclosed herein comprising two controllably expandable elements. As shown in FIG. 1A, a distal portion of device 1 may be placed inside a body cavity 2 such as a gastrointestinal (GI) tract of a subject. The outer tube 3 (e.g., an overtube) comprises a distal end, a proximal end, a lumen between the distal end and the proximal end, and a proximal/first controllably expandable element 4 on an outer surface of the outer tube. The proximal/first controllably expandable element can be a balloon capable of expanding radially outwardly to engage a wall of the body cavity 2. The inner tube 5 is slidably disposed in the lumen of the outer tube 3, and comprises a distal end and a proximal end. The inner tube 5 further comprises, on an outer surface of the inner tube, a distal/second controllably expandable element 6. The distal/second controllably expandable element can be a balloon capable of expanding radially outwardly to engage a wall of the body cavity 2. The proximal/first and distal/second controllably expandable elements 4 and 6 may be controllably inflated or deflated through medium channels 7 and 8, respectively. The medium in the channels may be a gas, a liquid, or a combination thereof (e.g., a vapor), and the channels may be protected by a shrink tube 9. The inner tube 5 may comprise one or more working channel 10 and/or one or more chamber or channel for camera 11. When the controllably expandable elements 4 and 6 are expanded, they may engage the body cavity wall at different positions, as shown in the side view and a cross-section of the distal portion of device inside the body cavity in FIG. 1B and FIG. 1C, respectively.

In any of the embodiments herein, the support can be in the form of a tubular structure, such as inner tube 5 shown in FIGS. 1A-1D, and a radially expandable structure (e.g., the proximal/first controllably expandable element 4 such as a proximal traction balloon) can be disposed on a ring (such as outer tube 3 shown in FIGS. 1A-1D) around the support.

As shown in FIG. 1D, a connecting mechanism or connector 12 connects the outer tube 3 and the inner tube 5. An actuating mechanism or actuator 13 capable of effecting sliding movement between the outer tube 3 and the inner tube 5 is provided to alternate extensions and retractions of a distance between the balloon 4 and 6 along the length of the body cavity. The inner tube 5 may be inserted into the outer tube 3 under the condition that air is exhausted from the balloons 4 and 6 to deflate the balloons. The medium channel 7 providing a medium for inflating and/or deflating the balloon 4 is also shown, and the medium channel may be protected by the shrink tube 9. In preferred embodiments, the inner tube and the outer tube are pre-assembled, with the inner tube slidably placed inside the outer tube, before the distal portion of the device is placed inside the body cavity. In preferred embodiments, the inner tube and the outer tube are pre-assembled and connected to each other via a connecting mechanism, with the inner tube slidably placed inside the outer tube, before the distal portion of the device is placed inside the body cavity. Thus, there is no need for an operator to insert the inner tube through the outer tube during operation of the device.

The distal end of the device may be placed within the body cavity at an initial position proximal to the operator. In a retrograde (anal) approach, the initial position may be at a position in the rectum or colon, such as at the sigmoid colon, the descending colon, the transverse colon, or the ascending colon. In an anterograde approach, the initial position may be at a position in the esophagus, stomach, or small intestine, such as at the duodenum. Both balloons 4 and 6 may be completely deflated or in a less inflated state when placed at the initial position, and/or when the device is being placed at the initial position, for example, for ease of operation and patient comfort.

As an initial step, a remote control may be operated to supply a medium such as air from a pump outside the body of the subject to the balloon 4 attached in the distal end of the outer tube 3, thus inflating the balloon and fixing the balloon at the initial position. Consequently, the outer tube 3 is fixed to the initial position in the body cavity, such as the colon.

While the inflation state of the balloon 4 is maintained, sliding movement between the outer tube 3 and the inner tube 5 is actuated and optionally controlled by a control unit outside the body of the subject, to insert the inner tube 5 into a deeper part (e.g., more distal to the operator, e.g., the small intestine) of the body cavity, while the balloon 6 is deflated or in a less inflated state to permit the sliding movement. Consequently, the distance between the balloon 4 and 6 along the length of the body cavity becomes greater. After the inner tube 5 is inserted deeper by a distance, a remote control may be operated to supply a medium such as air from a pump outside the body of the subject to the balloon 6 attached in the distal end of the inner tube 5, thus inflating the balloon 6 and fixing the balloon at a more distal position. Consequently, the inner tube 5 is fixed to the more distal position, such as the small intestine.

The distance of the inner tube 5 movement may be a predetermined distance, or may be manually or automatically adjusted during operation. For example, a pressure sensor at the tip of the device may feed a detected pressure signal to a control unit outside the patient body, if the pressure sensed is over a certain threshold indicating stretching of the body cavity wall, thus the distance of inner tube advancement may be reduced or the articulation of the tip of the device may be adjusted, in order to eliminate or reduce stretching.

While the inner tube 5 is fixed at the more distal position, a remote control may be operated to exhaust air from the balloon 4, which becomes deflated or less inflated so as to permit movement of the outer tube within the body cavity to a more distal position. Sliding movement between the outer tube 3 and the inner tube 5 is once again actuated and optionally controlled by a control unit outside the body of the subject, to move the outer tube 3 more distally into the body cavity, while the balloon 6 is inflated and balloon 4 is deflated or less inflated. Consequently, the distance between the balloon 4 and 6 along the length of the body cavity becomes smaller, and both balloons are now positioned at a more distal portion of the body cavity compared to the initial position that is more proximal to the operator. A remote control may be operated to supply a medium such as air from a pump outside the body of the subject to the balloon 4 attached in the distal end of the outer tube 3, thus inflating the balloon and fixing the balloon at the more distal position. While the inflation state of the balloon 4 is maintained, sliding movement between the outer tube 3 and the inner tube 5 is once again actuated to insert the inner tube 5 into a deeper part of the body cavity, while the balloon 6 is deflated or in a less inflated state to permit the sliding movement. Operation steps described above can be repeated to advance a distal end of the device into deeper parts, such as from colon to the small intestine, from ileum to jejunum, from jejunum to duodenum, or from duodenum to stomach.

In an alternative initial step, a remote control may be operated to supply a medium such as air from a pump outside the body of the subject to the balloon 6 attached in the distal end of the inner tube 5, thus inflating the balloon and fixing the balloon at the initial position. Consequently, the inner tube 5 is fixed to the initial position in the body cavity, such as the colon.

While the inflation state of the balloon 6 is maintained, sliding movement between the outer tube 3 and the inner tube 5 is actuated and optionally controlled by a control unit outside the body of the subject, to advance the outer tube 3 into a deeper part (more distal to the operator) of the body cavity, while the balloon 4 is deflated or in a less inflated state to permit the sliding movement. After the outer tube 3 is advanced deeper by a distance, a remote control may be operated to supply a medium such as air from a pump outside the body of the subject to the balloon 4 attached in the distal end of the outer tube 3, thus inflating the balloon 4 and fixing the balloon. Consequently, the outer tube 3 is fixed to a position distal to its initial position. The distance between the balloon 4 and 6 along the length of the body cavity also becomes smaller.

The distance of the outer tube 3 movement may be a predetermined distance, or may be manually or automatically adjusted during operation.

While the outer tube 3 is fixed at the more distal position, a remote control may be operated to exhaust air from the balloon 6, which becomes deflated or less inflated so as to permit movement of the inner tube within the body cavity to a more distal position. Sliding movement between the outer tube 3 and the inner tube 5 is once again actuated and optionally controlled by a control unit outside the body of the subject, to move the inner tube 5 distally into the body cavity, while the balloon 4 remains inflated and balloon 6 is deflated or less inflated. Consequently, the distance between the balloon 4 and 6 along the length of the body cavity becomes greater. When balloon 6 reaches a more distal destination, a remote control may be operated to supply a medium such as air from a pump outside the body of the subject to the balloon 6 attached in the distal end of the inner tube 5, thus inflating the balloon and fixing the balloon at the more distal position. While the inflation state of the balloon 6 is maintained, sliding movement between the outer tube 3 and the inner tube 5 is once again actuated to advance the outer tube 3 into deeper part of the body cavity, while the balloon 4 is deflated or in a less inflated state to permit the sliding movement. Operation steps described above can be repeated to advance a distal end of the device into deeper parts, such as from colon to the small intestine, from ileum to jejunum, from jejunum to duodenum, or from duodenum to stomach.

In any of the preceding embodiments, the device disclosed herein may also be operated to move from a more distal part of a body cavity to a more proximal part of the body cavity. In other words, the device disclosed herein may also be operated to move backwards. In any of the preceding embodiments, the device disclosed herein may move forward and backward in the body cavity, in any suitable combination or order, according to medical needs.

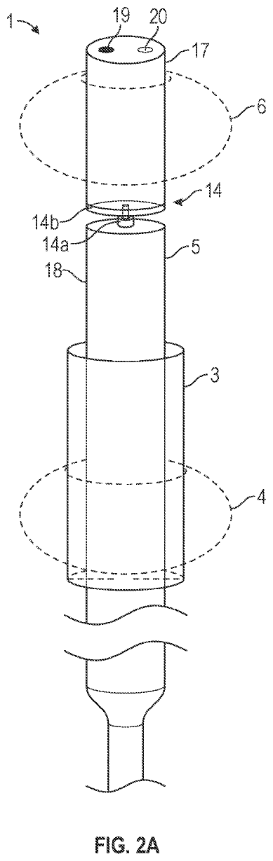

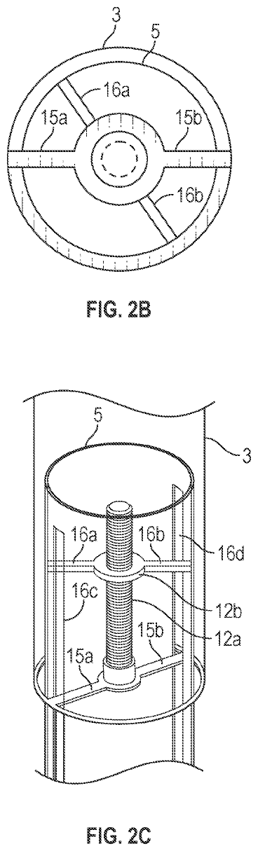

FIG. 2A shows an exemplary device disclosed herein comprising an inner tube 5 and an outer tube 3, two controllably expandable elements (e.g., balloons) 6 and 4 on the inner tube and the outer tube, respectively, and an articulation mechanism 14.

FIGS. 2B and 2C show an exemplary device that further comprises a screw/nut connector 12 and actuating mechanism 13. As shown in FIG. 2C, the device 1 comprises a screw 12a and a nut 12b, for the inner and outer tubes to move relative to each other. The screw 12a is inside the inner tube but connected to the outer tube via a motor 13, such as a stepper motor. The motor may be connected to a proximal portion of the outer tube as shown in FIG. 2A. The motor connects to the outer tube 3 via two arms 15a and 15b, which as shown in FIG. 2B are fixed onto the outer tube. The inner tube 5 connects to the nut 12b which is fixed onto the inner tube via two arms 16a and 16b. The rotation of the screw 12a enables the nut 12b and the inner tube 5 to move along the outer tube 3. When balloon 4 keeps the outer tube 3 (and the arms connecting to the motor) stationary, the inner tube 5 can move forward when the screw/nut moves in one direction and backward when the screw/nut moves in the opposite direction. When balloon 6 keeps the inner tube 5 (and the nut fixed thereto) stationary, the outer tube 3 can move forward and backward as well. FIG. 2C shows two longitudinal slits 16c and 16d on opposite walls of the inner tube 5. The two arms 15a and 15b extending from the motor 13 pass through the slits and are fixed onto the outer tube 3.

Referring again to FIG. 2A, the inner tube 5 comprises two portions, a distal end portion 17 and a proximal body portion 18. The distal end portion has an opening 19 for a camera and an opening 20 for air and/or water, e.g., as an opening for an irrigation and/or suction channel. The proximal end section of the inner tube distal end portion 17 comprises the base 14b which is connected to the body portion 18 via the motor 14a.

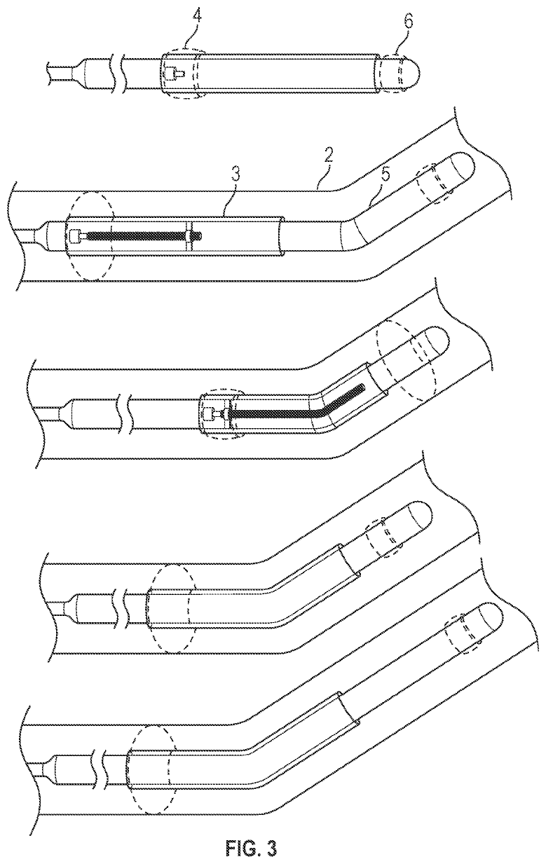

As shown in FIG. 3, both balloons 4 and 6 are deflated before and when the device is placed inside the GI tract. The balloons can be circumferentially wrapping around the inner and/or outer tubes. After the device reaches an initial position, balloon 4 is inflated to anchor the outer tube 3 on the bowel wall 2. The outer tube and its balloon are kept relatively stationary to the bowel wall part engaged by the inflated balloon, while the inner tube 5 moves forward to reach its distance. During the movement of the inner tube, an articulation mechanism such as the motor 14a and the base 14b shown in FIG. 2A may effect the articulation of a distal tip of the inner tube such that the inner tube may make turns following how the GI curves. Thus, the articulation mechanism may reduce or minimize stretching of the body cavity wall due to the movement of the inner tube. Both the inner and outer tubes, including the screw shown in the figure, may be made of flexible material. In addition, if relatively more rigid material is required, the outer tube including the screw may be made sufficiently small. Once the inner tube 5 reaches a more distal destination, it is anchored on the bowel wall 2 by inflating the balloon 6. Then, the outer tube 3 follows by moving forward over the inner tube while the balloon 4 is deflated. FIG. 3 shows the balloon at the proximal end of the outer tube. However, it is to be understood that the balloon may be provided along the entire length of the outer tube. Similarly, it is not necessary that the balloon on the inner tube be at the very distal end; it may be provided along the length of the inner tube at a suitable position to permit the alternating extensions and retractions of the device. Once the outer tube is in place (more distal in the GI tract compared to the initial position), it is anchored on the bowel wall by inflating the balloon 4. The balloon 6 on the inner tube is then deflated and moves forward to an even more distal position. The process continues until it reaches a destination, such as a more distal destination in the GI tract, e.g., the small intestine.

In any of the embodiments disclosed herein, the inflated balloon may comprises a wavy, ribbed, and/or saw tooth shaped or patterned outer surface that is configured to frictionally engage the body cavity wall. In some embodiments, when the balloon is deflated, the wavy, ribbed, and/or saw tooth shapes or patterns on the outer surface shrink down, effectively folding up when the balloon is not frictionally engaged to the body cavity wall.

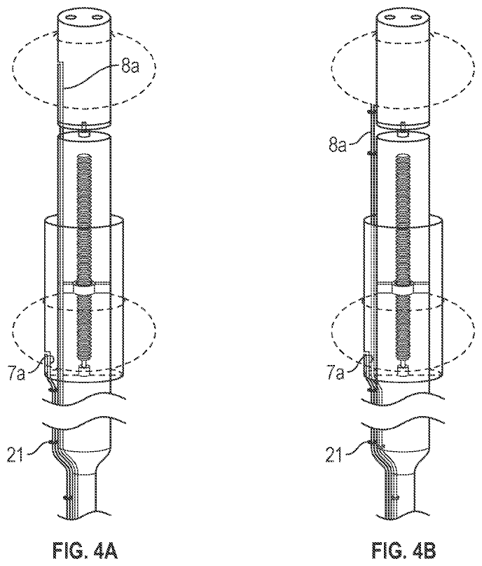

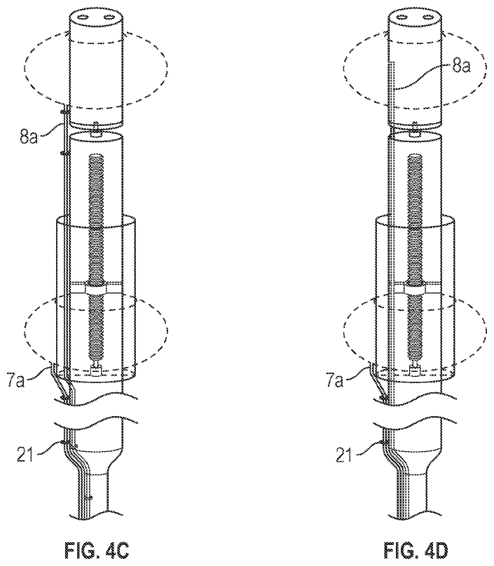

FIGS. 4A-4D show various configurations of the medium channels or tubing controlling the inflation and/or deflation of the balloons. FIG. 4A shows a medium channel or tubing 8a (connecting the balloon 6 to a medium source) inside the inner tube 5, and a medium channel or tubing 7a (connecting the balloon 4 to a medium source) inside the outer tube 3. FIG. 4B shows a medium channel or tubing 8a along the outside of the inner tube 5 and partially inside the outer tube 3, and a medium channel or tubing 7a inside the outer tube 3. FIG. 4C shows a medium channel or tubing 8a along the outside of the inner tube 5 and partially inside the outer tube 3, and a medium channel or tubing 7a along the outside of the outer tube 3. FIG. 4D shows a medium channel or tubing 8a inside the inner tube 5, and a medium channel or tubing 7a along the outside of the outer tube 3. The medium channel or tubing may be tethered to the outer or inner tube by tethering mechanisms 21.

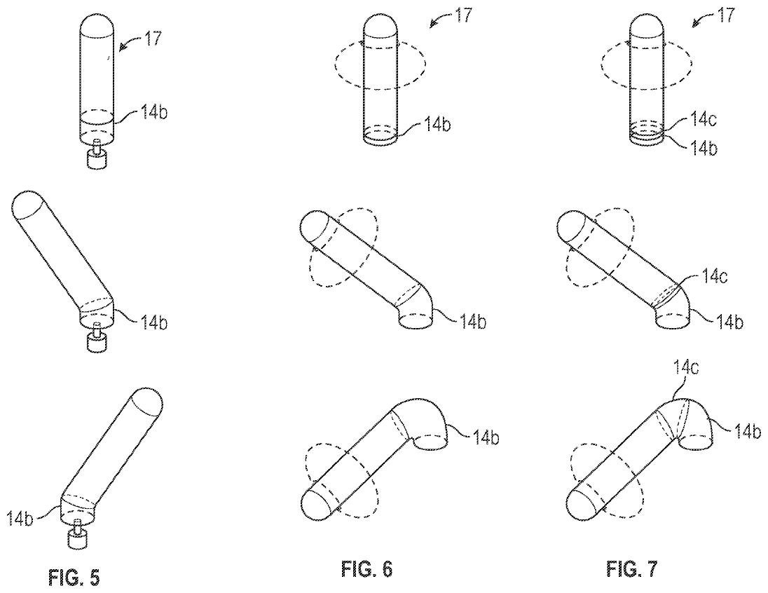

FIG. 5 shows by combining rotation of the base 14b and tilting the tip 17, the tip portion of the inner tube is capable of guiding the inner tube to move in various directions. The base 14b is a flexible conduit-like structure, except it is asymmetrical and has a hinge at one side. As shown in FIG. 6, the chamber of the round base 14b can maintain an angle from 0 degree to 180 degree at the hinge by inflating the base chamber, in order to effect articulation of the distal end portion 17 of the inner tube. Another chamber 14c can be provided on top of the base 14b to share the same hinge with the base, as shown in FIG. 7.

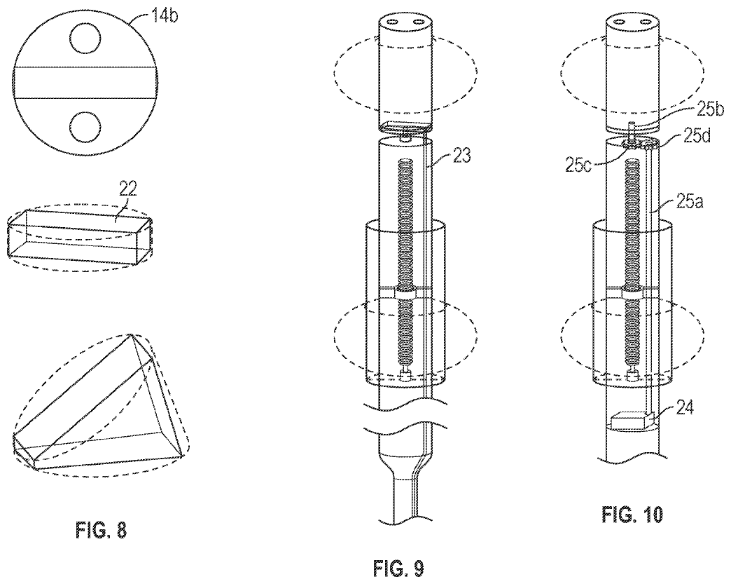

FIG. 8 shows inside the round base 14b, there is a thin cuboid shaped chamber 22 that can be inflated asymmetrically to a triangular shape, thereby inflating the round base to a desired angle. FIG. 9 shows a medium channel or tubing 23 (e.g., the medium can be a gas, a liquid, or a mixture thereof such as a vapor) going through the inner tube body and connect to the round base via a flexible conduit. FIG. 10 shows a servo motor 24 can be placed proximal to the first stepper motor 13 for the screw/nut and connected to the round base with a stiff thin wire 25a that can accurately transmit the servo motor's rotation to a pin 25b on the round base via gears 25c and 25d.

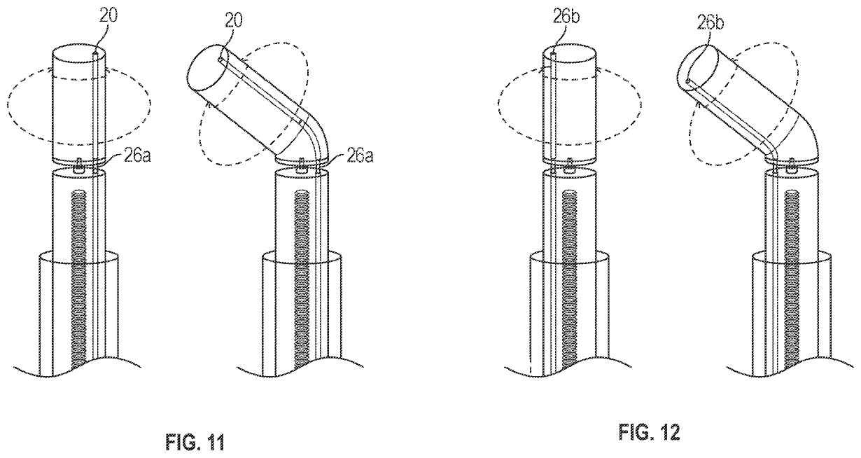

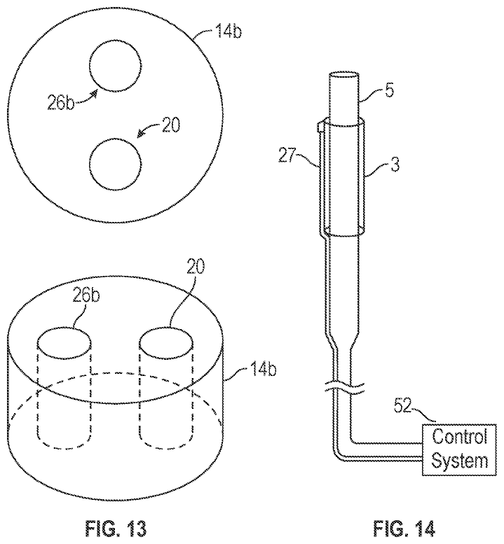

FIG. 11 shows the water/air/suction channel 20 is a channel traversing the whole inner tube from the proximal inner tube, round base to the distal inner tube. There is a flexible tube 26a that is fixed to the proximal end of the channel at the distal (tip) section of the inner tube and end freely in the air channel of the inner tube body but fits tightly in the air channel of the inner tube body to maintain a seal. The flexible tube traverses the round base down to the inner tube body at a length that is long enough to still remain in the tube body's air channel when the round base is inflated to its largest angle at the hinge and when the round base rotates up to 180 degree to both directions (clockwise and counter-clockwise). FIG. 12 shows the optical fiber or wire of a camera can traverse the entire inner tube and/or traverse the round base. FIG. 13 shows the camera channel 26b and water/air/suction channel 20 pass the base 14b through an air-sealed tunnel to ensure that the base is air sealed.

FIG. 14 shows a guidewire 27 attached to the outer tube distally and to the inner tube proximally, for example, as a carrier system that allows other mechanisms to feed over the guidewire and be delivered to a desired location. The device may further comprise the control unit or system 52.

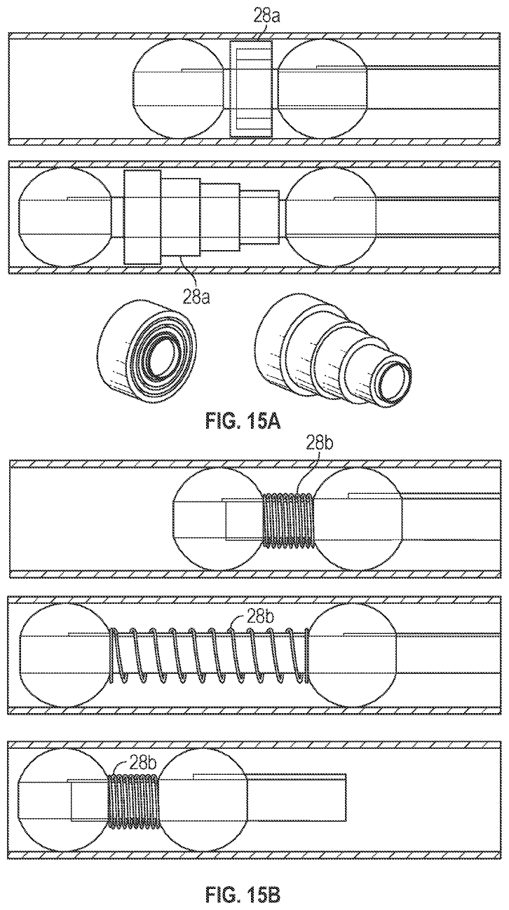

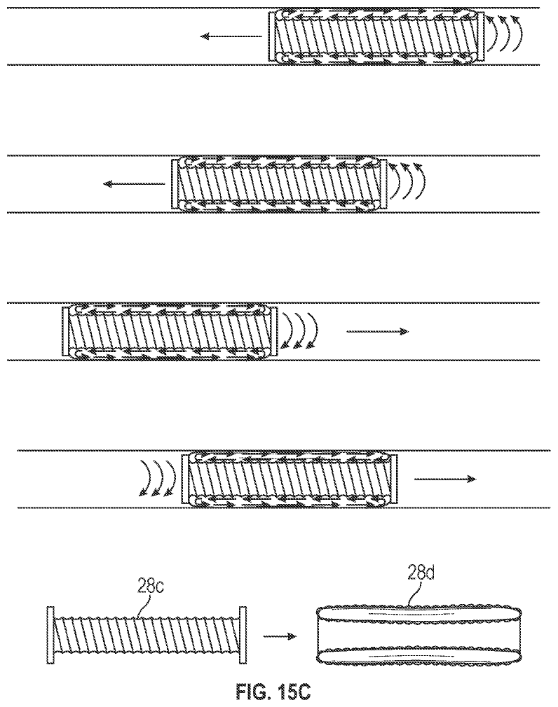

The device may be driven by an actuating mechanism based on one or more controllably expandable telescoping structure. FIG. 15A shows an actuating mechanism comprising a controllably expandable telescoping structure 28a, to effect alternating extensions and retractions of a distance between the balloons 4 and 6. A controllably expandable telescoping structure may comprise a plurality of coaxial cylindrical sections which when inflated are slidable within one another. Methods of making telescoping or nested balloons are known, for example, as shown in US 2016/0114141, which is incorporated by reference in its entirety. The controllably expandable telescoping structure may comprises one or more telescoping balloons, which are collapsed or nested when no or little pressure is applied inside the balloons and expand when pressure is applied. Thus, the controllably expandable telescoping structure may be use to provide a worm-like or caterpillar type action to advance the distal portion of the device in a body passageway. By use of the telescoping structure which have a very minor length dimension compared to a traditional endoscope, tight curves in a body passageway can be easily maneuvered around.

The device may also be driven by a shape memory alloy-based actuating mechanism. FIG. 15B shows a shape memory alloy actuating mechanism 28b to effect alternating extensions and retractions of a distance between the balloons 4 and 6. The shape memory alloy actuating mechanism may comprises one or more shape memory alloy spring. The shape memory alloy has a first, relaxed state or phase (e.g., when no power is provided) and a distal/second, actuated state or phase (e.g., when a voltage is provided). When power is withdrawn, the shape memory alloy returns to its relaxed state or phase. When shaped into a spring, the transition of the shape memory alloy from the relaxed state to the actuated state causes linear motion along the axis of the spring that is applied to the mechanical interface coupling the inner tube and the outer tube. Because of its narrow profile and linear orientation, the shape memory alloy actuating mechanism may be use to provide a worm-like or caterpillar type action to advance the distal portion of the device in a body passageway.

The device may also be driven by a snake traction mechanism, such as a snake traction sleeve shown in FIG. 15C. A rotating tube 28c with threads molded into it may push the inside of the traction sleeve 28d in one direction to get motion of the assembly in the opposite direction. In some embodiments, a snake traction sleeve may be provided between an inner member and an outer member to effect the sliding movement between the outer member and the inner member.

FIG. 16 shows controllably expandable structures configured to expand or contract longitudinally, thereby effecting the sliding movement between the outer member 3 (and its balloon 4) and the inner member (not shown). The controllably expandable structures may comprise one or more compliant balloons 29a and 29b. Compliant balloon 29a is proximal to the balloon 4 while compliant balloons 29b is distal to the balloon. Compliant balloons 29a and 29b are each connected to a medium source via a channel to controllably expand or contract the balloons. As shown in FIG. 16, upper panel, when balloon 4 is expanded and engage a body cavity wall (not shown), both compliant balloons 29a and 29b are deflated and the folds of the balloons are collapsed. FIG. 16, middle panel, shows when balloon 4 is deflated (while balloon 6 is inflated to anchor onto a body cavity wall), the proximal compliant balloon 29a may be expanded, and its longitudinal expansion drives or propels the outer member 3 (and its balloon 4) to a more distal position within the body cavity. FIG. 16, lower panel, shows balloon 4 is again inflated and outer member 3 is held stationary (while balloon 6 is deflated such that the inner member can move), the distal compliant balloon 29b may be expanded in the longitudinal direction to further drive the inner member 3 (and its balloon 4) to a more distal position within the body cavity.

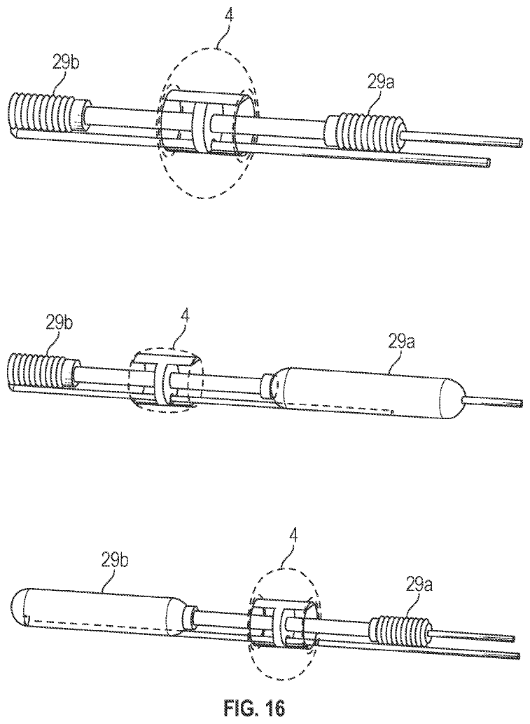

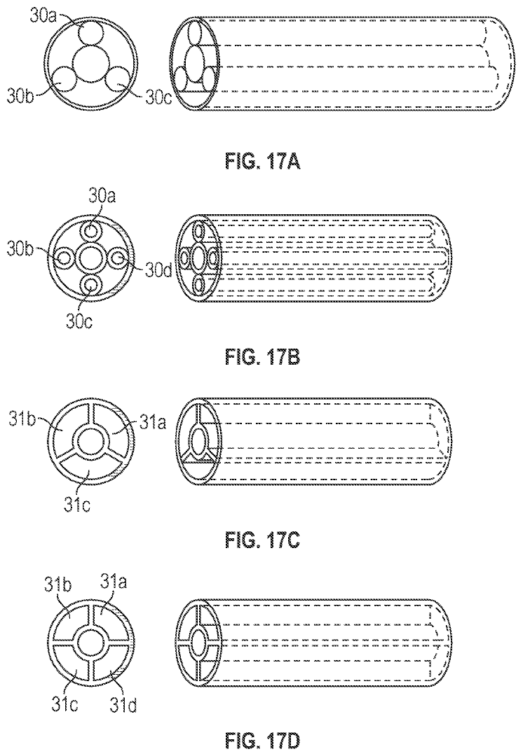

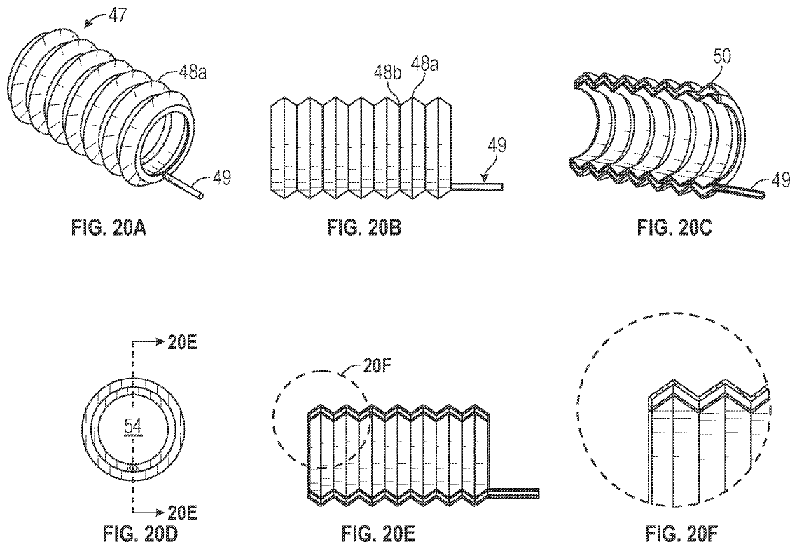

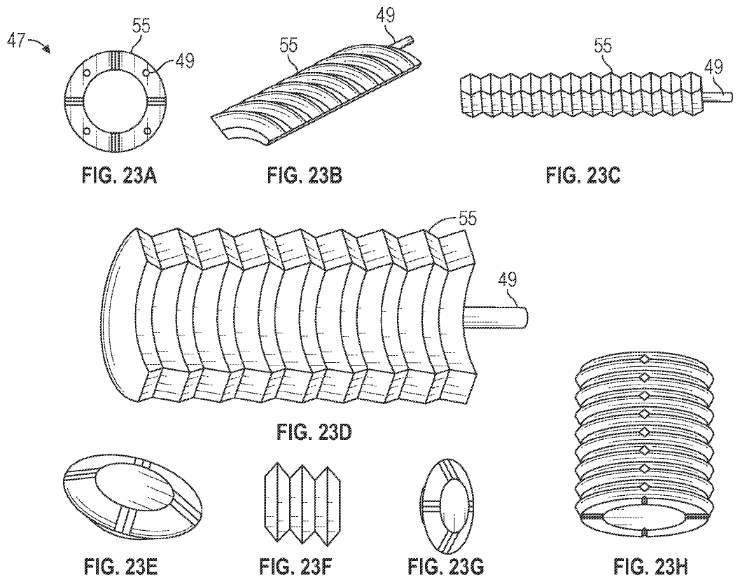

FIGS. 17A-17D show controllably expandable structures configured to expand or contract, thereby effecting the sliding movement between the outer member and the inner member and/or effecting articulation of the distal portion in a direction transverse to the longitudinal axis of the body portion of the inner tube. FIG. 17A shows the controllably expandable structures may comprise three pressure balloons 30a, 30b, and 30c, e.g., as motor balloons (e.g., for driving longitudinal movement) and/or steering balloons (e.g., for articulation of the distal tip). One or more of the pressure balloons may be selectively expanded and/or expanded to different degrees (e.g., by using different inflation pressures) such that the distal tip of the inner tube may be turned in a desired direction. FIG. 17B shows the controllably expandable structures may comprise four pressure balloons 30a, 30b, 30c, and 30d, e.g., as motor balloons (e.g., for driving longitudinal movement) and/or steering balloons (e.g., for articulation of the distal tip). FIG. 17C shows the controllably expandable structures may comprise three pressure chambers 31a, 31b, and 31c, e.g., as motor chambers (e.g., for driving longitudinal movement) and/or steering chambers (e.g., for articulation of the distal tip). One or more of the pressure chambers may be selectively expanded and/or expanded to different degrees (e.g., by using different inflation pressures) such that the distal tip of the inner tube may be turned in a desired direction. FIG. 17D shows the controllably expandable structures may comprise four pressure chambers 31a, 31b, 31c, and 31d, e.g., as motor chambers and/or steering chambers. It is to be understood that in any of the preceding embodiments, the controllably expandable structures can be bellows (e.g., as shown in FIG. 20A) instead of pressure balloons or pressure chambers, and the bellows can be assembled from a plurality of unit bellows (e.g., as shown in FIGS. 23A-23H).

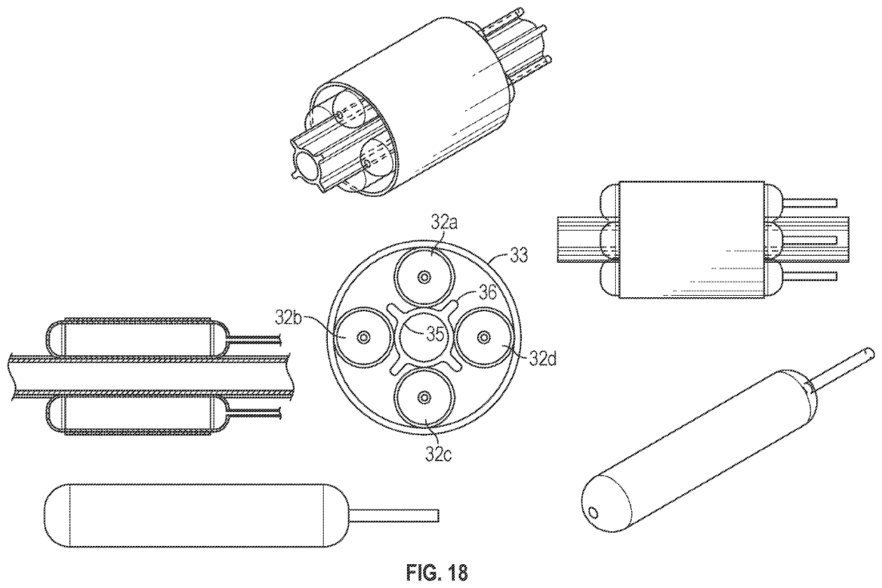

FIG. 18 shows four pressure balloons 32a, 32b, 32c, and 32d, configured to expand or contract, thereby effecting the sliding movement between the outer member 33 and the inner member 35 and/or effecting articulation of the distal portion of the inner member in a direction transverse to the longitudinal axis of the body portion of the inner member. Each of four pressure balloons may be connected to a medium channel 34 for controllably expanding one or more of the pressure balloons. The pressure balloons may be separated by ridges 36 which may serve as dividers. It is to be understood that in any of the preceding embodiments, the controllably expandable structures can be bellows (e.g., as shown in FIG. 20A) instead of pressure balloons or pressure chambers, and the bellows can be assembled from a plurality of unit bellows (e.g., as shown in FIGS. 23A-23H).

In some embodiments, the multiple balloon/bellows/channel design (e.g., as shown in FIGS. 17A-17D) and/or the unit bellows design (e.g., as shown in FIGS. 23A-23H) may be used to allow and/or control the rotation or articulation of a distal tip of the device disclosed herein, e.g., the distal portion of the inner tube. In some embodiments, the distal portion of the inner tube can form an angle from 0 degree to 180 degree relative to a body portion of the device such as a body portion of the inner tube. For example, the angle between the distal portion of the inner tube and the body portion of the device can be about 30 degrees, about 45 degrees, about 60 degrees, about 75 degrees, about 90 degrees, about 105 degrees, about 120 degrees, about 135 degrees, about 150 degrees, about 165 degrees, or about 180 degrees.

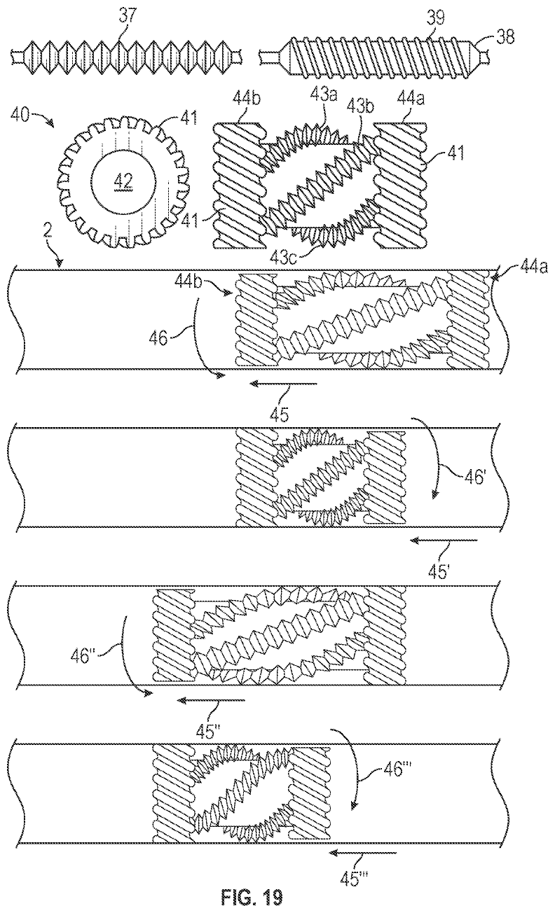

The sliding movement between the outer member 33 and the inner member 35 may also be actuated or driven by one or more controllably expandable structures, such as one or more bellows 37, one or more balloons 38 in combination with one or more springs 39 (e.g., a spring spiraling or wrapping around a balloon), as shown in FIG. 19, or any suitable combination thereof. The bellows may be compliant, such as a compliant ridge bellows. The bellows may be axially compliant and have a length that exhibits uniform axial expansion during inflation of the bellows, while being radially non-compliant in that the bellows do not expand or substantially do not expand radially during inflation. Similarly, the balloons may be compliant, such as axially compliant, and have a length that exhibits uniform axial expansion during inflation while being radially non-compliant in that the radius of each balloon exhibits little or no radial growth during inflation of the balloon. Alternatively, a balloon may be compliant but because of the spring around it, the balloon does not expand or substantially do not expand radially during inflation but is able to axially expand together with the axially expanding spring.

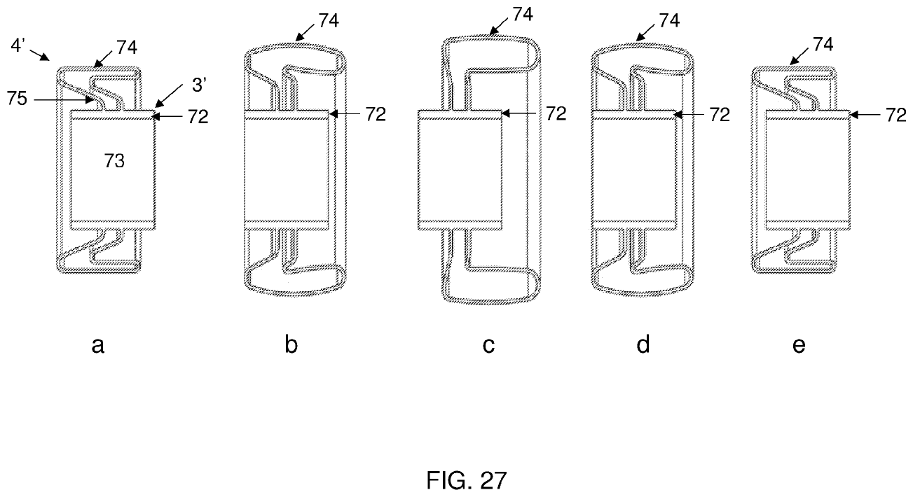

The one or more controllably expandable elements, such as the proximal/first and distal/second balloons for engaging the wall of a body cavity, may comprise a tire-like or helical gear-like structure 40 having treads 41 on an outer surface, e.g., an outside radial surface capable of frictionally engaging a wall of a body cavity (e.g., similar to outer surface 74 shown in FIG. 27). The tire-like or helical gear-like structure may have a through hole 42 having an inner surface for engaging the inner member or the outer member. The proximal/first and distal/second balloons having treads (e.g., diagonal treads) may function as traction balloons, and may be connected to each other by one or more controllably expandable structures, such as a plurality of controllably expandable structures forming a helix. As shown in FIG. 19, three controllably expandable structures 43a, 43b, and 43c may connect the proximal/first and distal/second traction balloons 44a and 44b and form a three-member helix. The controllably expandable structures may connect the outer member and the inner member at suitable structures other than the proximal/first and distal/second balloons, respectively, thus indirectly connecting the proximal/first and distal/second balloons.

The proximal/first and distal/second traction balloons 44a and 44b facilitate fixing the outer and inner members, respectively, to the body cavity wall 2 when the balloons are radially expanded. For example, traction balloon 44a may be radially expanded, and with the treads providing more traction, securely press against the body cavity wall, thereby fixing the outer member (not shown in FIG. 9) to the body cavity wall. The controllably expandable structures 43a, 43b, and 43c may be inflated while traction balloon 44b is deflated or not fully inflated (e.g., inflated but not to the extent that it may be fixed onto body cavity wall during movement). Inflation of the helical drives increases the lengths of the controllably expandable structures 43a, 43b, and 43c, thereby effecting axial movement 45 of traction balloon 44b, e.g., toward a more distal portion of the body cavity. Inflation of the helical drives also causes twisting/untwisting of the controllably expandable structures 43a, 43b, and 43c, thereby effecting rotational movement 46 of traction balloon 44b. When traction balloon 44b reaches its destination, it may be radially expanded, and with the treads providing more traction, securely press against a more distal body cavity wall, thereby fixing the inner member (not shown in FIG. 9) to the more distal body cavity wall. At this time, traction balloon 44a may be radially deflated, thereby releasing it from secure attachment to the body cavity wall and allowing axial movement 45′ of the outer member along the body cavity. During the deflation, controllably expandable structures 43a, 43b, and 43c become shorter in lengths to bring traction balloon 44a (and the outer member connected thereto) closer to the fixed traction balloon 44b. Twisting/untwisting of controllably expandable structures 43a, 43b, and 43c during the deflation also causes rotational movement 46′ of traction balloon 44a. When traction balloon 44a reaches a more distal position, it may be radially expanded again to securely press against the body cavity wall, while traction balloon 44b is deflated or not fully inflated (e.g., inflated but not to the extent that it may be fixed onto body cavity wall during movement). Controllably expandable structures 43a, 43b, and 43c are inflated, effecting axial movement 45″ and rotational movement 46″ of traction balloon 44b. When traction balloon 44b reaches an even more distal position, it may be radially expanded to securely press against an even more distal body cavity wall. At this time, traction balloon 44a is radially deflated, effecting axial movement 45′″ and rotational movement 46′″ of traction balloon 44a and bringing it closer to the fixed traction balloon 44b. The process steps may be repeated to place the device in a desired position in the body cavity, such as in the small intestine.

In any of the preceding embodiments, one or more of the controllably expandable structures, such as helical drives 43a, 43b, and 43c, may be selectively and/or preferentially inflated and/or deflated. For example, one or more of the controllably expandable structures may be inflated, while the remaining controllably expandable structure(s) is/are deflated, not inflated, or inflated to a greater or lesser degree. Alternatively, one or more of the controllably expandable structures may be deflated, while the remaining controllably expandable structure(s) is/are inflated, not deflated, or deflated to a greater or lesser degree. A suitable combination of the inflation/deflation statuses of the plurality of controllably expandable structures may be used to effect controllable and/or precise articulation of the inner member and/or the outer member, such as a distal portion of the inner member (e.g., the inner tube), thereby allowing the device to follow the curves of the body cavity during the movement. In some aspects, the controllable articulation avoids or reduces stretching of the body cavity wall, thereby avoiding or reducing discomfort during the procedure.

The one or more controllably expandable structures may comprise ridge bellows, for example, as shown in FIGS. 20A-20F. As shown in FIG. 20A (prospective view) and FIG. 20B (side view) and, bellows 47 may be an axially expandable bellows comprising a plurality of folds each having a ridge 48a and a valley 48b. The bellows may comprise an outer layer (having an outer surface and an inner surface) and an inner layer (having an outer surface and an inner surface), and the inner surface of the outer layer and the outer surface of the inner layer may sandwich a medium space 50, for example, for a gas, a liquid, or a combination thereof (e.g., a vapor). A medium may be provided to and/or withdrawn from medium space 50 through an inlet/outlet 49, in order to controllably expand and/or contract bellows 47. FIG. 20C shows a view of the bellows cut in half along the axis. The bellows may also have an internal hollow 54 that may be used to housing one or more tubing, channel, and/or wire such as electric wire. The bellows may be connected to the inner member or the outer member. For example, the bellows may house at least a portion of the inner member or the outer member in its hollow and engage the inner member or the outer member through the inner surface of the bellow's inner layer. Thus, the bellows may act as or as part of an actuating mechanism to effect the relative sliding movement between the inner and outer members. FIG. 20D shows a cross sectional view of the bellows. FIG. 20E shows the bellows cut in half along the axis, and an expanded view is provided in FIG. 20F.

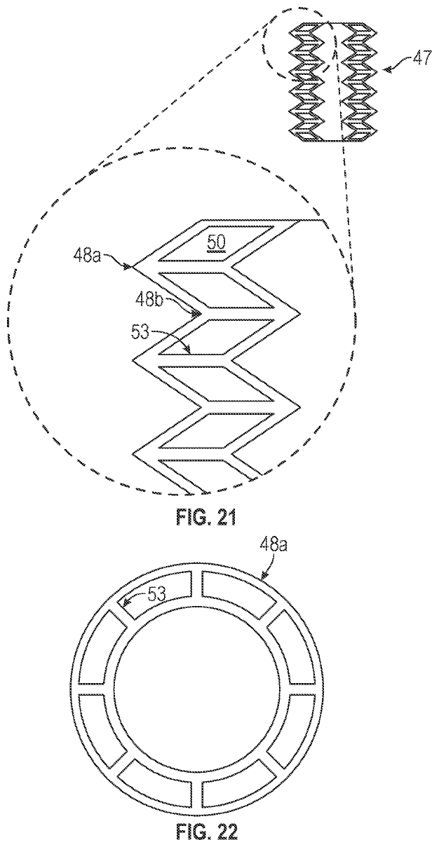

The bellows may comprise internal supports, such as one or more spokes or struts, in the medium space. The internal supports may be molded into the parts (e.g., inner and outer layers) of the bellows so that the parts stay uniform when pressurized. As shown in FIG. 21, a cross sectional view of the bellows shows ridge 48a, valley 48b, medium space 50 (such as an air or gas space), and spokes 53 connecting the outer layer and the inner layer of the bellows. Although FIG. 21 shows medium space 50 is separated into a plurality of spaces, it should be understood that the plurality of spaces are configured to be gas, liquid, or fluid communication with each other forming the medium space 50, and spokes 53 do not physically isolate the plurality of spaces. For example, a spoke may be provided between a ridge of the outer layer and the corresponding ridge of the inner layer, and/or a valley of the outer layer and the corresponding valley of the inner layer. As shown in FIG. 22, a cross sectional view, spokes 53 may serve to support the inner and outer layers or walls of the bellows relative to each other, so that the envelope of the bellows remains within a designed size when pressurized.

The bellows may comprise a plurality of unit bellows. For example, two, three, four or more unit bellows may be separately manufactured and then assembled to form a full circle of bellows, essentially as shown in FIGS. 20A-20F. For example, quarter bellows 55 having a medium channel 49 may be assembled with other quarter bellows to form bellows 47 shown in FIG. 23A (cross sectional view). Bellows 47 may have an outer diameter of about 1 inch and/or an inner diameter of about ⅝ inch. Quarter bellows 55 may be separately manufactured, as shown in FIG. 23B (prospective view showing the outer layer), FIG. 23C (side view), FIG. 23D (prospective view showing the inner layer). Identical unit bellows may be assembled, and in certain embodiments, different unit bellows may be assembled to form a full bellows. For example, unit bellows of different lengths (otherwise identical) may be assembled. In other examples, two quarter bellows and one half bellow may be assembled to form a full bellows. The unit bellows may also be separately manufactured in various parts shown in FIG. 23E, FIG. 23F, and FIG. 23G, and the parts are then assembled to form a full bellows as shown in FIG. 23H. Note each of the unit bellows in FIG. 23H may have a separate medium channel 49 such that each unit bellows may be controlled independently from other unit bellows in the same assembly.

In some aspects, the unit bellows design provides the advantage of selectively and/or preferentially inflating and/or deflating the unit bellows. For example, a full bellows may be assembled from a plurality of unit bellows, and the unit bellows may be identical or different. When the unit bellows are different, for example, in the case of two quarter bellows and one half bellows forming a full bellows, the half bellows may be selectively and/or preferentially inflated to articulate the distal portion of the inner tube to one direction. If adjustment of the bending direction is needed, one of the two quarter bellows may be selectively and/or preferentially inflated to fine tune the articulation the distal portion of the inner tube. When the unit bellows are identical, fine tuning the articulation is also possible. In the case of four quarter bellows forming a full bellows, one, two, or three of the quarter bellows may be inflated, while the remaining quarter bellows is/are deflated, not inflated, or inflated to a greater or lesser degree. Alternatively, one, two, or three of the quarter bellows may be deflated, while the remaining quarter bellows is/are inflated, not deflated, or deflated to a greater or lesser degree. A suitable combination of the inflation/deflation statuses of the unit bellows may be used to effect controllable and/or precise articulation of the inner member and/or the outer member, such as a distal portion of the inner member (e.g., the inner tube), thereby allowing the device to follow the curves of the body cavity during the movement. In some aspects, the controllable articulation and the ability to fine tune the articulation avoids or reduces stretching of the body cavity wall, thereby avoiding or reducing discomfort during the procedure.

In some embodiments, the multiple balloon/bellows/channel design (e.g., as shown in FIGS. 17A-17D) and/or the unit bellows design (e.g., as shown in FIGS. 23A-23H) may be used to allow and/or control the rotation or articulation of a distal tip of the device disclosed herein, e.g., the distal portion of the inner tube. In some embodiments, the distal portion of the inner tube can form an angle from 0 degree to 180 degree relative to a body portion of the device such as a body portion of the inner tube. For example, the angle between the distal portion of the inner tube and the body portion of the device can be about 30 degrees, about 45 degrees, about 60 degrees, about 75 degrees, about 90 degrees, about 105 degrees, about 120 degrees, about 135 degrees, about 150 degrees, about 165 degrees, or about 180 degrees.

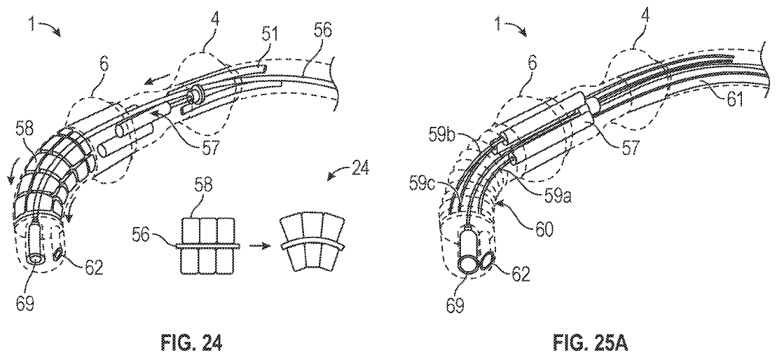

In any of the preceding embodiments, the device may comprises a soft robot articulation mechanism and/or a hydraulic propulsion or driving mechanism. For example, as shown in FIG. 24, the device 1 comprises one or more hydraulic actuator 57 in between the proximal/first controllably expandable element 4 and the distal/second controllably expandable element 6. Engagement of the inner member and outer member to the hydraulic actuator effects the sliding movement between the outer member and the inner member of the device. The device further comprises a plurality of soft, compliant fluid channels 51 running longitudinally through the device, and individual inflation and deflation of said channels with liquid or air effects the bending of the tip of the device. The device may also have a backbone 56 that flexes but does not change length, and the soft robot structures 58 allow flexing of the backbone to effect articulation of the device. As such, the distal portion of the device (such as the distal portion of the inner member) may be controlled and/or fine tuned.

In any of the preceding embodiments, the device may comprises a hydraulic articulation and/or propulsion mechanism. For example, the device 1 comprises one or more hydraulic actuator 57 in between the proximal/first controllably expandable element 4 and the distal/second controllably expandable element 6. Engagement of the inner member and outer member to the hydraulic actuator effects the sliding movement between the outer member and the inner member of the device. The device further comprises a plurality of soft, compliant fluid channels 59 running longitudinally through the device, and individual inflation and deflation of said channels with liquid or air effects the bending of the tip of the device. The device may also have a backbone 56 that flexes but does not change length, and the soft robot structures 58 allow flexing of the backbone to effect articulation of the device. As such, the distal portion of the device (such as the distal portion of the inner member) may be controlled and/or fine tuned.

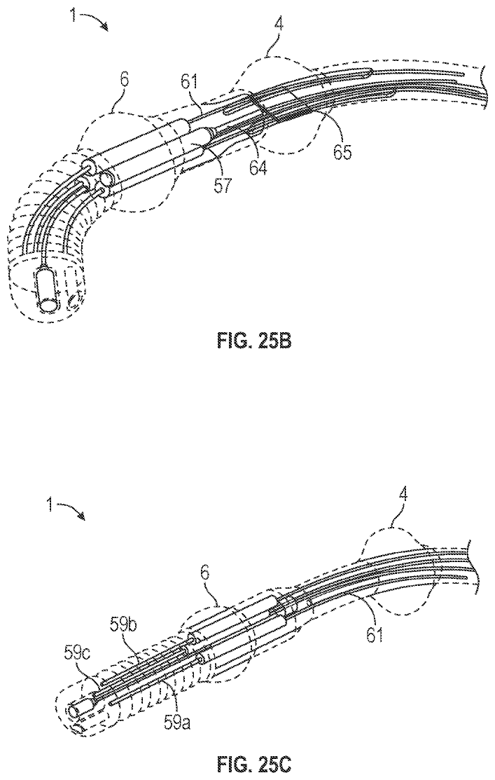

In any of the preceding embodiments, the device may comprises a hydraulic articulation and propulsion mechanism. For example, as shown in FIG. 25A, the device 1 comprises one or more hydraulic actuator 57 (connected to power via one or more hydraulic lines 61) in between the proximal/first controllably expandable element 4 and the distal/second controllably expandable element 6. The device may be driven by an articulation movement powered by hydraulic actuated flexible cylinders and/or rods 59a, 59b, and 59c to bend the tip of the device. For example, the three hydraulically powered flex rods may enable the instrument to bend when individually extended/retracted with non-compressible fluid. As shown in the figure, flex rod 59a may contract, thereby pulling the tip of the device to the left. Alternatively, or at the same time, flex rod 59b and/or 59c may extend, thereby pushing the tip of the device to the left. The tip of the device may comprise working channel opening 62 and/or camera 63. During the movement, flex frame 60 allows for tight bend radius. FIG. 25B shows the one or more hydraulic actuator 57 (connected to power via one or more hydraulic lines 61) may comprise a soft cylinder. In addition, it may be connected to a flex rod 64 which is in turn connected an adjustable balloon assembly 65. Thus, flex rod 64 may contract, thereby pulling the proximal/first controllably expandable element 4 toward the tip of the device and closer to the distal/second controllably expandable element 6. FIG. 25C shows the one or more hydraulic actuator 57 (connected to power via one or more hydraulic lines 61) may comprise a hydraulic piston. Flex rods 59a, 59b, and 59c are connected to the hydraulic piston, which may pull and/or push the proximal/first controllably expandable element 4 and/or the distal/second controllably expandable element 6.

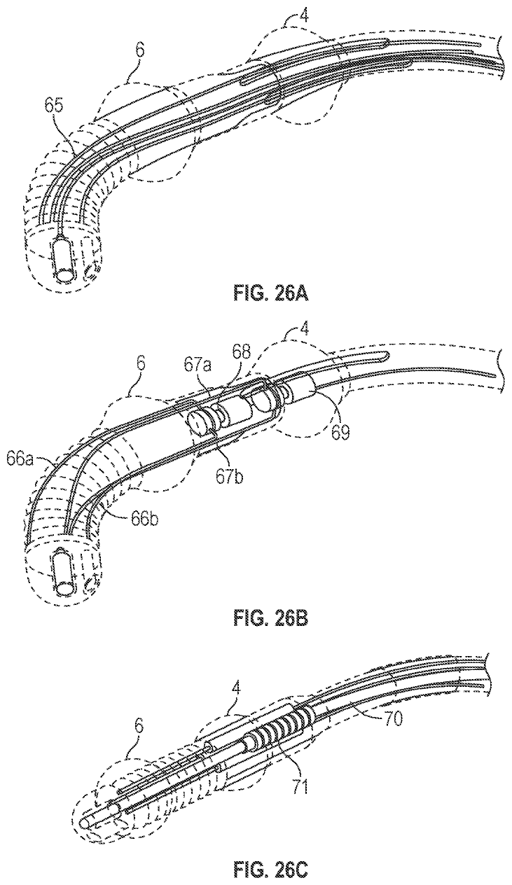

In any of the preceding embodiments, the device may comprises one or more of the following mechanisms: a cable articulation and/or propulsion mechanism, a motor/pulley articulation mechanism, and a linear servo motor propulsion mechanism. For example, as shown in FIG. 26A, a plurality of cables 65 are connected to an actuator to control the sliding movement between the outer member and the inner member of the device, and/or to control the articulation of the tip of the device. The device may comprise cables 65 running longitudinally through the device, the distal ends of said cables fixed in the tip of the device. The cables may be coupled with a plurality of motor-pulley systems, and individual pulling and pushing of said cables by the motor-pulley systems effects the bending of the tip of the device. As shown in FIG. 26B, the device may comprise a plurality of closed loop cables 66a/66b and 67a/67b running longitudinally through the device, the distal ends of said cables fixed in the tip of the device. The cables are coupled with a plurality of motor-pulley systems 68 and 69, respectively, and individual pulling and pushing of said cables by the motor-pulley systems effects the bending of the tip of the device. For example, closed loop cables 66a/66b are connected to motor-pulley system 68 on one end and the tip of the device on the other end, forming a closed loop. Similarly, closed loop cables 67a/67b are connected to motor-pulley system 69 on one end and the tip of the device on the other end, forming another closed loop. A flexible housing unit may surround the cable assembly to contain the articulation mechanism. The device may comprises a three phase servo motor actuator comprising a guidewire 70. As shown in FIG. 26C, linearly oriented coils 71 are energized in sequence to propel the balloon mechanism (e.g., a balloon anchor), such as balloons 4 and/or 6, forward and backward. The coils may be configured to slidably move along the guidewire. The device further comprises a bidirectional magnet installed on the balloon mechanism in order to integrate with the magnetic linear actuator.

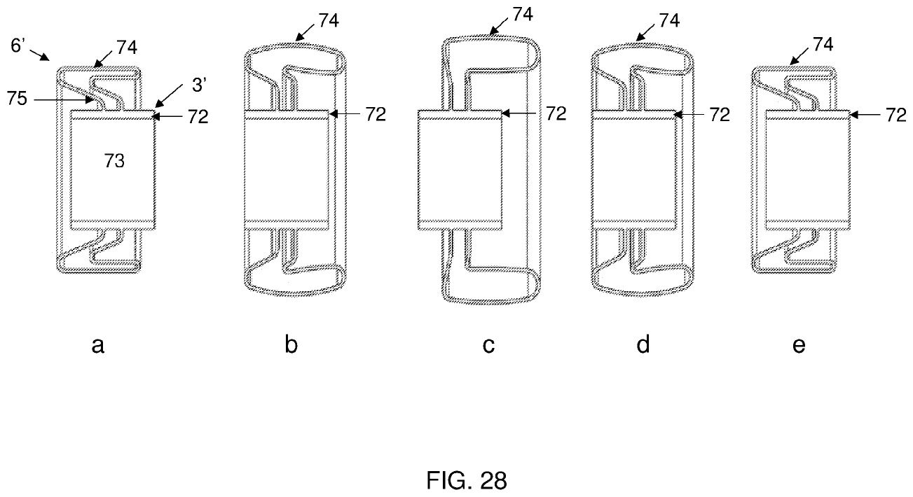

FIGS. 27-28 show an exemplary device disclosed herein configured to move within a body cavity. The device comprises a support (e.g., tubular structure such as tether) 3′ comprising a tubular wall 72 and a central lumen 73. Positioned along the length of the support (e.g., tubular structure such as tether) and in fluid communication with the central lumen are at least two controllably expandable elements, including a distal controllably expandable element 6′ (FIG. 28) and a proximal controllably expandable element 4′ (FIG. 27). The distal controllably expandable element and/or proximal controllably expandable element can comprise a flexible elastomeric hollow double walled part that has an outer surface 74 (e.g., an outside radial surface such as a traction surface). In some aspects, one of the double walls of the double walled part contacts the body lumen via the outside radial surface 74, while the other of the double walls is on the inside and does not contact the body lumen, like a donut or inflated tire. In some aspect, the outer surface 74 is configured to frictionally engage a wall of a body cavity or lumen. In some aspects, the device further comprises a propelling element 75 connecting the distal or proximal controllably expandable element to the tubular wall 72.

In some aspect, each of the distal and proximal controllably expandable elements is configured to expand radially outwardly. In FIG. 27a, for example, the proximal controllably expandable element 4′ is deflated (or not fully inflated). In FIG. 27b, the elastomeric hollow double walled part expands radially outwardly, and becomes frictionally engaged to a wall of a body cavity or lumen. As the controllably expandable element gets inflated in FIG. 27b, the propelling element 75 effects relative movement between the outer surface 74 and the support (e.g., tubular structure such as tether) 3′, e.g., while the outer surface is frictionally engaged to the body cavity wall, the tubular wall 72 (and hence the support (e.g., tubular structure such as tether) 3′) is moved in the distal direction, thereby advancing distally through the body cavity. FIG. 27c shows the controllably expandable element becomes even more inflated than in FIG. 27b, and the propelling element 75 effects further relative movement between the outer surface 74 and the support (e.g., tubular structure such as tether) 3′, e.g., moving the tubular wall 72 (and hence the support (e.g., tubular structure such as tether) 3′) further in the distal direction, thereby further advancing the support (e.g., tubular structure such as tether) (and the distal controllably expandable element 6′ which is in deflated or less inflated state) distally through the body cavity. In FIG. 27d and FIG. 27e, the proximal controllably expandable element 4′ is deflated, thereby allowing the support (e.g., tubular structure such as tether) 3′ to advance distally due to the expansion of the distal controllably expandable element 6′ and the propelling element thereon effecting movement of the support (e.g., tubular structure such as tether) 3′ in the distal direction. FIGS. 28a-28e show that the distal controllably expandable element 6′ can be similarly expanded or contracted (e.g., via inflation or deflation) to effect distal movement of the tubular wall 72 (and hence the support (e.g., tubular structure such as tether) 3′) while the outer surface 74 engages the body cavity wall, and consequently distal movement of the deflated or less inflated proximal controllably expandable element 4′ which is positioned on the support (e.g., tubular structure such as tether).

While FIGS. 27-28 shows the exemplary device provides both traction and motor functions using the controllably expandable elements (these elements are thus traction-motion elements such as traction-motion balloons) independent of a separate actuator such as a bellow motor and/or a balloon motor disclosed herein, it is within the present disclosure that an actuator disclosed herein or known in the art may be used in addition to one or more traction-motion element. In some embodiments, the actuator provides a motion mechanism in addition to one or more traction-motion element, thereby providing more flexibility and wider range of possible movement of the device within the body cavity.

In other embodiments, one or more traction-motion element can be used in any of the device disclosed herein, including the embodiments described in FIGS. 1-26. For example, the proximal/first controllably expandable element (e.g., element 4 on the outer tube 3, such as shown in FIG. 1) may be a traction-motion element disclosed herein, such as element 4′ shown in FIGS. 27-32. In other examples, the distal/second controllably expandable element (e.g., element 6 on the inner tube 5, such as shown in FIG. 1) may be a traction-motion element disclosed herein, such as element 6′ shown in FIGS. 27-32. In yet other examples, both the proximal/first controllably expandable element (e.g., element 4 on the outer tube 3, such as shown in FIG. 1) and the distal/second controllably expandable element (e.g., element 6 on the inner tube 5, such as shown in FIG. 1) may be a traction-motion element disclosed herein, such as element 4′ or 6′ shown in FIGS. 27-32. The traction-motion element(s) may provide an actuating/motion mechanism in addition to what is in certain embodiments herein, including those shown in FIGS. 1-26 and described in connection therewith.

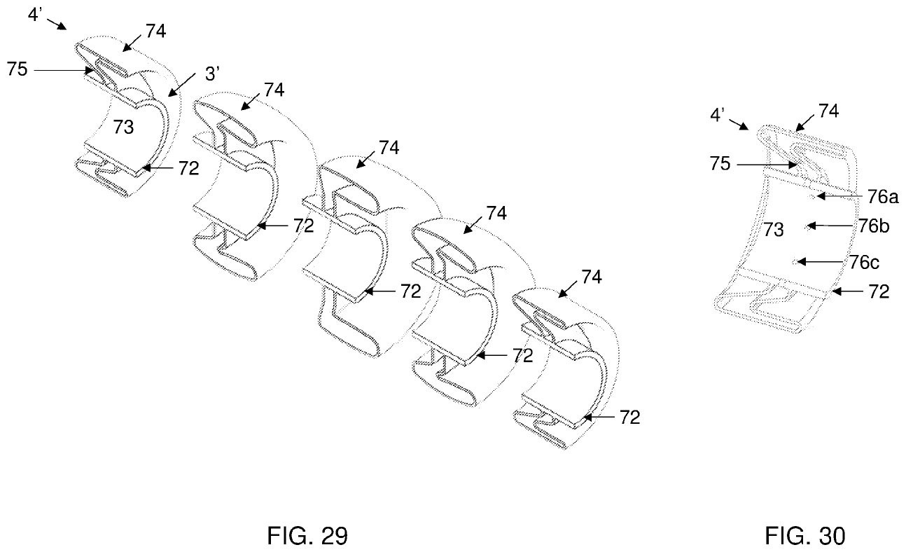

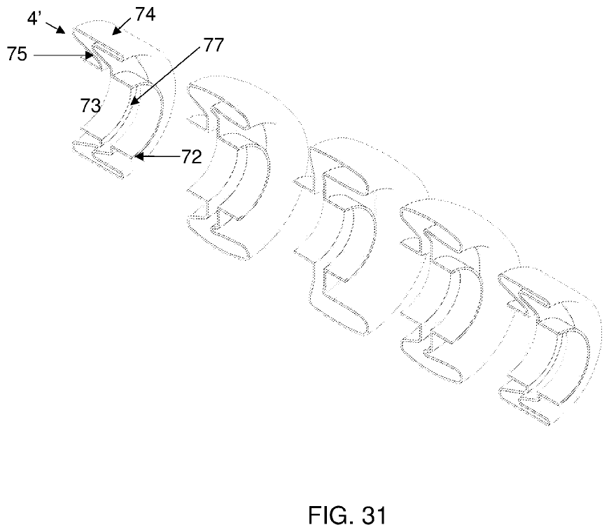

FIG. 29 shows crosssections of an exemplary device as a controllably expandable element of the device expands and contracts. In some embodiments, controllably expandable element 4′ (and/or 6′ which is not shown) is not in fluid communication with the central lumen 73. For example, expansion and/or contraction of the controllably expandable element may be controlled via a channel separate from the central lumen. In some embodiments, controllably expandable element 4′ (and/or 6′ which is not shown) is in fluid communication with the central lumen 73, and expansion and/or contraction of the controllably expandable element is controlled using fluid (gas and/or liquid) in the central lumen. For example, as shown in FIG. 30, on the tubular wall 72 there may be provided one or more aperture 76a, 76b, and 76c that connects the inside cavity of the controllably expandable element with the central lumen of the device. Another example shown in FIG. 31 utilizes a slit 77 to connect the inside cavity of the controllably expandable element with the central lumen. The slit may be partial or may form an entire circle.

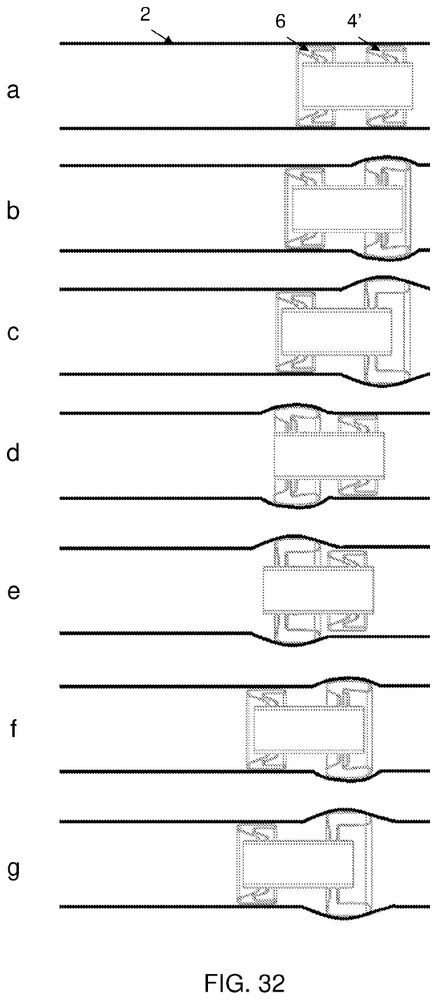

FIG. 32 shows an example where the controllably expandable elements 4′ and 6′ work in a controlled and coordinated fashion to provide both traction and motor functions, optionally independent of a separate motor or actuator. For instance, in FIG. 32a, the support (e.g., tubular structure such as tether) is placed inside a body cavity and both controllably expandable elements are deflated or not inflated, e.g., they do not frictionally engage a wall 2 of the body cavity such as the small intestine. In FIG. 32a, the proximal controllably expandable element 4′ becomes inflated to frictionally engage wall 2 while the distal controllably expandable element 6′ remains deflated and free to move inside the body cavity. This way, the propelling element of the proximal controllably expandable element 4′ advances the support (e.g., tubular structure such as tether) 3′ and element 6′ provided thereon in the distal direction. FIG. 32c shows element 4′ becomes even more inflated (while remaining frictionally engaged to wall 2), and propelling element of element 4′ moves support (e.g., tubular structure such as tether) 3′ and element 6′ thereon even more distally. In FIG. 32d, element 6′, now at a more distal position within the body cavity, is expanded and becomes frictionally engaged to wall 2, while element 4′ is deflated or becomes less inflated such that it becomes less frictionally engaged to wall 2 (e.g., element 4′ becomes free to move within the body lumen longitudinally). As such, expansion of element 6′ effects (through the propelling element of element 6′) distal movement of support (e.g., tubular structure such as tether) 3′ and element 4′ thereon. In FIG. 32e, element 6′ becomes even more expanded, and the propelling element of element 6′ drives even more distal movement of support (e.g., tubular structure such as tether) 3′ and element 4′ thereon. In FIG. 32f, element 4′, now at a more distal position within the body cavity, is expanded and becomes frictionally engaged to wall 2, while element 6′ is deflated or becomes less inflated such that it becomes less frictionally engaged to wall 2 (e.g., element 6′ becomes free to move within the body lumen longitudinally). As such, expansion of element 4′ effects (through the propelling element of element 4′) distal movement of support (e.g., tubular structure such as tether) 3′ and element 6′ thereon. FIG. 32g shows element 4′ becomes even more expanded and its propelling element drives even more distal movement of support (e.g., tubular structure such as tether) 3′ and element 6′ thereon. The above steps may be repeated to advance the support (e.g., tubular structure such as tether) and the device along the body cavity, such as small intestine, in order to reach portions of the cavity deeper in the patient body. Again, an actuator or motor disclosed herein or known in the art may be used in addition to one or more traction-motion element (e.g., 4′ or 6′) to control movement of the support (e.g., tubular structure such as tether), in the distal or proximal direction.

In any of the examples disclosed herein, a plurality of controllably expandable elements (e.g., traction-motion balloons) may be provided on the support (e.g., tubular structure such as tether). For instance, a plurality of traction-motion balloons can be in tandem to achieve an inchworm movement. In some examples, the distal and/or controllably expandable elements, e.g., as shown in FIG. 32, may each comprises a plurality of controllably expandable elements such as traction-motion balloons. For example, at least two, three, four, five, six, seven, eight, nine, or 10, or more than 10 traction-motion balloons can be stacked up next to each other, where the inflation progresses from one end to the other, pushing the support (e.g., tubular structure such as tether) in the center forward by virtue of the inflation of each of the traction-motion balloons. Each segment (e.g., a traction-motion balloon of a plurality of traction-motion balloons) may yield an incremental distance traveled. The same situation can be used to reverse the direction if the inflation travels the opposite way. In some embodiments, no motor balloon or motor bellows is necessary, and device achieves directional motion using the sequential inflation/deflation of the plurality of controllably expandable elements.

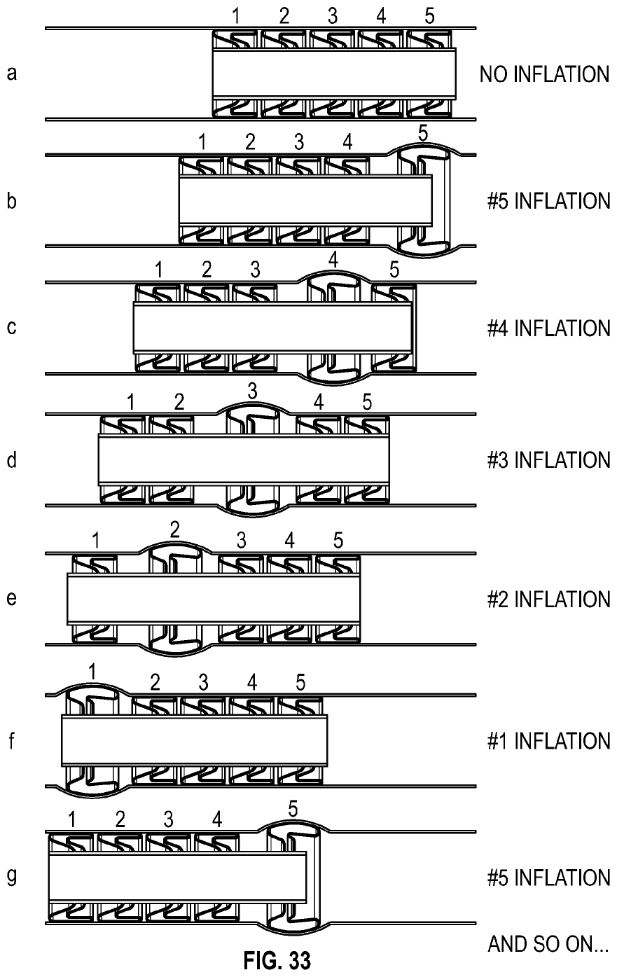

In some examples, as shown in FIG. 33a, a plurality of traction-motion balloons 1-5 are provided on the central support (e.g., tubular structure such as tether) and are deflated or not inflated, e.g., they do not frictionally engage a wall of the body cavity such as the small intestine. In FIG. 33b, traction-motion balloon 5 is inflated to frictionally engage the body cavity wall while traction-motion balloons 1-4 remain deflated (or not as inflated as traction-motion balloon 5) and free to move inside the body cavity. The inflation of traction-motion balloon 5 drives the central support (e.g., tubular structure such as tether) forward (compare the position of the central support (e.g., tubular structure such as tether) in FIG. 33b to that in FIG. 33a). Next, traction-motion balloon 5 is deflated, traction-motion balloon 4 is inflated to frictionally engage the body cavity wall while traction-motion balloons 1-3 remain deflated. The inflation of traction-motion balloon 4 drives the central support (e.g., tubular structure such as tether) forward further down the body cavity, while traction-motion balloons 1-3 and 5 are free to move inside the body cavity as shown in FIG. 33c. As shown in FIGS. 33d-f, traction-motion balloon 3, traction-motion balloon 2, and traction-motion balloon 1 are sequentially inflated (while the rest of the traction-motion balloon can move along with the central support (e.g., tubular structure such as tether)), each providing a further movement of the central support (e.g., tubular structure such as tether). In some aspects, a device comprising segmented element soft robot, when activated sequentially, propels itself forward through a lumen in the anatomy. In any of the embodiments disclosed herein, the device is also capable of reverse motion when the sequential inflation is done in the opposite direction, for example, because of the recoil or hysteresis of the material used to create each segment.

In any of the examples disclosed herein, the outer surface may be a wavy, ribbed, and/or saw tooth shaped or patterned surface configured to frictionally engage the body cavity wall. In some embodiments, when the traction-motion element is deflated, the wavy, ribbed, and/or saw tooth shapes or patterns on the outer surface shrink down, effectively folding up.

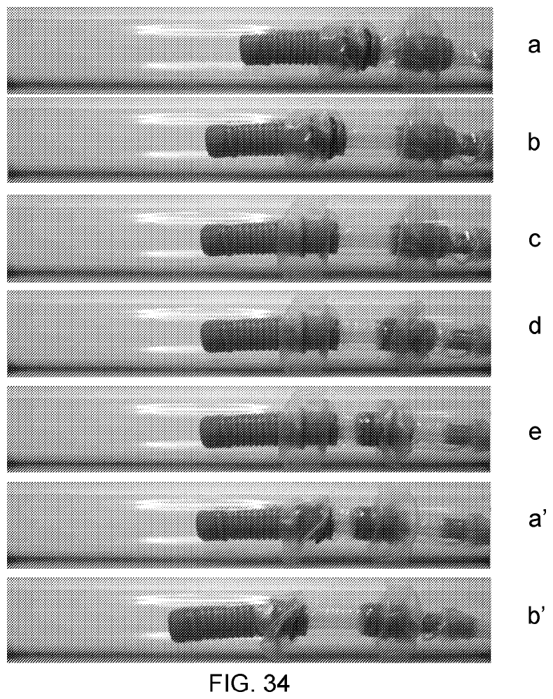

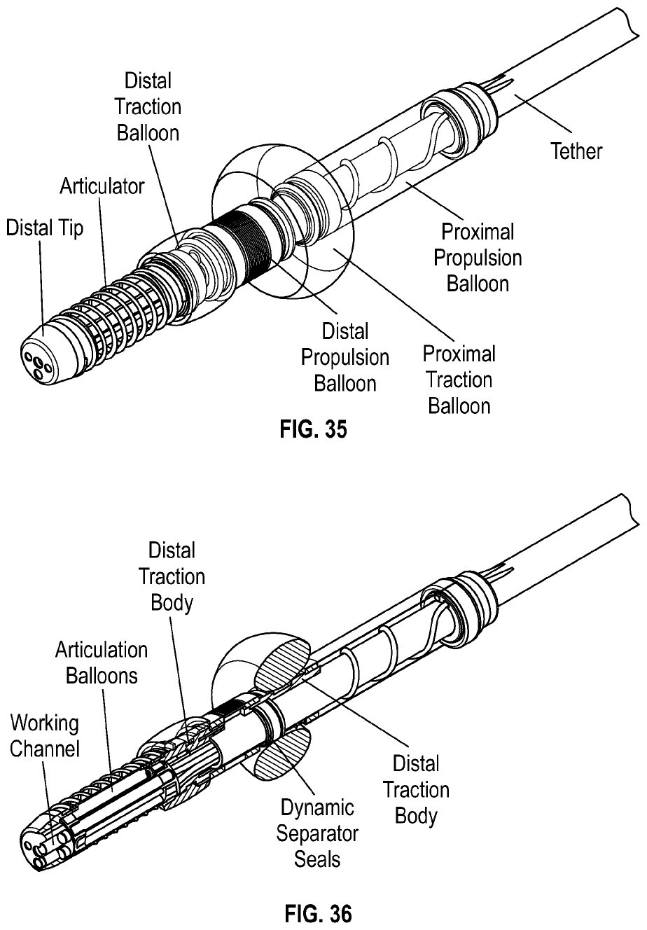

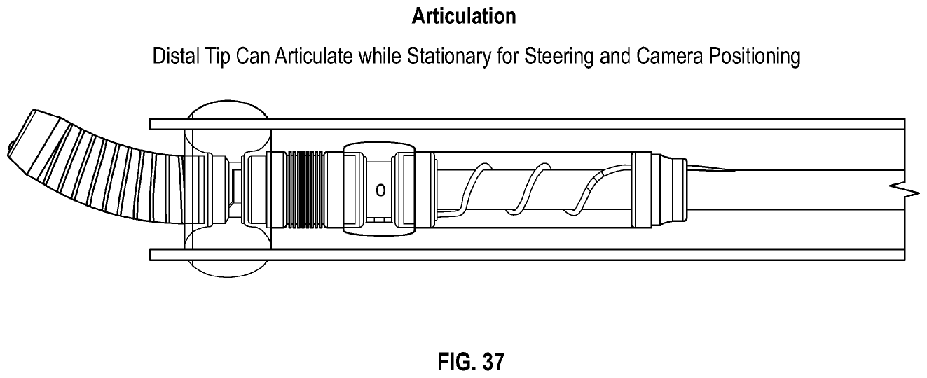

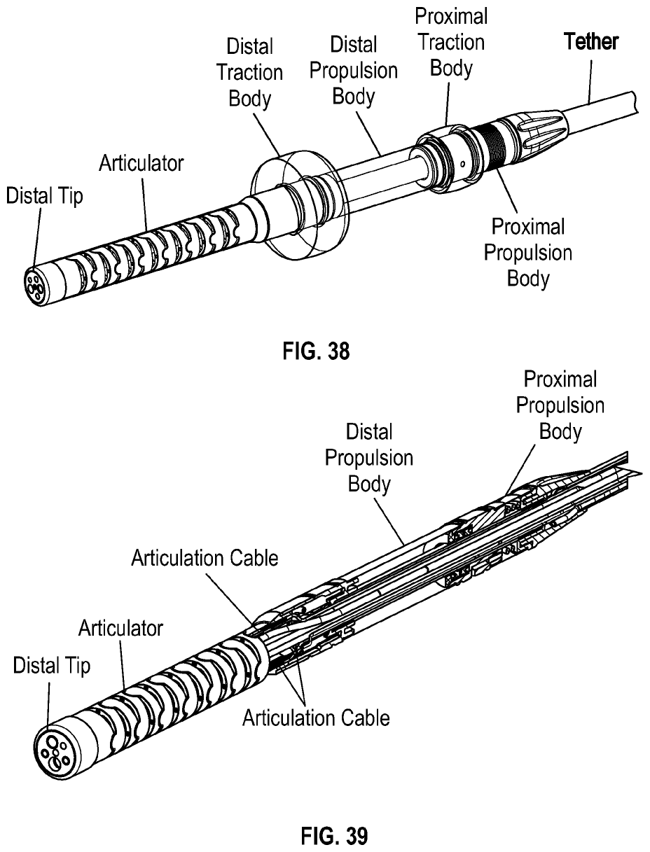

In some embodiments, disclosed herein is a method in which the controllably expandable elements of a device work in a controlled and coordinated fashion to drive the device within the body cavity. For example, FIG. 34 shows in step a, the proximal/first radially expandable element (e.g., the traction balloon on the right) is inflated and engages the body cavity wall, while the distal/second radially expandable element (e.g., the traction balloon on the left) is deflated and does not engage the body cavity wall. In step b, positive pressure is provided to expand the distal/second longitudinally expandable element (e.g., the propulsion balloon on the left), which may help collapse the proximal/first longitudinally expandable element (e.g., the propulsion balloon on the right) while moving the support (e.g., tubular structure such as tether) (including the tip of the device and the deflated distal/second radially expandable element) distally. In step c, when the deflated distal/second radially expandable element reaches a more distal position, it is inflated to engage the body cavity wall, and the proximal/first radially expandable element is deflated. In step d, positive pressure is provided to expand the proximal/first longitudinally expandable element, which expansion may help collapse the distal/second longitudinally expandable element while moving the deflated proximal/first radially expandable element (which has a floating seal) distally and closer to the inflated distal/second radially expandable element. In step e, the distal/second radially expandable element is deflated and the proximal/first radially expandable element, once reaching a more distal position, is inflated to engage the body cavity wall. Step a′ is similar to step a and the process can be repeated in step b′ (similar to step b) and subsequent steps (not shown). FIG. 34 shows the expansion of the proximal longitudinally expandable element and the collapsing of the distal longitudinally expandable element can effect sliding movement of the proximal radially expandable element along the length of the elongated support, and the collapsing of the proximal longitudinally expandable element and the expansion of the distal longitudinally expandable element can effect movement of the distal radially expandable element.

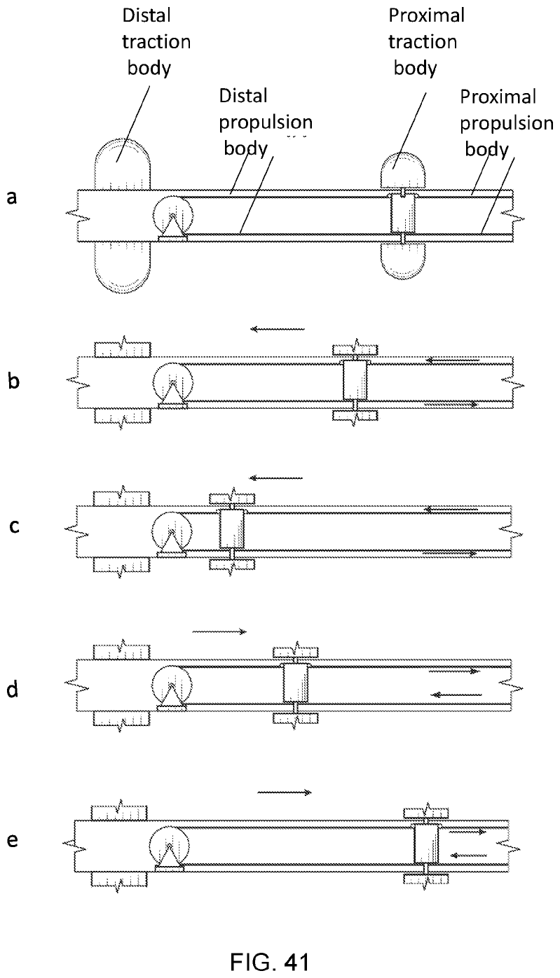

In some embodiments, disclosed herein is a device and a method in which the controllably expandable elements of the device work in a controlled and coordinated fashion to drive the device within the body cavity. For example, panel a of FIG. 41 shows an exemplary device comprising a closed loop cable connected with a pulley system, wherein the distal end of the closed loop cable is between the proximal and the distal radially expandable element (e.g., traction balloons), wherein the proximal radially expandable element (e.g., the traction balloon on the right labeled “proximal traction body”) is connected to the cable in the pulley system (e.g., the traction balloon is fixed onto the upper segment of the cable), thus dividing the pulley system into a proximal part and a distal part (e.g., the right and the left segments of the pulley system labeled proximal and distal propulsion bodies, respectively). In panel b, the distal radially expandable element (e.g., the left traction balloon) is expanded to engage the wall of a body cavity, and the proximal radially expandable element is collapsed (not shown). The proximal part of the pulley system (e.g., the right segment of the closed loop cable) is expanded by movement of the cable and the distal part of the pulley system (e.g., the left segment of the closed loop cable) is simultaneously collapsed, thereby pushing the proximal radially expandable element (e.g., the right balloon) forward. In panel c, the proximal radially expandable element is pushed forward, and expanded radially outwardly to engage a wall of the tube. In panels d and e, the proximal radially expandable element may be expanded to engage the body cavity wall (e.g., the right traction balloon is inflated, not shown) while the distal radially expandable element may be collapsed (e.g., the left traction balloon is deflated, not shown). The proximal part of the pulley system (e.g., the right segment of the closed loop cable) is collapsed by movement of the cable and the distal part of the pulley system (e.g., the left segment of the closed loop cable) is simultaneously expanded, thereby pushing the distal radially expandable element (e.g., the left balloon) forward. Steps in panels b-e may be repeated, thereby propel the distal end of the device further forward. Panels d and e also show a method for reverse locomotion, wherein the proximal radially expandable element is retracted (e.g., deflated, not shown), the distal radially expandable element is expanded (e.g., inflated, not shown), the distal part of the pulley system (e.g., the left segment of the closed loop cable) is expanded, thereby bringing the proximal radially expandable element backward in the more proximal direction.

In some embodiments, there is a need for a device comprising a flexible region, e.g., between the radially expandable elements, that can transmit force to achieve bi-directional movement. In some embodiments, the flexible region comprises one or more structures (e.g., layers) can be act as one piece in unity or independently if needed. In some embodiments, the flexible region comprises a core structure or layer, surround by a middle structure (e.g., cable) or layer, which is in turn surrounded by an outer structure (e.g., cable) or layer. In some embodiments, the core structure or layer is flexible and of sufficient tension, while the middle and/or outer structures or layers are cable of being compressed. In some embodiments, a device disclosed herein comprises one or more cables can be act as one piece in unity or independently if needed. In some embodiments, the one or more cables are configured to pull and/or push in order to compress or decompress the middle and/or outer structures or layers. In some embodiments, the middle structure comprises one or more cables (e.g., compressive, load carrying cables). In some embodiments, the middle structure comprises one or more cables that are configured to push and/or pull, and the outer structure comprises one or more springs. In some embodiments, the middle structure comprises one or more cables that are configured to push and/or pull, and the outer structure comprises another one or more cables that are configured to push and/or pull.

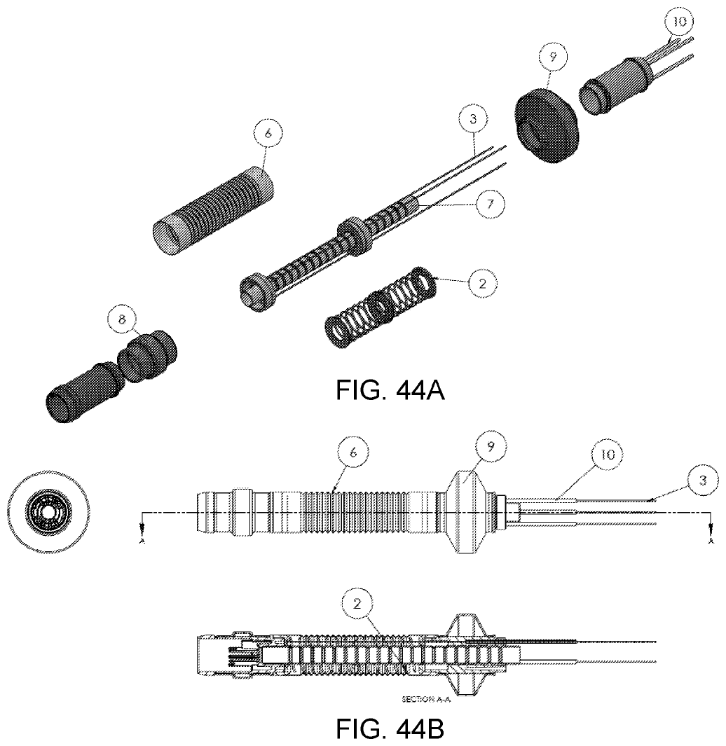

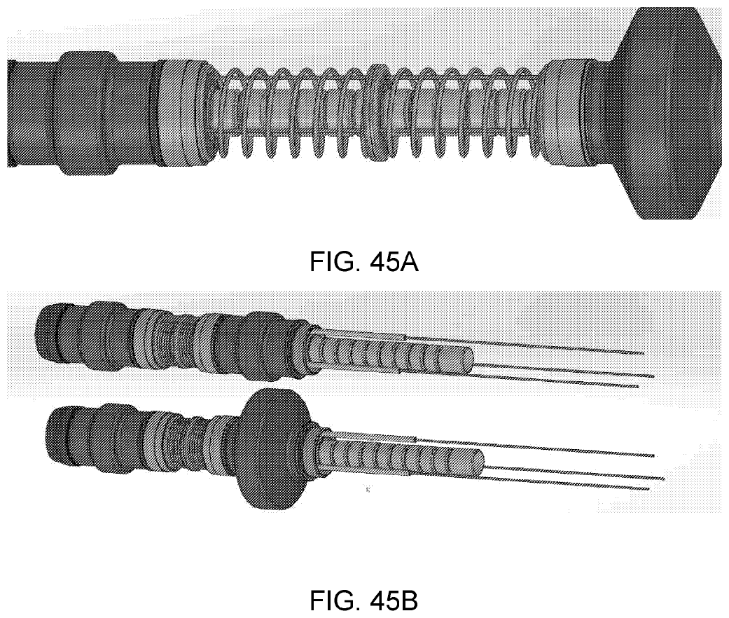

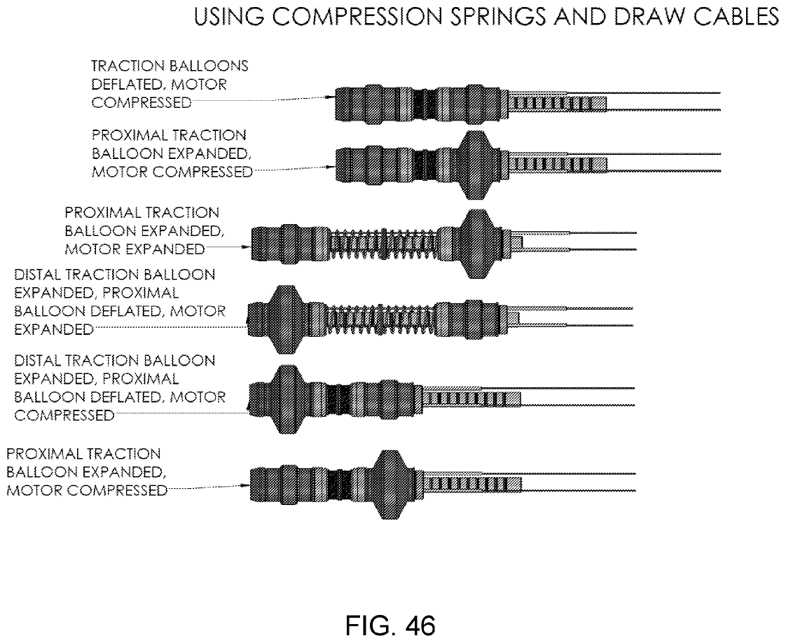

FIG. 44A shows an exemplary device comprising one or more compression springs and one or more cables (e.g., draw cables) for pushing and/or pulling one or more radially expandable structures. The device comprises pull collar 1 (e.g., for compression) which can be attached to the distal traction balloon 8. The device further comprises compression spring 2 between pull collar 1 and a collar attached to the proximal traction balloon 9. The compression spring 2 can be one spring or comprise two or more springs connected via a separator between one another. The separator can act as a cable guide and may comprise one or more through holes to accommodate the push/pull cable(s) 3, which can function as pull strings for compression. In some examples, the device comprises at least two push/pull cables and the separator comprises a corresponding number of through holes to accommodate the push/pull cables. In some examples, the device comprises at least three push/pull cables and the separator comprises a corresponding number of through holes to accommodate the push/pull cables. In some examples, the push/pull cables 3 are attached to pull collar 1, and pass through one or more separators between springs, through the collar attached to the proximal traction balloon, and/or through the proximal traction balloon. In some examples, the push/pull cables 3 can be in an outer sheath 10, which can be provided proximal to the proximal traction balloon 9. The device can comprise a containment membrane 6 surrounding the compression spring 2 and the push/pull cable(s) 3, which in turn may surround a flexible inner core 7, which may be cannulated to increase flexibility (e.g., to facilitate bending while the device navigates in a body cavity such as the small intestine). FIG. 44B shows a side view and a cross section of the device. FIG. 45A shows the device can comprise a flexible region between two radially expandable structures, where the proximal radially expandable structure is expanded and the distal radially expandable structure is not. The flexible region may comprise a plurality of compression springs. A separator between two compression springs may act as a cable guide. FIG. 45B shows the compression springs are in a compressed configuration, when the proximal radially expandable structure is collapsed (upper panel) or expanded (lower panel). The flexible region can function as a locomotion system (e.g., comprising compression springs) which may be controlled by one or more motors (e.g., step motors, for pulling and/or pushing one or more cables), e.g., through the push/pull cables (e.g., draw cables). FIG. 46 shows an exemplary method of using the device comprising a flexible region to effect movement of the device inside a body cavity. In some embodiments, the device can achieve a stroke of about 25 mm, about 50 mm, about 75 mm, or about 100 mm, or even longer, for instance, with two or more separators. In some embodiments, the cables can comprise bicycle brake cable type of cables (e.g., Bowden cables). In some embodiments, the cables are rigidly attached to the distal collar (e.g., pull collar 1). In some embodiments, the locomotion system comprising a flexible region can be used to provide sufficient force (e.g., impact drive) to enable the device to cross a transition zone inside the body cavity. For instance, the force generated using the flexible region can push the distal tip and/or the distal traction balloon through the ileocecal valve, a sphincter muscle situated at the junction of the ileum (last portion of the small intestine) and the colon (first portion of the large intestine), for ileocaecal junction (ICJ) crossing. In some embodiments, one or more of the push/pull cables can be selectively adjusted to bend the flexible core to provide “rear wheel steering”—that is, steering by adjusting the position and/or configuration of a proximal part of the device rather than the distal end of the device. In some embodiments, the flexible region comprising springs and cables can be configured to provide rear end wiggle ability such that the a proximal region of the device can wiggle or otherwise change configuration, e.g., to facilitate passage of the proximal balloon through the ICJ. By using the flexible region comprising springs and cables instead of balloons (e.g., drive balloons) the diameter of the device can be decreased. In some embodiments, the flexible region can be configured to provide a force sufficient to drive locomotion, e.g., a force of at least about 0.5 Lbs, 1 Lbs, 1.5 Lbs, 2.0 Lbs, 2.5 Lbs, 3.0 Lbs, or more. In some embodiments, the flexible region can be configured to provide incremental movement and/or precision control of the movement of the device inside a body cavity. In some embodiments, the flexible region can be configured to permit electronic algorithm control.

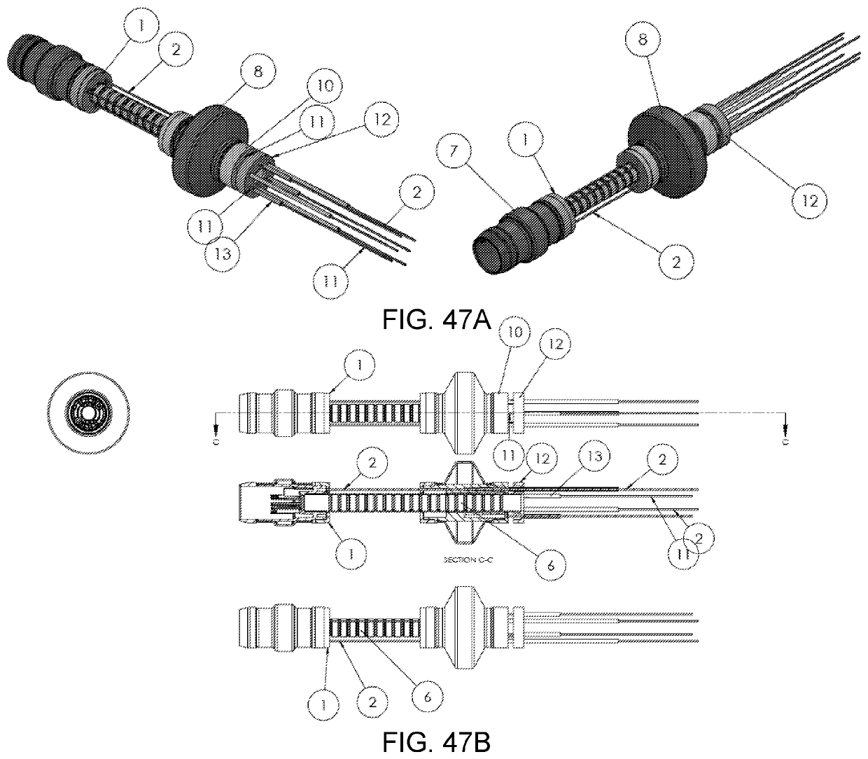

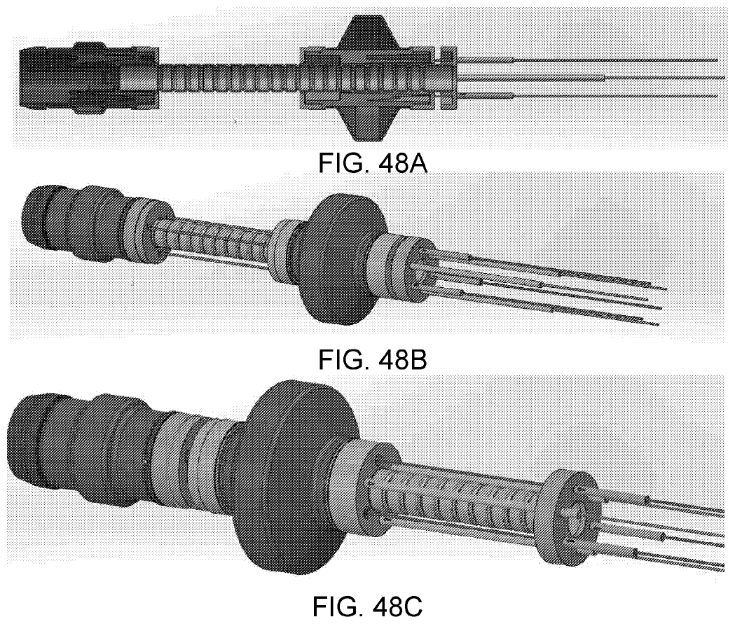

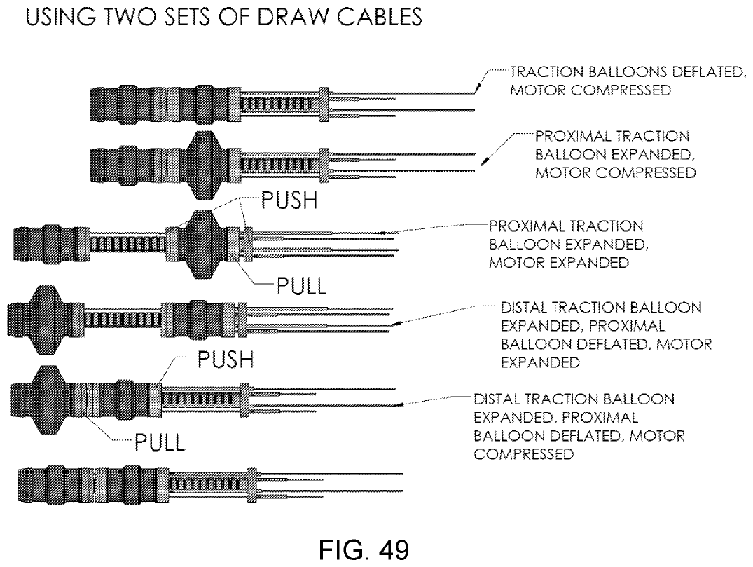

FIG. 47A shows an exemplary device comprising one or more cables (e.g., draw cables), and in particular, two or more sets of cables, each set for pushing and/or pulling a radially expandable structure. The device comprises pull collar 1 (e.g., for compression) which can be attached to the distal traction balloon 7. The device does not need to comprise a compression spring, but may comprise push collar 12 (e.g., for distraction) attached to the proximal traction balloon 8. In some examples, the push/pull cables (e.g., pull string for compression) 2 are attached to pull collar 1, and pass through a distal collar attached to the proximal traction balloon 8, through the proximal traction balloon 8, and/or a proximal collar (e.g., push/pull collar) 10 attached to the proximal traction balloon 8. In some examples, the device further comprises push/pull cables (e.g., pull string for distraction) 11 attached to push collar 12 (e.g., for distraction), which can move relative to push/pull collar 10 and proximal traction balloon 8. In some examples, the push/pull cables (e.g., pull string for compression) 2 can pass through push collar 12. In some examples, the cables 2 and 11 can each be in an outer sheath. For example, cables 2 can be in pull cable outer sheath 9 for compression and cables 11 can be in push cable outer sheath 13 for distraction. The outer sheaths can be provided proximal to the proximal traction balloon 8, proximal to proximal collar (e.g., push/pull collar) 10, and/or proximal to push collar 12. The device can comprise a containment membrane 5 surrounding the cable which in turn may surround a flexible inner core 6, which may be cannulated to increase flexibility (e.g., to facilitate bending while the device navigates in a body cavity such as the small intestine). FIG. 47B shows side views and a cross section of the device. FIG. 48A shows a cross section of the device comprising a flexible region between two radially expandable structures, where the proximal radially expandable structure is expanded and the distal radially expandable structure is not. The flexible region may comprise different sets of cables. One or more collars may be provided (in addition to the pull collar and/or the push collar) to act as a cable guide. FIG. 48B shows a side view of the device. FIG. 48C shows the device in a compressed configuration, when the proximal radially expandable structure is expanded. The flexible region can function as a locomotion system (e.g., comprising different sets of push and/or pull cables) which may be controlled by one or more motors (e.g., step motors, for pulling and/or pushing one or more cables), e.g., through the push/pull cables (e.g., draw cables). FIG. 49 shows an exemplary method of using the device comprising a flexible region to effect movement of the device inside a body cavity. In some embodiments, the device can achieve a stroke of about 25 mm, about 50 mm, about 75 mm, or about 100 mm, or even longer. In some embodiments, the cables can comprise bicycle brake cable type of cables (e.g., Bowden cables). In some embodiments, the cables are attached to a collar and/or pass through through holes of one or more other collars. In some embodiments, a set of cables are rigidly attached to a distal collar which may be used to pull the distal tractional balloon proximally. In some embodiments, another set of cables are rigidly attached a proximal collar which can be used to push and advance the distal tractional balloon. In some embodiments, the locomotion system comprising a flexible region comprising different sets of cables can be used to provide sufficient force (e.g., impact drive) to enable the device to cross a transition zone inside the body cavity. For instance, the force generated using the flexible region can push the distal tip and/or the distal traction balloon through the ileocecal valve for ileocaecal junction (ICJ) crossing. In some embodiments, one or more of the push/pull cables can be selectively adjusted to bend the flexible core to provide “rear wheel steering”—that is, steering by adjusting the position and/or configuration of a proximal part of the device rather than the distal end of the device. In some embodiments, the flexible region comprising different sets of cables, e.g., one set for pulling and/or pushing a distal traction balloon and another set for pulling and/or pushing a proximal traction balloon, can be configured to provide rear end wiggle ability such that the a proximal region of the device can wiggle or otherwise change configuration, e.g., to facilitate passage of the proximal balloon through the ICJ. By using the flexible region comprising cables instead of balloons (e.g., drive balloons) the diameter of the device can be decreased. In some embodiments, the flexible region can be configured to provide a force sufficient to drive locomotion, e.g., a force of at least about 0.5 Lbs, 1 Lbs, 1.5 Lbs, 2.0 Lbs, 2.5 Lbs, 3.0 Lbs, or more. In some embodiments, the flexible region can be configured to provide incremental movement and/or precision control of the movement of the device inside a body cavity. In some embodiments, the flexible region can be configured to permit electronic algorithm control.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The term “about” as used herein refers to the usual error range for the respective value readily known. Reference to “about” a value or parameter herein includes (and describes) embodiments that are directed to that value or parameter per se. For example, description referring to “about X” includes description of “X”.

As used herein, a “subject” is a mammal, such as a human or other animal, and typically is human.

### EXEMPLARY EMBODIMENTS

Among the provided embodiments are but not limited to:

Embodiment 1. A device configured to move within a body cavity, the device comprising: a) an elongated support; b) a proximal radially expandable element and a distal radially expandable element positioned along the length of the elongated support, optionally wherein the radially expandable elements are independently controllably expandable, wherein the radially expandable elements comprise outer surfaces configured to frictionally engage a wall of a body cavity, and wherein the distal radially expandable element is fixed relative to the elongated support; and c) a locomotion system comprising: i) a proximal locomotion element having a part that is fixed relative to the proximal radially expandable element and slidable along the length of the elongated support, such that the proximal radially expandable element is slidable along the length of the elongated support, and ii) a distal locomotion element having a part fixed relative to the elongated support, wherein the locomotion system effects sliding movement of the proximal radially expandable element along the length of the elongated support, thereby effecting relative movement between the radially expandable elements and movement of the device within the body cavity.

Embodiment 2. The device of embodiment 1, wherein the elongated support comprises a tubular wall and a lumen, optionally wherein the lumen is a central lumen.

Embodiment 3. The device of embodiment 2, wherein one or both of the expandable elements and/or one or both of the locomotion elements are in fluid or gas communication with one or more chambers, one or more channels, one or more tubes, and/or one or more wires in the central lumen.

Embodiment 4. The device of any one of embodiments 1-3, wherein any one or more of the expandable elements and locomotion elements are independently controlled.

Embodiment 5. The device of any one of embodiments 1-4, wherein the locomotion elements are configured to expand or collapse along the length of the elongated support, optionally wherein the locomotion elements are configured to expand or collapse only along the length of the elongated support and/or are not radially expandable.

Embodiment 6. The device of any one of embodiments 1-5, wherein the proximal and the distal radially expandable elements are capable of expanding radially outwardly to engage a wall of a body cavity, optionally wherein friction augmenting features are molded into the proximal and/or distal radially expanding elements.

Embodiment 7. The device of any one of embodiments 1-6, wherein alternating extensions and retractions of a distance between the outer surfaces of the proximal and the distal radially expandable elements effects movement of the device within the body cavity.

Embodiment 8. The device of any one of embodiments 1-7, wherein the elongated support further comprises one or more aperture on a distal end, and/or wherein the elongated support further comprises one or more medium channels separately connected to the radially expandable elements, optionally wherein the medium comprises gas, liquid or a mixture thereof, and optionally wherein the medium comprises a vapor, and/or wherein the elongated support further comprises one or more wires or channels for a camera or sensor, optionally wherein the sensor is a pressure sensor and/or an image sensor, and/or wherein the elongated support houses or engages an endoscope assembly.

Embodiment 9. The device of any one of embodiments 1-8, further comprising an articulation element capable of effecting articulation of a distal end of the device, optionally wherein the distal end of the device is the distal end of the elongated support or the distal end of the device directly or indirectly engages the distal end of the elongated support.

Embodiment 10. The device of embodiment 9, wherein the articulation element enables steering of the device, optionally wherein the device comprises a machine vision element that digitally recognizes structures assisting in navigation and identifying anomalies such as lesions and polyps, optionally wherein the machine vision assists in navigation and/or transmitting location of structures such as when moving from the large intestine to the small intestine.

Embodiment 11. The device of any one of embodiments 9-10, wherein the articulation element comprises a motor.

Embodiment 12. The device of any one of embodiments 9-11, wherein the articulation element comprises one or more closed loop cables configured to effect articulation.

Embodiment 13. The device of any one of embodiments 1-12, further comprising one or more channels not in connection with the expandable elements.

Embodiment 14. The device of any one of embodiments 1-13, wherein the proximal radially expandable element is a proximal balloon.

Embodiment 15. The device of any one of embodiments 1-14, wherein the distal radially expandable element is a distal balloon.

Embodiment 16. The device of any one of embodiments 1-15, wherein the proximal radially expandable element directly or indirectly engages one or more floating elements configured to slide along the length of the elongated support, thereby sliding the proximal radially expandable element along the length of the elongated support.

Embodiment 17. The device of any one of embodiments 1-16, wherein the locomotion system comprises two longitudinally expandable elements.

Embodiment 18. The device of embodiment 17, wherein the locomotion system comprises a proximal longitudinally expandable element and a distal longitudinally expandable element, optionally wherein the longitudinally expandable elements are independently controllably expandable, and optionally wherein the longitudinally expandable elements each comprises a structure independently selected from the group consisting of a compliant balloon, a semi-compliant balloon, a pressure balloon, and a bellow.

Embodiment 19. The device of any one of embodiments 1-16, wherein the locomotion system comprising a pulley system.

Embodiment 20. The device of embodiment 19, wherein the pulley system comprises a proximal floating element, a distal wheel, and a cable connected to the proximal floating element and engaging the distal wheel.

Embodiment 21. The device of any one of embodiments 1-20, wherein the distal locomotion element comprises a part fixed to the distal expandable element.

Embodiment 22. A device configured to move within a body cavity, the device comprising: a) an elongated support; b) a proximal radially expandable element and a distal radially expandable element positioned along the length of the elongated support, wherein the radially expandable elements comprise outer surfaces configured to frictionally engage a wall of a body cavity, and wherein the distal radially expandable element is fixed relative to the elongated support; c) a locomotion system comprising a proximal longitudinally expandable element and a distal longitudinally expandable element connected by a floating seal, wherein: i) the proximal longitudinally expandable element is proximal to the proximal radially expandable element, and the floating seal is fixed relative to the proximal radially expandable element and slidable along the length of the elongated support, such that the proximal radially expandable element is slidable along the length of the elongated support, and ii) the distal longitudinally expandable element is proximal to the distal radially expandable element, and the distal end of the distal longitudinally expandable element is fixed relative to the elongated support, wherein the locomotion system effects sliding movement of the proximal radially expandable element along the length of the elongated support, thereby effecting relative movement between the radially expandable elements and movement of the device within the body cavity.

Embodiment 23. The device of embodiment 22, wherein the proximal and the distal longitudinally expandable elements are configured to expand or collapse along the length of the elongated support, optionally wherein the proximal and the distal longitudinally expandable elements configured to expand or collapse only along the length of the elongated support and/or are not radially expandable.

Embodiment 24. The device of embodiments 22 or 23, wherein alternating expansion and collapsing of the proximal and the distal longitudinally expandable elements do not change the distance between the proximal end of the proximal longitudinally expandable element and the distal end of the distal longitudinally expandable element.

Embodiment 25. The device of any one of embodiments 22-24, wherein the distance between the proximal end of the proximal longitudinally expandable element and the distal end of the distal longitudinally expandable element is pre-determined.

Embodiment 26. The device of any one of embodiments 22-25, wherein expansion of the proximal and/or the distal longitudinally expandable elements is effected by positive pressure, optionally wherein negative pressure is proactively and alternatively applied to the longitudinally expandable elements in order to evacuate previously applied positive pressure, and optionally wherein the proximal and/or the distal longitudinally expandable elements do not passively deflate.

Embodiment 27. The device of any one of embodiments 22-26, wherein the expansion of the proximal longitudinally expandable element and the collapsing of the distal longitudinally expandable element effects sliding movement of the proximal radially expandable element along the length of the elongated support.

Embodiment 28. The device of any one of embodiments 22-27, wherein the collapsing of the proximal longitudinally expandable element and the expansion of the distal longitudinally expandable element effects movement of the distal radially expandable element.

Embodiment 29. The device of any one of embodiments 22-28, wherein the elongated support further comprises one or more aperture on a distal end, and/or wherein the elongated support further comprises one or more medium channels separately connected to the radially expandable elements, optionally wherein the medium comprises gas, liquid or a mixture thereof, and optionally wherein the medium comprises a vapor, and/or wherein the elongated support further comprises one or more wires or channels for a camera or sensor, optionally wherein the sensor is a pressure sensor and/or an image sensor, and/or wherein the elongated support houses or engages an endoscope assembly.

Embodiment 30. A method for locomotion of the device of any one of embodiments 22-29 through a body cavity, the method comprising: i. expanding the proximal radially expandable element radially outwardly to engage a wall of the body cavity, thereby fixing the proximal radially expandable element to a first position in the body cavity; ii. expanding (e.g., using positive pressure) the distal longitudinally expandable element along the elongated support to increase the distance between the proximal and the distal radially expandable elements; iii. expanding the distal radially expandable element radially outwardly to engage a wall of the body cavity; iv. retracting the proximal radially expandable element radially inwardly; v. retracting (e.g., using negative pressure) the distal longitudinally expandable element, and/or expanding (e.g., using positive pressure) the proximal longitudinally expandable element along the elongated support, thus effecting sliding movement of the proximal radially expandable element along the elongated support and decreasing the distance between the proximal and the distal radially expandable elements; and vi. optionally expanding the proximal radially expandable element radially outwardly to engage a wall of the body cavity, thereby fixing the proximal radially expandable element to a second position in the body cavity, optionally the second position is distal to the first position.

Embodiment 31. The method of embodiment 30, further comprising: vii. expanding (e.g., using positive pressure) the distal longitudinally expandable element along the elongated support to increase the distance between the proximal and the distal radially expandable elements.

Embodiment 32. A method for locomotion of the device of any one of embodiments 22-29 through a body cavity, the method comprising: i. expanding the distal radially expandable element radially outwardly to engage a wall of the body cavity, thereby fixing the distal radially expandable element to a first position in the body cavity; ii. expanding (e.g., using positive pressure) the proximal longitudinally expandable element along the elongated support, thus effecting sliding movement of the proximal radially expandable element along the elongated support and decreasing the distance between the proximal and the distal radially expandable elements; iii. expanding the proximal radially expandable element radially outwardly to engage a wall of the body cavity; iv. retracting the distal radially expandable element radially inwardly; v. retracting (e.g., using negative pressure) the proximal longitudinally expandable element, and/or expanding (e.g., using positive pressure) the distal longitudinally expandable element along the elongated support; and vi. optionally expanding the distal radially expandable element radially outwardly to engage a wall of the body cavity, thereby fixing the distal radially expandable element to a second position in the body cavity, optionally the second position is distal to the first position.

Embodiment 33. The method of embodiment 32, further comprising: vii. expanding (e.g., using positive pressure) the proximal longitudinally expandable element along the elongated support to decrease the distance between the proximal and the distal radially expandable elements

Embodiment 34. A device configured to move within a body cavity, the device comprising: a) an elongated support; b) a proximal radially expandable element and a distal radially expandable element positioned along the length of the elongated support, wherein the radially expandable elements comprise outer surfaces configured to frictionally engage a wall of a body cavity, and wherein the distal radially expandable element is fixed relative to the elongated support; c) a pulley system comprising: i) a proximal floating element that is fixed relative to the proximal radially expandable element and slidable along the length of the elongated support, such that the proximal radially expandable element is slidable along the length of the elongated support, ii) a distal wheel fixed relative to the elongated support, and iii) a cable connected to the proximal floating element and engaging the distal wheel, such that the cable is configured to pull the proximal floating element in the distal or proximal direction, wherein the pulley system effects sliding movement of the proximal radially expandable element along the length of the elongated support, thereby effecting relative movement between the radially expandable elements and movement of the device within the body cavity.

Embodiment 35. The device of embodiment 34, wherein the cable is a closed loop cable.

Embodiment 36. The device of any one of embodiments 34-35, wherein movement of the cable effects movement of the proximal radially expandable element along the elongated support, thereby effecting alternative extensions and retractions of a distance between the outer surfaces of the proximal and the distal radially expandable elements along the length of the elongated support.

Embodiment 37. The device of any one of embodiments 34-36, wherein the radially expandable elements are independently controllably expandably, and, optionally wherein the elongated support further comprises one or more medium channels separately connected to the radially expandable elements, optionally wherein the medium comprises gas, liquid or a mixture thereof and optionally wherein the medium comprises a vapor, and/or wherein the elongated support further comprises one or more wires or channels for a camera or sensor, optionally wherein the sensor is a pressure sensor and/or an image sensor, and/or wherein the elongated support houses or engages an endoscope assembly.

Embodiment 38. The device of any one of embodiments 34-37, wherein pulling the proximal floating element in the proximal direction while the proximal radially expandable element is collapsed and while the distal radially expandable element is expanded to engage a body cavity results in the proximal radially expandable element moving proximally within the body cavity, thereby increasing the distance between the proximal and the distal radially expandable elements.

Embodiment 39. The device of any one of embodiments 34-38, wherein pulling the proximal floating element in the proximal direction while the proximal radially expandable element is expanded to engage a body cavity and while the distal radially expandable element is collapsed results in the distal radially expandable element moving distally within the body cavity, thereby increasing the distance between the proximal and the distal radially expandable elements.

Embodiment 40. The device of any one of embodiments 34-39, wherein pulling the proximal floating element in the distal direction while the proximal radially expandable element is collapsed and while the distal radially expandable element is expanded to engage a body cavity results in the proximal radially expandable element moving distally within the body cavity, thereby decreasing the distance between the proximal and the distal radially expandable elements.

Embodiment 41. The device of any one of embodiments 34-40, wherein pulling the proximal floating element in the distal direction while the proximal radially expandable element is expanded to engage a body cavity and while the distal radially expandable element is collapsed results in the distal radially expandable element moving proximally within the body cavity, thereby decreasing the distance between the proximal and the distal radially expandable elements.

Embodiment 42. A method for locomotion of the device of any one of embodiments 34-41 through a body cavity, the method comprising: i. expanding the proximal radially expandable element radially outwardly to engage a wall of the body cavity, thereby fixing the proximal radially expandable element to a first position in the body cavity; ii. pulling the proximal floating element in the proximal direction along the elongated support while the distal radially expandable element is collapsed, thereby increasing the distance between the proximal and the distal radially expandable elements; iii. expanding the distal radially expandable element radially outwardly to engage a wall of the body cavity; iv. retracting the proximal radially expandable element radially inwardly; v. pull the proximal floating element in the distal direction, thus effecting sliding movement of the proximal radially expandable element along the elongated support and decreasing the distance between the proximal and the distal radially expandable elements; and vi. expanding the proximal radially expandable element radially outwardly to engage a wall of the body cavity, thereby fixing the proximal radially expandable element to a second position in the body cavity, optionally wherein the second position is distal to the first position.

Embodiment 43. The method of embodiment 42, further comprising: vii. pulling the proximal floating element in the proximal direction along the elongated support while the distal radially expandable element is collapsed, thereby increasing the distance between the proximal and the distal radially expandable elements.

Embodiment 44. A method for locomotion of the device of any one of embodiments 34-41 through a body cavity, the method comprising: i. expanding the distal radially expandable element radially outwardly to engage a wall of the body cavity, thereby fixing the distal radially expandable element to a first position in the body cavity; ii. pulling the proximal floating element in the distal direction along the elongated support while the proximal radially expandable element is collapsed, thereby decreasing the distance between the proximal and the distal radially expandable elements; iii. expanding the proximal radially expandable element radially outwardly to engage a wall of the body cavity; iv. retracting the distal radially expandable element radially inwardly; v. pulling the proximal floating element in the proximal direction, thus effecting sliding movement of the distal radially expandable element forward along the elongated support and increasing the distance between the proximal and the distal radially expandable elements; and vi. expanding the distal radially expandable element radially outwardly to engage a wall of the body cavity, thereby fixing the distal radially expandable element to a second position in the body cavity, optionally wherein the second position is distal to the first position.

Embodiment 45. The method of embodiment 44, further comprising: vii. pulling the proximal floating element in the distal direction along the elongated support while the proximal radially expandable element is collapsed, thereby decreasing the distance between the proximal and the distal radially expandable elements.

Embodiment 46. The device of any one of embodiments 22-29 and embodiments 34-41 further comprising an articulation element capable of effecting articulation of a distal end of the device, optionally wherein the distal end of the device is the distal end of the elongated support or the distal end of the device directly or indirectly engages the distal end of the elongated support.

Embodiment 47. The device of embodiment 46, wherein the articulation element enables camera visualization and steering of the device (e.g., one comprising an endoscope assembly), optionally wherein the device comprises machine vision elements that digitally recognize structures assisting in navigation and identifying anomalies such as lesions and polyps, optionally wherein the machine vision assists in navigation and/or transmitting location of structures such as when moving from the large to small intestine.

Embodiment 48. The device of any one of embodiments 46-47, wherein the articulation element comprises a motor.

Embodiment 49. The device of any one of embodiments 46-48, wherein the articulation element comprises one or more closed loop cables configured to effect articulation.

Embodiment 50. The method of any one of embodiments 30-33 and embodiments 42-45, further comprising capturing an image of the body cavity through a channel of the device.

Embodiment 51. The method of any one of embodiments 30-33, embodiments 42-45 and 50, further comprising delivering a substance into the body cavity through a channel of the device.

Embodiment 52. The method of any one of embodiments 30-33, embodiments 42-45 and 50-51, further comprising removing a substance into the body cavity through a channel of the device.

Embodiment 53. The method of any one of embodiments 30-33, embodiments 42-45 and 50-52, further comprising performing an operation on a tissue within the body cavity through a channel of the device.

Embodiment 54. The device or method of any one of embodiments 1-53, wherein the body cavity is a vascular body lumen, a digestive body lumen, a respiratory body lumen, or a urinary body lumen.

Embodiment 55. The device or method of claim 54, wherein the digestive body lumen is a gastrointestinal tract, optionally wherein the digestive body lumen comprises esophagus, stomach, small intestine, duodenum, jejunum, ileum, colon, and/or rectum.

Embodiment 56. The device or method of any of embodiments 1-55, wherein the expandable elements are connected to the elongated support (e.g., tubular structure such as tether) using an elastic O-ring that mechanically holds the expandable elements; using adhesive only securing the edges of the expandable elements; mechanically securing the edges of an expandable element by a deformable material such as a metal by swaging or radially compressing it around the expandable element; or by a combination thereof.

Embodiment 57. A device configured to move within a body cavity, the device comprising:
a) an elongated support;
b) a proximal radially expandable element and a distal radially expandable element positioned along the length of the elongated support, optionally wherein the radially expandable elements are independently controllably expandable,
wherein the radially expandable elements comprise outer surfaces configured to frictionally engage a wall of a body cavity, and
wherein the distal radially expandable element is fixed relative to the elongated support; and
c) a locomotion system comprising:
i) a spring connecting the proximal radially expandable element and the distal radially expandable element, such that the proximal radially expandable element is slidable along the length of the elongated support, and
ii) a cable that has one end directly or indirectly attached to the distal radially expandable element and the other end passing through the proximal radially expandable element,
wherein the locomotion system effects sliding movement of the proximal radially expandable element along the length of the elongated support, thereby effecting relative movement between the radially expandable elements and movement of the device within the body cavity.

Embodiment 58. The device of embodiment 57, wherein the spring comprises a plurality of springs connected by one or more separators.

Embodiment 59. A device configured to move within a body cavity, the device comprising:
a) an elongated support;
b) a proximal radially expandable element and a distal radially expandable element positioned along the length of the elongated support, optionally wherein the radially expandable elements are independently controllably expandable,
wherein the radially expandable elements comprise outer surfaces configured to frictionally engage a wall of a body cavity, and
wherein the distal radially expandable element is fixed relative to the elongated support; and
c) a locomotion system comprising:
i) a first cable that has one end directly or indirectly attached to the distal radially expandable element and the other end passing through the proximal radially expandable element, and
ii) a second cable that has one end directly or indirectly attached to the proximal radially expandable element,
wherein the locomotion system effects sliding movement of the proximal radially expandable element along the length of the elongated support, thereby effecting relative movement between the radially expandable elements and movement of the device within the body cavity.

Embodiment 60. The device of embodiment 59, wherein the first cable comprises a plurality of first cables and the second cable comprises a plurality of second cables, optionally the first cable and/or the second cable are configured to push and/or pull the corresponding radially expandable element.

1. A device configured to move within a body cavity, the device comprising:
a) an elongated support;
b) a proximal radially expandable element and a distal radially expandable element positioned along the length of the elongated support, optionally wherein the radially expandable elements are independently controllably expandable,
wherein the radially expandable elements comprise outer surfaces configured to frictionally engage a wall of a body cavity, and
wherein the distal radially expandable element is fixed relative to the elongated support; and
c) a locomotion system comprising:
i) a proximal locomotion element having a part that is fixed relative to the proximal radially expandable element and slidable along the length of the elongated support, such that the proximal radially expandable element is slidable along the length of the elongated support, and
ii) a distal locomotion element having a part fixed relative to the elongated support,
wherein the locomotion system effects sliding movement of the proximal radially expandable element along the length of the elongated support, thereby effecting relative movement between the radially expandable elements and movement of the device within the body cavity.

2. The device of claim 1, wherein the elongated support comprises a tubular wall and a lumen, optionally wherein the lumen is a central lumen.

3. The device of claim 2, wherein one or both of the expandable elements and/or one or both of the locomotion elements are in fluid or gas communication with one or more chambers, one or more channels, one or more tubes, and/or one or more wires in the central lumen.

4. The device of any one of claims 1-3, wherein any one or more of the expandable elements and locomotion elements are independently controlled.

5. The device of any one of claims 1-4, wherein the locomotion elements are configured to expand or collapse along the length of the elongated support, optionally wherein the locomotion elements are configured to expand or collapse only along the length of the elongated support and/or are not radially expandable.

6. The device of any one of claims 1-5, wherein the proximal and the distal radially expandable elements are capable of expanding radially outwardly to engage a wall of a body cavity, optionally wherein friction augmenting features are molded into the proximal and/or distal radially expanding elements.

7. The device of any one of claims 1-6, wherein alternating extensions and retractions of a distance between the outer surfaces of the proximal and the distal radially expandable elements effects movement of the device within the body cavity.

8. The device of any one of claims 1-7, wherein the elongated support further comprises one or more aperture on a distal end, and/or
wherein the elongated support further comprises one or more medium channels separately connected to the radially expandable elements, optionally wherein the medium comprises gas, liquid or a mixture thereof, and optionally wherein the medium comprises a vapor, and/or
wherein the elongated support further comprises one or more wires or channels for a camera or sensor, optionally wherein the sensor is a pressure sensor and/or an image sensor, and/or
wherein the elongated support houses or engages an endoscope assembly.

9. The device of any one of claims 1-8, further comprising an articulation element capable of effecting articulation of a distal end of the device, optionally wherein the distal end of the device is the distal end of the elongated support or the distal end of the device directly or indirectly engages the distal end of the elongated support.

10. The device of claim 9, wherein the articulation element enables steering of the device, optionally wherein the device comprises a machine vision element that digitally recognizes structures assisting in navigation and identifying anomalies such as lesions and polyps, optionally wherein the machine vision assists in navigation and/or transmitting location of structures such as when moving from the large intestine to the small intestine.

11. The device of any one of claims 9-10, wherein the articulation element comprises a motor.

12. The device of any one of claims 9-11, wherein the articulation element comprises one or more closed loop cables configured to effect articulation.

13. The device of any one of claims 1-12, further comprising one or more channels not in connection with the expandable elements.

14. The device of any one of claims 1-13, wherein the proximal radially expandable element is a proximal balloon.

15. The device of any one of claims 1-14, wherein the distal radially expandable element is a distal balloon.

16. The device of any one of claims 1-15, wherein the proximal radially expandable element directly or indirectly engages one or more floating elements configured to slide along the length of the elongated support, thereby sliding the proximal radially expandable element along the length of the elongated support.

17. The device of any one of claims 1-16, wherein the locomotion system comprises two longitudinally expandable elements.

18. The device of claim 17, wherein the locomotion system comprises a proximal longitudinally expandable element and a distal longitudinally expandable element, optionally wherein the longitudinally expandable elements are independently controllably expandable, and optionally wherein the longitudinally expandable elements each comprises a structure independently selected from the group consisting of a compliant balloon, a semi-compliant balloon, a pressure balloon, and a bellow.

19. The device of any one of claims 1-16, wherein the locomotion system comprising a pulley system.

20. The device of claim 19, wherein the pulley system comprises a proximal floating element, a distal wheel, and a cable connected to the proximal floating element and engaging the distal wheel.

21. The device of any one of claims 1-20, wherein the distal locomotion element comprises a part fixed to the distal expandable element.

22. A device configured to move within a body cavity, the device comprising:
a) an elongated support;
b) a proximal radially expandable element and a distal radially expandable element positioned along the length of the elongated support,
wherein the radially expandable elements comprise outer surfaces configured to frictionally engage a wall of a body cavity, and
wherein the distal radially expandable element is fixed relative to the elongated support;
c) a locomotion system comprising a proximal longitudinally expandable element and a distal longitudinally expandable element connected by a floating seal, wherein:
i) the proximal longitudinally expandable element is proximal to the proximal radially expandable element, and the floating seal is fixed relative to the proximal radially expandable element and slidable along the length of the elongated support, such that the proximal radially expandable element is slidable along the length of the elongated support, and
ii) the distal longitudinally expandable element is proximal to the distal radially expandable element, and the distal end of the distal longitudinally expandable element is fixed relative to the elongated support,
wherein the locomotion system effects sliding movement of the proximal radially expandable element along the length of the elongated support, thereby effecting relative movement between the radially expandable elements and movement of the device within the body cavity.

23. The device of claim 22, wherein the proximal and the distal longitudinally expandable elements are configured to expand or collapse along the length of the elongated support, optionally wherein the proximal and the distal longitudinally expandable elements configured to expand or collapse only along the length of the elongated support and/or are not radially expandable.

24. The device of claim 22 or 23, wherein alternating expansion and collapsing of the proximal and the distal longitudinally expandable elements do not change the distance between the proximal end of the proximal longitudinally expandable element and the distal end of the distal longitudinally expandable element.

25. The device of any one of claims 22-24, wherein the distance between the proximal end of the proximal longitudinally expandable element and the distal end of the distal longitudinally expandable element is pre-determined.

26. The device of any one of claims 22-25, wherein expansion of the proximal and/or the distal longitudinally expandable elements is effected by positive pressure, optionally wherein negative pressure is proactively and alternatively applied to the longitudinally expandable elements in order to evacuate previously applied positive pressure, and optionally wherein the proximal and/or the distal longitudinally expandable elements do not passively deflate.

27. The device of any one of claims 22-26, wherein the expansion of the proximal longitudinally expandable element and the collapsing of the distal longitudinally expandable element effects sliding movement of the proximal radially expandable element along the length of the elongated support.

28. The device of any one of claims 22-27, wherein the collapsing of the proximal longitudinally expandable element and the expansion of the distal longitudinally expandable element effects movement of the distal radially expandable element.

29. The device of any one of claims 22-28, wherein the elongated support further comprises one or more aperture on a distal end, and/or
wherein the elongated support further comprises one or more medium channels separately connected to the radially expandable elements, optionally wherein the medium comprises gas, liquid or a mixture thereof, and optionally wherein the medium comprises a vapor, and/or
wherein the elongated support further comprises one or more wires or channels for a camera or sensor, optionally wherein the sensor is a pressure sensor and/or an image sensor, and/or
wherein the elongated support houses or engages an endoscope assembly.

30. A method for locomotion of the device of any one of claims 22-29 through a body cavity, the method comprising:
i. expanding the proximal radially expandable element radially outwardly to engage a wall of the body cavity, thereby fixing the proximal radially expandable element to a first position in the body cavity;
ii. expanding (e.g., using positive pressure) the distal longitudinally expandable element along the elongated support to increase the distance between the proximal and the distal radially expandable elements;
iii. expanding the distal radially expandable element radially outwardly to engage a wall of the body cavity;
iv. retracting the proximal radially expandable element radially inwardly;
v. retracting (e.g., using negative pressure) the distal longitudinally expandable element, and/or expanding (e.g., using positive pressure) the proximal longitudinally expandable element along the elongated support, thus effecting sliding movement of the proximal radially expandable element along the elongated support and decreasing the distance between the proximal and the distal radially expandable elements; and
vi. optionally expanding the proximal radially expandable element radially outwardly to engage a wall of the body cavity, thereby fixing the proximal radially expandable element to a second position in the body cavity, optionally the second position is distal to the first position.

31. The method of claim 30, further comprising:
vii. expanding (e.g., using positive pressure) the distal longitudinally expandable element along the elongated support to increase the distance between the proximal and the distal radially expandable elements.

32. A method for locomotion of the device of any one of claims 22-29 through a body cavity, the method comprising:
i. expanding the distal radially expandable element radially outwardly to engage a wall of the body cavity, thereby fixing the distal radially expandable element to a first position in the body cavity;
ii. expanding (e.g., using positive pressure) the proximal longitudinally expandable element along the elongated support, thus effecting sliding movement of the proximal radially expandable element along the elongated support and decreasing the distance between the proximal and the distal radially expandable elements;
iii. expanding the proximal radially expandable element radially outwardly to engage a wall of the body cavity;
iv. retracting the distal radially expandable element radially inwardly;
v. retracting (e.g., using negative pressure) the proximal longitudinally expandable element, and/or expanding (e.g., using positive pressure) the distal longitudinally expandable element along the elongated support; and
vi. optionally expanding the distal radially expandable element radially outwardly to engage a wall of the body cavity, thereby fixing the distal radially expandable element to a second position in the body cavity, optionally the second position is distal to the first position.

33. The method of claim 32, further comprising:
vii. expanding (e.g., using positive pressure) the proximal longitudinally expandable element along the elongated support to decrease the distance between the proximal and the distal radially expandable elements

34. A device configured to move within a body cavity, the device comprising:
a) an elongated support;
b) a proximal radially expandable element and a distal radially expandable element positioned along the length of the elongated support,
wherein the radially expandable elements comprise outer surfaces configured to frictionally engage a wall of a body cavity, and
wherein the distal radially expandable element is fixed relative to the elongated support;
c) a pulley system comprising:
i) a proximal floating element that is fixed relative to the proximal radially expandable element and slidable along the length of the elongated support, such that the proximal radially expandable element is slidable along the length of the elongated support,
ii) a distal wheel fixed relative to the elongated support, and
iii) a cable connected to the proximal floating element and engaging the distal wheel, such that the cable is configured to pull the proximal floating element in the distal or proximal direction,
wherein the pulley system effects sliding movement of the proximal radially expandable element along the length of the elongated support, thereby effecting relative movement between the radially expandable elements and movement of the device within the body cavity.

35. The device of claim 34, wherein the cable is a closed loop cable.

36. The device of any one of claims 34-35, wherein movement of the cable effects movement of the proximal radially expandable element along the elongated support, thereby effecting alternative extensions and retractions of a distance between the outer surfaces of the proximal and the distal radially expandable elements along the length of the elongated support.

37. The device of any one of claims 34-36, wherein the radially expandable elements are independently controllably expandably, and optionally wherein the elongated support further comprises one or more medium channels separately connected to the radially expandable elements, optionally wherein the medium comprises gas, liquid or a mixture thereof, and optionally wherein the medium comprises a vapor, and/or
wherein the elongated support further comprises one or more wires or channels for a camera or sensor, optionally wherein the sensor is a pressure sensor and/or an image sensor, and/or
wherein the elongated support houses or engages an endoscope assembly.

38. The device of any one of claims 34-37, wherein pulling the proximal floating element in the proximal direction while the proximal radially expandable element is collapsed and while the distal radially expandable element is expanded to engage a body cavity results in the proximal radially expandable element moving proximally within the body cavity, thereby increasing the distance between the proximal and the distal radially expandable elements.

39. The device of any one of claims 34-38, wherein pulling the proximal floating element in the proximal direction while the proximal radially expandable element is expanded to engage a body cavity and while the distal radially expandable element is collapsed results in the distal radially expandable element moving distally within the body cavity, thereby increasing the distance between the proximal and the distal radially expandable elements.

40. The device of any one of claims 34-39, wherein pulling the proximal floating element in the distal direction while the proximal radially expandable element is collapsed and while the distal radially expandable element is expanded to engage a body cavity results in the proximal radially expandable element moving distally within the body cavity, thereby decreasing the distance between the proximal and the distal radially expandable elements.

41. The device of any one of claims 34-40, wherein pulling the proximal floating element in the distal direction while the proximal radially expandable element is expanded to engage a body cavity and while the distal radially expandable element is collapsed results in the distal radially expandable element moving proximally within the body cavity, thereby decreasing the distance between the proximal and the distal radially expandable elements.

42. A method for locomotion of the device of any one of claims 34-41 through a body cavity, the method comprising:
i. expanding the proximal radially expandable element radially outwardly to engage a wall of the body cavity, thereby fixing the proximal radially expandable element to a first position in the body cavity;
ii. pulling the proximal floating element in the proximal direction along the elongated support while the distal radially expandable element is collapsed, thereby increasing the distance between the proximal and the distal radially expandable elements;
iii. expanding the distal radially expandable element radially outwardly to engage a wall of the body cavity;
iv. retracting the proximal radially expandable element radially inwardly;
v. pull the proximal floating element in the distal direction, thus effecting sliding movement of the proximal radially expandable element along the elongated support and decreasing the distance between the proximal and the distal radially expandable elements; and
vi. expanding the proximal radially expandable element radially outwardly to engage a wall of the body cavity, thereby fixing the proximal radially expandable element to a second position in the body cavity, optionally wherein the second position is distal to the first position.

43. The method of claim 42, further comprising:
vii. pulling the proximal floating element in the proximal direction along the elongated support while the distal radially expandable element is collapsed, thereby increasing the distance between the proximal and the distal radially expandable elements.

44. A method for locomotion of the device of any one of claims 34-41 through a body cavity, the method comprising:
i. expanding the distal radially expandable element radially outwardly to engage a wall of the body cavity, thereby fixing the distal radially expandable element to a first position in the body cavity;
ii. pulling the proximal floating element in the distal direction along the elongated support while the proximal radially expandable element is collapsed, thereby decreasing the distance between the proximal and the distal radially expandable elements;
iii. expanding the proximal radially expandable element radially outwardly to engage a wall of the body cavity;
iv. retracting the distal radially expandable element radially inwardly;
v. pulling the proximal floating element in the proximal direction, thus effecting sliding movement of the distal radially expandable element forward along the elongated support and increasing the distance between the proximal and the distal radially expandable elements; and
vi. expanding the distal radially expandable element radially outwardly to engage a wall of the body cavity, thereby fixing the distal radially expandable element to a second position in the body cavity, optionally wherein the second position is distal to the first position.

45. The method of claim 44, further comprising:
vii. pulling the proximal floating element in the distal direction along the elongated support while the proximal radially expandable element is collapsed, thereby decreasing the distance between the proximal and the distal radially expandable elements.

46. The device of any one of claims 22-29 and claims 34-41 further comprising an articulation element capable of effecting articulation of a distal end of the device, optionally wherein the distal end of the device is the distal end of the elongated support or the distal end of the device directly or indirectly engages the distal end of the elongated support.

47. The device of claim 46, wherein the articulation element enables camera visualization and steering of the device (e.g., one comprising an endoscope assembly), optionally wherein the device comprises machine vision elements that digitally recognize structures assisting in navigation and identifying anomalies such as lesions and polyps, optionally wherein the machine vision assists in navigation and/or transmitting location of structures such as when moving from the large to small intestine.

48. The device of any one of claims 46-47, wherein the articulation element comprises a motor.

49. The device of any one of claims 46-48, wherein the articulation element comprises one or more closed loop cables configured to effect articulation.

50. The method of any one of claims 30-33 and claims 42-45, further comprising capturing an image of the body cavity through a channel of the device.

51. The method of any one of claims 30-33, claims 42-45 and 50, further comprising delivering a substance into the body cavity through a channel of the device.

52. The method of any one of claims 30-33, claims 42-45 and 50-51, further comprising removing a substance into the body cavity through a channel of the device.

53. The method of any one of claims 30-33, claims 42-45 and 50-52, further comprising performing an operation on a tissue within the body cavity through a channel of the device.

54. The device or method of any one of claims 1-53, wherein the body cavity is a vascular body lumen, a digestive body lumen, a respiratory body lumen, or a urinary body lumen.

55. The device or method of claim 54, wherein the digestive body lumen is a gastrointestinal tract, optionally wherein the digestive body lumen comprises esophagus, stomach, small intestine, duodenum, jejunum, ileum, colon, and/or rectum.

56. The device or method of any of claims 1-55, wherein the expandable elements are connected to the elongated support (e.g., tubular structure such as tether) using an elastic O-ring that mechanically holds the expandable elements; using adhesive only securing the edges of the expandable elements; mechanically securing the edges of an expandable element by a deformable material such as a metal by swaging or radially compressing it around the expandable element; or by a combination thereof.

57. A device configured to move within a body cavity, the device comprising:
a) an elongated support;
b) a proximal radially expandable element and a distal radially expandable element positioned along the length of the elongated support, optionally wherein the radially expandable elements are independently controllably expandable,
wherein the radially expandable elements comprise outer surfaces configured to frictionally engage a wall of a body cavity, and
wherein the distal radially expandable element is fixed relative to the elongated support; and
c) a locomotion system comprising:
i) a spring connecting the proximal radially expandable element and the distal radially expandable element, such that the proximal radially expandable element is slidable along the length of the elongated support, and
ii) a cable that has one end directly or indirectly attached to the distal radially expandable element and the other end passing through the proximal radially expandable element,
wherein the locomotion system effects sliding movement of the proximal radially expandable element along the length of the elongated support, thereby effecting relative movement between the radially expandable elements and movement of the device within the body cavity.

58. The device of claim 57, wherein the spring comprises a plurality of springs connected by one or more separators.

59. A device configured to move within a body cavity, the device comprising:
a) an elongated support;
b) a proximal radially expandable element and a distal radially expandable element positioned along the length of the elongated support, optionally wherein the radially expandable elements are independently controllably expandable,
wherein the radially expandable elements comprise outer surfaces configured to frictionally engage a wall of a body cavity, and
wherein the distal radially expandable element is fixed relative to the elongated support; and
c) a locomotion system comprising:
i) a first cable that has one end directly or indirectly attached to the distal radially expandable element and the other end passing through the proximal radially expandable element, and
ii) a second cable that has one end directly or indirectly attached to the proximal radially expandable element,
wherein the locomotion system effects sliding movement of the proximal radially expandable element along the length of the elongated support, thereby effecting relative movement between the radially expandable elements and movement of the device within the body cavity.

60. The device of claim 59, wherein the first cable comprises a plurality of first cables and the second cable comprises a plurality of second cables, optionally the first cable and/or the second cable are configured to push and/or pull the corresponding radially expandable element.
